SEQUENCE LISTING (107 sequences)

SEQ ID NO: 1 - protein, 8 aa, Artificial Sequence
  MGU10 CDRH1
GFAFSNYG

SEQ ID NO: 2 - protein, 8 aa, Artificial Sequence
  MGU10 CDRH2
IWHDGSLK

SEQ ID NO: 3 - protein, 14 aa, Artificial Sequence
  MGU10 CDRH3
TVWYLETPDDGFDI

SEQ ID NO: 4 - protein, 7 aa, Artificial Sequence
  MGU10 CDRL1
HGHTSKA

SEQ ID NO: 5 - protein, 7 aa, Artificial Sequence
  MGU10 CDRL2
VNSDGSH

SEQ ID NO: 6 - protein, 9 aa, Artificial Sequence
  MGU10 CDRL3
QAWDSGIWV

SEQ ID NO: 7 - protein, 121 aa, Artificial Sequence
  MGU10 VH
QVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDDGFDIWGRGTMVTVSS

SEQ ID NO: 8 - protein, 111 aa, Artificial Sequence
  MGU10 VL
QLVLTQPPSASASLGVSVTLTCTLSHGHTSKAIAWHQQQPGKGPRYLMKVNSDGSHTKGAAVPDRFSGSTSGAERHFTISNLQSDDEADYYCQAWDSGIWVFGGGTKLTVL

SEQ ID NO: 9 - protein, 451 aa, Artificial Sequence
  MGU10 heavy chain
QVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDDGFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 10 - protein, 217 aa, Artificial Sequence
  MGU10 light chain
QLVLTQPPSASASLGVSVTLTCTLSHGHTSKAIAWHQQQPGKGPRYLMKVNSDGSHTKGAAVPDRFSGSTSGAERHFTISNLQSDDEADYYCQAWDSGIWVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 11 - protein, 121 aa, Artificial Sequence
  MGU10v1 VH
QVQLVESGGGVVQPGRSLRLSCAASGFAFSNYGMHWVRQAPGKGLEWVAVIWHDGSLKYYTQSVKGRFTISRDNAKNTLFLQMDSLSADDTAMYYCTVWYLETPDDGFDIWGQGTMVTVSS

SEQ ID NO: 12 - protein, 14 aa, Artificial Sequence
  MGU10v2 CDRH3
TVWYLETPDEGFDI

SEQ ID NO: 13 - protein, 121 aa, Artificial Sequence
  MGU10v2 VH
QVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDEGFDIWGRGTMVTVSS

SEQ ID NO: 14 - protein, 9 aa, Artificial Sequence
  MGU10v3 CDRL3
QAWESGIWV

SEQ ID NO: 15 - protein, 111 aa, Artificial Sequence
  MGU10v3 VL
QLVLTQPPSASASLGVSVTLTCTLSHGHTSKAIAWHQQQPGKGPRYLMKVNSDGSHTKGAAVPDRFSGSTSGAERHFTISNLQSDDEADYYCQAWESGIWVFGGGTKLTVL

SEQ ID NO: 16 - protein, 121 aa, Artificial Sequence
  MGU10v4 VH
QVQLVESGGGVVQPGRSLRLSCAASGFAFSNYGMHWVRQAPGKGLEWVAVIWHDGSLKYYTQSVKGRFTISRDNAKNTLFLQMDSLSADDTAMYYCTVWYLETPDEGFDIWGQGTMVTVSS

SEQ ID NO: 17 - protein, 8 aa, Artificial Sequence
  MGH2 CDRH1
GFSFSSYA

SEQ ID NO: 18 - protein, 8 aa, Artificial Sequence
  MGH2 CDRH2
TRYDGSNK

SEQ ID NO: 19 - protein, 14 aa, Artificial Sequence
  MGH2 CDRH3
AKVGDGTVAGTIDY

SEQ ID NO: 20 - protein, 11 aa, Artificial Sequence
  MGH2 CDRL1
QSLVYSDGNTY

SEQ ID NO: 21 - skipped

SEQ ID NO: 22 - protein, 9 aa, Artificial Sequence
  MGH2 CDRL2 long
LIYKVSNRD

SEQ ID NO: 23 - protein, 8 aa, Artificial Sequence
  MGH2 CDRL3
MQGTHWWT

SEQ ID NO: 24 - protein, 121 aa, Artificial Sequence
  MGH2 VH
QVQLVESGGGVVQPGGSLRLSCTASGFSFSSYAMHWVRQAPGKGLEWVAYTRYDGSNKFYLDSVQGRFTISRDNSKNTLYLEMDSLRLEDTAVYFCAKVGDGTVAGTIDYWGQGTLVTVSS

SEQ ID NO: 25 - protein, 111 aa, Artificial Sequence
  MGH2 VL
YIVMTQSPLSLPVTLGQPASISCRSSQSLVYSDGNTYLNWYQQRPGQSPRRLIYKVSNRDSGVPDRFSGSGSGTDFTLKISRVEAEDVGVYYCMQGTHWWTFGQGTKVEIK

SEQ ID NO: 26 - protein, 451 aa, Artificial Sequence
  MGH2 heavy chain
QVQLVESGGGVVQPGGSLRLSCTASGFSFSSYAMHWVRQAPGKGLEWVAYTRYDGSNKFYLDSVQGRFTISRDNSKNTLYLEMDSLRLEDTAVYFCAKVGDGTVAGTIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 27 - protein, 218 aa, Artificial Sequence
  MGH2 light chain
YIVMTQSPLSLPVTLGQPASISCRSSQSLVYSDGNTYLNWYQQRPGQSPRRLIYKVSNRDSGVPDRFSGSGSGTDFTLKISRVEAEDVGVYYCMQGTHWWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 28 - protein, 8 aa, Artificial Sequence
  MGH2v1 CDRL3
MQGTHFWT

SEQ ID NO: 29 - protein, 111 aa, Artificial Sequence
  MGH2v1 VL
YIVMTQSPLSLPVTLGQPASISCRSSQSLVYSDGNTYLNWYQQRPGQSPRRLIYKVSNRDSGVPDRFSGSGSGTDFTLKISRVEAEDVGVYYCMQGTHFWTFGQGTKVEIK

SEQ ID NO: 30 - protein, 330 aa, Artificial Sequence
  heavy chain constant regions
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 31 - protein, 106 aa, Artificial Sequence
  lambda light chain constant regions
GQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 32 - protein, 107 aa, Artificial Sequence [sequence omitted]

SEQ ID NO: 33 - protein, 397 aa, Plasmodium falciparum
MMRKLAILSVSSFLFVEALFQEYQCYGSSSNTRVLNELNYDNAGTNLYNELEMNYYGKQENWYSLKKNSRSLGENDDGNNEDNEKLRKPKHKKLKQPADGNPDPNANPNVDPNANPNVDPNANPNVDPNANPNANPNANPNANPNANPNANPNANPNANPNANPNANPNANPNANPNANPNANPNANPNANPNANPNVDPNANPNANPNANPNANPNANPNANPNANPNANPNANPNANPNANPNANPNANPNANPNANPNANPNANPNANPNKNNQGNGQGHNMPNDPNRNVDENANANSAVKNNNNEEPSDKHIKEYLNKIQNSLSTEWSPCSVTCGNGIQVRIKPGSANKPKDELDYANDIEKKICKMEKCSSVFNVVNSSIGLIMVLSFLFLN

SEQ ID NO: 34 - protein, 18 aa, Artificial Sequence
  NANP-peptide
NANPNANPNANPNANPNA

SEQ ID NO: 35 - protein, 15 aa, Artificial Sequence
  NPDP-peptide
KQPADGNPDPNANPN

SEQ ID NO: 36 - DNA, 363 bp, Artificial Sequence
  MGU10v1 VH
caggtgcagctggtggagagcggaggaggagtggtgcagccaggccggtccctgagactgtcttgcgccgccagcggcttcgccttttccaactacggaatgcactgggtgcggcaggcacctggcaagggactggagtgggtggccgtgatctggcacgacggatccctgaagtactatacacagtctgtgaagggcaggttcaccatctctcgcgacaacgccaagaatacactgtttctgcagatggattctctgagcgccgacgataccgccatgtactattgtacagtgtggtatctggagacccccgacgatggcttcgatatctggggccagggcaccatggtgacagtgagctcc

SEQ ID NO: 37 - DNA, 363 bp, Artificial Sequence
  MGU10v2 VH
caggtgcagctggtggagagcggaggaggagtggtgcagccaggccggtccctgagactgtcttgcgccgccagcggcttcgccttttccaactacggcatgaattgggtgcggcaggcacctggcaagggactggagtgggtggccgtgatctggcacgacggatccctgaagtactatacacagtctgtgaagggcaggttcaccatctctcgcgacaacgccaagaatacactgtttctgcagatggattctctgagcgccgacgataccgccatgtactattgtacagtgtggtatctggagacccccgacgagggcttcgatatctggggcaggggcaccatggtgacagtgagctcc

SEQ ID NO: 38 - DNA, 333 bp, Artificial Sequence
  MGU10v3 VL
cagctggtgctgacacagccacctagcgcctccgcctctctgggcgtgagcgtgaccctgacatgcaccctgtcccacggccacacctctaaggcaatcgcatggcaccagcagcagccaggcaagggaccacggtacctgatgaaggtgaacagcgacggatcccacacaaagggagcagcagtgccagatcggttcagcggctccacatctggcgccgagagacactttaccatctctaatctgcagagcgacgatgaggccgactactattgtcaggcctgggagtccggaatctgggtgttcggaggaggaacaaagctgaccgtgctg

SEQ ID NO: 39 - DNA, 333 bp, Artificial Sequence
  MGH2v1 VL
tacatcgtgatgacccagtcccccctgtctctgcctgtgacactgggccagcctgcctctatcagctgccggagctcccagagcctggtgtactccgacggcaacacctacctgaattggtatcagcagaggccaggacagtccccacggagactgatctataaggtgtctaacagggacagcggagtgccagatcgcttctccggatctggaagcggaaccgactttacactgaagatctctcgggtggaggccgaggatgtgggcgtgtactattgtatgcagggcacccacttctggacatttggccagggcacaaaggtggagatcaag

SEQ ID NO: 40 - DNA, 363 bp, Artificial Sequence
  MGU10 VHv1
caggtgcagctggtggagagcggcggcggcgtggtgcagcccggcagaagcctgagactgagctgcgccgccagcggcttcgccttcagcaactacggcatgaactgggtgagacaggcccccggcaagggcctggagtgggtggccgtgatctggcacgacggcagcctgaagtactacacccagagcgtgaagggcagattcaccatcagcagagacaacgccaagaacaccctgttcctgcagatggacagcctgagcgccgacgacaccgccatgtactactgcaccgtgtggtacctggagacccccgacgacggcttcgacatctggggcagaggcaccatggtgaccgtgagcagc

SEQ ID NO: 41 - DNA, 363 bp, Artificial Sequence
  MGU10 VHv2
caggttcagttagtcgagagtgggggcggggttgtgcaaccagggagatcgttgaggctcagctgcgcagccagtggcttcgccttcagtaattacggcatgaactgggttaggcaggctcctggcaaaggtttggagtgggtagcagtgatttggcatgacggctctttgaaatattacacacagagtgtgaaaggaagattcacaatcagcagagacaacgccaagaatactctgttcctgcagatggattccctgtcagccgacgacacggccatgtattactgtaccgtgtggtatctcgagacacccgatgatggcttcgacatctgggggagaggcaccatggttaccgtgagcagc

SEQ ID NO: 42 - DNA, 363 bp, Artificial Sequence
  MGU10 VHv3
caggtccagctggtcgagtcaggcgggggcgttgtccaaccgggacgctctttgcgattatcttgcgcagcgtccggctttgcgttcagtaattatggcatgaactgggtccgacaagctcccggaaaagggctggaatgggttgcggtgatttggcatgacggaagcttgaagtactatacgcagtcagtgaaaggaaggttcacaatttcacgggataatgcgaagaacactctattcctacagatggactcactttccgctgacgacaccgccatgtattactgcaccgtttggtacttggaaacgccggacgacgggtttgatatctggggcagagggacaatggttaccgtttcctca

SEQ ID NO: 43 - DNA, 1353 bp, Artificial Sequence
  MGU10 heavy chain variant 1
caggtgcagctggtggagagcggcggcggcgtggtgcagcccggcagaagcctgagactgagctgcgccgccagcggcttcgccttcagcaactacggcatgaactgggtgagacaggcccccggcaagggcctggagtgggtggccgtgatctggcacgacggcagcctgaagtactacacccagagcgtgaagggcagattcaccatcagcagagacaacgccaagaacaccctgttcctgcagatggacagcctgagcgccgacgacaccgccatgtactactgcaccgtgtggtacctggagacccccgacgacggcttcgacatctggggcagaggcaccatggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgctgcacgaggccctgcacagccactacacccagaagagcctgagcctgagccccggcaag

SEQ ID NO: 44 - DNA, 1353 bp, Artificial Sequence
  MGU10 heavy chain variant 2
caggttcagttagtcgagagtgggggcggggttgtgcaaccagggagatcgttgaggctcagctgcgcagccagtggcttcgccttcagtaattacggcatgaactgggttaggcaggctcctggcaaaggtttggagtgggtagcagtgatttggcatgacggctctttgaaatattacacacagagtgtgaaaggaagattcacaatcagcagagacaacgccaagaatactctgttcctgcagatggattccctgtcagccgacgacacggccatgtattactgtaccgtgtggtatctcgagacacccgatgatggcttcgacatctgggggagaggcaccatggttaccgtgagcagcgcttccaccaaaggcccatccgtcttcccactggccccctcctcaaaaagtacctcaggagggaccgccgccttgggctgccttgtaaaagactacttccctgagcccgttactgtctcttggaactccggcgctctgacctccggcgttcatacgtttcctgccgtgcttcagtccagtggcttgtattccctgagctctgtagtcaccgtgccgagcagtagcctcggcacacagacgtacatatgtaacgtgaatcacaagccatctaacactaaagtcgataaaaaagtagagcctaaaagctgtgataaaacccacacatgtccgccatgtcccgcgcccgaactgttgggcgggcccagtgtgttcctattcccaccaaaaccgaaagacaccttgatgatcagtcgcacacctgaggtaacctgcgtggtcgtcgacgtctcccacgaagaccccgaggtcaagtttaactggtatgttgatggcgtcgaggtacacaatgcaaaaaccaaaccaagggaagaacaatataatagtacatatcgcgtggtgagtgtcctcaccgtgctccaccaggactggctaaatgggaaggagtataaatgcaaggtgagcaacaaggcactcccggccccaattgagaaaaccatttccaaagccaagggccaaccacgagaaccacaggtctacaccctcccaccttcacgcgacgagttgacaaagaatcaagtgtctctcacctgtcttgtgaaggggttttatcccagtgatatcgcggtggaatgggagagcaatggacaaccagagaacaactataagaccaccccgcctgtcctggattccgacggatctttcttcctttattcaaagttgaccgtggacaagtcccggtggcagcaagggaatgtgttcagctgctcggtgctccatgaggcccttcacagccactatacgcagaagagcttatctctgagccctgggaag

SEQ ID NO: 45 - DNA, 1353 bp, Artificial Sequence
  MGU10 heavy chain variant 3
caggtccagctggtcgagtcaggcgggggcgttgtccaaccgggacgctctttgcgattatcttgcgcagcgtccggctttgcgttcagtaattatggcatgaactgggtccgacaagctcccggaaaagggctggaatgggttgcggtgatttggcatgacggaagcttgaagtactatacgcagtcagtgaaaggaaggttcacaatttcacgggataatgcgaagaacactctattcctacagatggactcactttccgctgacgacaccgccatgtattactgcaccgtttggtacttggaaacgccggacgacgggtttgatatctggggcagagggacaatggttaccgtttcctcagccagtacgaaggggccctcagtatttccgctagcgccgagctcaaagtcgacatctgggggcacagcagcactgggatgtctggtcaaagattacttccccgagcctgtaaccgttagttggaatagtggtgccttaacgagtggggttcatacatttccagcggtactccagtcctcagggctctactccttatcaagcgtcgttacagtcccaagttcatcgctaggtactcaaacttacatctgcaatgttaaccataagcccagcaataccaaagtcgacaaaaaagtcgaaccgaagtcctgcgacaagacgcacacgtgtccaccttgtcctgccccggagttattgggcggcccgtcggtgttcttgtttcctcccaaaccgaaggataccctaatgatttcgaggacgccagaagtaacatgtgttgtggtcgatgtatctcatgaagacccagaggttaagttcaactggtatgtcgatggcgtcgaagtacacaacgcaaagaccaaacccagggaagaacagtacaatagtacttatagggttgtatcagtacttacggtcctgcatcaggactggcttaacggtaaagagtacaaatgtaaggtgtctaataaggcactgcccgcgccaattgaaaaaaccatctcgaaagctaagggccagcccagagaacctcaagtgtacacgcttccgccgagtcgcgacgaactgactaagaaccaggtttctctgacttgcctagttaaaggtttctacccgtcggacatagcagtcgaatgggaaagcaacggccagccggagaacaactacaagaccacgcctccggtgctcgattcggatgggtctttctttttatattcgaaattaaccgtggataaaagtcggtggcaacaaggtaatgttttcagttgttctgtccttcacgaagccctacattcgcactacacgcaaaagagtttaagtttgtcaccggggaag

SEQ ID NO: 46 - DNA, 333 bp, Artificial Sequence
  MGU10 VLv1
cagctggtgctgacccagccccccagcgccagcgccagcctgggcgtgagcgtgaccctgacctgcaccctgagccacggccacaccagcaaggccatcgcctggcaccagcagcagcccggcaagggccccagatacctgatgaaggtgaacagcgacggcagccacaccaagggcgccgccgtgcccgacagattcagcggcagcaccagcggcgccgagagacacttcaccatcagcaacctgcagagcgacgacgaggccgactactactgccaggcctgggacagcggcatctgggtgttcggcggcggcaccaagctgaccgtgctg

SEQ ID NO: 47 - DNA, 333 bp, Artificial Sequence
  MGU10 VLv2
cagttggtgttgacccaaccaccgtctgcctctgcaagtctgggggtgtctgtgacacttacttgcacgctgtctcacgggcacacaagcaaggccattgcttggcaccaacagcagcctggcaagggacctcggtacttgatgaaggtcaactccgacggcagtcataccaaaggtgcagcagtgccggatagattttccggctccacaagtggcgcggagcgccactttaccatctccaaccttcagagcgatgacgaagctgattattattgtcaggcctgggactcaggcatatgggtattcgggggggggaccaagctcaccgtgtta

SEQ ID NO: 48 - DNA, 333 bp, Artificial Sequence
  MGU10 VLv3
caactcgttctcacccaaccgccttcagccagcgccagtctcggagtatcggtgacccttacgtgcacactctcacacggccacacgtcgaaggctatagcctggcatcagcaacaaccgggcaaaggaccgcgttatctaatgaaggtcaattccgacggatctcatacaaaaggcgccgccgtacctgaccgctttagcgggagtacgtccggggcagagcgtcatttcacgataagtaatctccagtcggatgacgaggctgactattattgtcaggcctgggactcaggtatttgggtatttggaggggggaccaagttgacggtctta

SEQ ID NO: 49 - DNA, 651 bp, Artificial Sequence
  MGU10 light chain variant 1
cagctggtgctgacccagccccccagcgccagcgccagcctgggcgtgagcgtgaccctgacctgcaccctgagccacggccacaccagcaaggccatcgcctggcaccagcagcagcccggcaagggccccagatacctgatgaaggtgaacagcgacggcagccacaccaagggcgccgccgtgcccgacagattcagcggcagcaccagcggcgccgagagacacttcaccatcagcaacctgcagagcgacgacgaggccgactactactgccaggcctgggacagcggcatctgggtgttcggcggcggcaccaagctgaccgtgctgggccagcccaaggccgcccccagcgtgaccctgttcccccccagcagcgaggagctgcaggccaacaaggccaccctggtgtgcctgatcagcgacttctaccccggcgccgtgaccgtggcctggaaggccgacagcagccccgtgaaggccggcgtggagaccaccacccccagcaagcagagcaacaacaagtacgccgccagcagctacctgagcctgacccccgagcagtggaagagccacagaagctacagctgccaggtgacccacgagggcagcaccgtggagaagaccgtggcccccaccgagtgcagc

SEQ ID NO: 50 - DNA, 651 bp, Artificial Sequence
  MGU10 light chain variant 2
cagttggtgttgacccaaccaccgtctgcctctgcaagtctgggggtgtctgtgacacttacttgcacgctgtctcacgggcacacaagcaaggccattgcttggcaccaacagcagcctggcaagggacctcggtacttgatgaaggtcaactccgacggcagtcataccaaaggtgcagcagtgccggatagattttccggctccacaagtggcgcggagcgccactttaccatctccaaccttcagagcgatgacgaagctgattattattgtcaggcctgggactcaggcatatgggtattcgggggggggaccaagctcaccgtgttagggcaaccgaaagcggcacccagtgtgaccctgtttccccccagcagtgaggaactccaggcaaataaggccactttggtctgtctgattagtgatttttatcccggggcagtcaccgtggcttggaaagcggactcttctcccgtaaaagccggagtcgagaccactacaccgtctaagcagagtaataacaaatatgctgctagctcttacctgtccctgacaccagaacagtggaagtcccataggagttatagctgccaggtcacacacgaggggagcaccgtggagaagacagttgcacccactgagtgctcc

SEQ ID NO: 51 - DNA, 651 bp, Artificial Sequence
  MGU10 light chain variant 3
caactcgttctcacccaaccgccttcagccagcgccagtctcggagtatcggtgacccttacgtgcacactctcacacggccacacgtcgaaggctatagcctggcatcagcaacaaccgggcaaaggaccgcgttatctaatgaaggtcaattccgacggatctcatacaaaaggcgccgccgtacctgaccgctttagcgggagtacgtccggggcagagcgtcatttcacgataagtaatctccagtcggatgacgaggctgactattattgtcaggcctgggactcaggtatttgggtatttggaggggggaccaagttgacggtcttaggccagcctaaggcggcgccgtctgttactttattccctccttcttcggaggagcttcaggccaacaaggccacccttgtatgtcttatatccgacttttatcctggagccgttactgttgcgtggaaggccgactcgtcgcctgtcaaggccggggtcgagactacgaccccttcaaagcaaagtaacaataagtacgctgcaagctcttatctgtcactaacgcctgagcagtggaagtcgcacagatcatatagctgccaggttacccatgaagggagcactgtggaaaaaaccgttgcaccaactgaatgcagc

SEQ ID NO: 52 - DNA, 363 bp, Artificial Sequence
  MGH2 VHv1
caggtgcagctggtggagagcggcggcggcgtggtgcagcccggcggcagcctgagactgagctgcaccgccagcggcttcagcttcagcagctacgccatgcactgggtgagacaggcccccggcaagggcctggagtgggtggcctacaccagatacgacggcagcaacaagttctacctggacagcgtgcagggcagattcaccatcagcagagacaacagcaagaacaccctgtacctggagatggacagcctgagactggaggacaccgccgtgtacttctgcgccaaggtgggcgacggcaccgtggccggcaccatcgactactggggccagggcaccctggtgaccgtgagcagc

SEQ ID NO: 53 - DNA, 363 bp, Artificial Sequence
  MGH2 VHv2
caggtgcagctggtggaaagcggggggggagtcgtgcagcctggcggctctttgcggctgtcttgtacggcttctggattctcattctcttcctacgccatgcactgggtccgccaggcgcccgggaaggggctggaatgggttgcctacacaaggtatgatggttcaaacaagttctacttagattcagtgcagggtagattcactataagccgggacaatagtaagaacactctctacctagaaatggactctctcagactggaagataccgctgtgtacttctgtgctaaggttggggacggcaccgttgccggcacaatcgactattggggacaagggaccctcgtcacagtcagctcg

SEQ ID NO: 54 - DNA, 363 bp, Artificial Sequence
  MGH2 VHv3
caagtccagcttgtcgagtcggggggaggggttgtccagcctggtggtagcttacgcctgagttgtacagcatcggggtttagcttctcttcctatgcgatgcactgggtgagacaggctcccggaaagggcttagagtgggtggcctacactcggtatgacggttcgaataagttttacttagacagcgttcagggtaggttcaccatctcacgtgataatagtaagaatacattatatcttgagatggacagccttcggttggaggatactgccgtctacttttgtgctaaggtaggcgatggtacggtagcaggcacgatagattactggggccaaggaacgttggtcactgtctcttca

SEQ ID NO: 55 - DNA, 1353 bp, Artificial Sequence
  MGH2 heavy chain variant 1
caggtgcagctggtggagagcggcggcggcgtggtgcagcccggcggcagcctgagactgagctgcaccgccagcggcttcagcttcagcagctacgccatgcactgggtgagacaggcccccggcaagggcctggagtgggtggcctacaccagatacgacggcagcaacaagttctacctggacagcgtgcagggcagattcaccatcagcagagacaacagcaagaacaccctgtacctggagatggacagcctgagactggaggacaccgccgtgtacttctgcgccaaggtgggcgacggcaccgtggccggcaccatcgactactggggccagggcaccctggtgaccgtgagcagcgccagcaccaagggccccagcgtgttccccctggcccccagcagcaagagcaccagcggcggcaccgccgccctgggctgcctggtgaaggactacttccccgagcccgtgaccgtgagctggaacagcggcgccctgaccagcggcgtgcacaccttccccgccgtgctgcagagcagcggcctgtacagcctgagcagcgtggtgaccgtgcccagcagcagcctgggcacccagacctacatctgcaacgtgaaccacaagcccagcaacaccaaggtggacaagaaggtggagcccaagagctgcgacaagacccacacctgccccccctgccccgcccccgagctgctgggcggccccagcgtgttcctgttcccccccaagcccaaggacaccctgatgatcagcagaacccccgaggtgacctgcgtggtggtggacgtgagccacgaggaccccgaggtgaagttcaactggtacgtggacggcgtggaggtgcacaacgccaagaccaagcccagagaggagcagtacaacagcacctacagagtggtgagcgtgctgaccgtgctgcaccaggactggctgaacggcaaggagtacaagtgcaaggtgagcaacaaggccctgcccgcccccatcgagaagaccatcagcaaggccaagggccagcccagagagccccaggtgtacaccctgccccccagcagagacgagctgaccaagaaccaggtgagcctgacctgcctggtgaagggcttctaccccagcgacatcgccgtggagtgggagagcaacggccagcccgagaacaactacaagaccaccccccccgtgctggacagcgacggcagcttcttcctgtacagcaagctgaccgtggacaagagcagatggcagcagggcaacgtgttcagctgcagcgtgctgcacgaggccctgcacagccactacacccagaagagcctgagcctgagccccggcaag

SEQ ID NO: 56 - DNA, 1353 bp, Artificial Sequence
  MGH2 heavy chain variant 2
caggtgcagctggtggaaagcggggggggagtcgtgcagcctggcggctctttgcggctgtcttgtacggcttctggattctcattctcttcctacgccatgcactgggtccgccaggcgcccgggaaggggctggaatgggttgcctacacaaggtatgatggttcaaacaagttctacttagattcagtgcagggtagattcactataagccgggacaatagtaagaacactctctacctagaaatggactctctcagactggaagataccgctgtgtacttctgtgctaaggttggggacggcaccgttgccggcacaatcgactattggggacaagggaccctcgtcacagtcagctcggccagtaccaagggccccagtgtgttccctttggccccttctagcaaatcaacgtcaggggggacagccgcccttgggtgtcttgtgaaagattattttcctgagccggtgaccgtttcctggaatagtggagcactgacaagcggcgtacataccttcccagcagtgctccaatcaagtgggctatacagtctgagtagcgtggtcaccgtgccatcttcctctctgggaactcaaacctatatttgcaacgttaatcacaaaccttctaatacgaaagtcgataagaaggtagaaccgaagtcctgcgacaaaacccacacatgccctccgtgcccagcccctgagctactgggcggcccctctgtgtttttgtttcccccaaaacctaaggataccttaatgatctcaagaacacccgaggtgacctgcgtcgtggtagatgtttctcacgaggaccctgaggttaaatttaattggtacgtggacggcgtggaggtccataacgccaagactaaaccaagagaagagcagtacaactccacatacagggttgtgtctgtgctgacggtcttacaccaagactggcttaacggcaaagagtataaatgcaaggtgagtaataaagcgcttcctgccccaatcgaaaagaccatcagtaaagccaaaggacagcccagggagcctcaagtctacacgttacctccctcaagggatgagctcaccaaaaaccaggtgtctctgacctgcctggttaagggcttttatccttcagacatcgctgttgagtgggaatcaaatgggcagccagaaaataactataaaactacccctcctgtgctggacagtgacggttcgttcttcctctatagtaagttgaccgtggataaaagccgatggcagcagggaaatgtgttcagctgctctgtactacatgaggccctccacagtcactatacgcagaagtcactgagtctaagtccagggaag

SEQ ID NO: 57 - DNA, 1353 bp, Artificial Sequence
  MGH2 heavy chain variant 3
caagtccagcttgtcgagtcggggggaggggttgtccagcctggtggtagcttacgcctgagttgtacagcatcggggtttagcttctcttcctatgcgatgcactgggtgagacaggctcccggaaagggcttagagtgggtggcctacactcggtatgacggttcgaataagttttacttagacagcgttcagggtaggttcaccatctcacgtgataatagtaagaatacattatatcttgagatggacagccttcggttggaggatactgccgtctacttttgtgctaaggtaggcgatggtacggtagcaggcacgatagattactggggccaaggaacgttggtcactgtctcttcagcatctaccaagggtccatctgtcttcccactggccccatcctccaaaagcacgagcggaggcaccgctgctctagggtgtctcgtcaaggactactttcccgagccagtgacagttagttggaattccggtgcacttacgtcgggggttcacacattcccagcagtgctgcagtcgagcggcctctacagcttgtcctcagtcgtaactgttccatccagttcgctcgggactcagacttacatctgcaatgtaaaccacaaaccatccaatacgaaggtggataaaaaggttgagcctaagtcatgcgacaagacacatacgtgcccaccatgtcccgcgccagagttgcttggcggacccagcgtctttctgttcccacctaaacccaaggacacgttgatgattagcaggacccccgaagttacttgtgtcgtggtggatgtaagccatgaagacccagaggtgaaatttaactggtatgtggatggagttgaagtccataatgcgaagacaaaacctcgagaggaacaatataactccacgtatcgagtcgtgtccgtactcacagtgttacatcaagattggttaaatggtaaagagtacaaatgcaaggtttcgaataaagcactgccagcgccgatcgaaaagactatctcaaaggcaaaaggccagccccgggagcctcaagtatatacgctgccgccatcgcgcgacgagttaacaaaaaaccaagtatcgttgacgtgtttggtgaaaggtttttacccttcggatatagccgtggagtgggaatccaacggtcaaccagagaacaactataagacaaccccaccagtcttagatagtgatggctctttcttcctctatagtaaactaacggtcgataaatctcgctggcagcagggcaacgtcttttcgtgttcggttttacatgaagctctacatagtcactatacccagaagagtctatctctaagccccggcaag

SEQ ID NO: 58 - DNA, 333 bp, Artificial Sequence
  MGH2 VLv1
tacatcgtgatgacccagagccccctgagcctgcccgtgaccctgggccagcccgccagcatcagctgcagaagcagccagagcctggtgtacagcgacggcaacacctacctgaactggtaccagcagagacccggccagagccccagaagactgatctacaaggtgagcaacagagacagcggcgtgcccgacagattcagcggcagcggcagcggcaccgacttcaccctgaagatcagcagagtggaggccgaggacgtgggcgtgtactactgcatgcagggcacccactggtggaccttcggccagggcaccaaggtggagatcaag

SEQ ID NO: 59 - DNA, 333 bp, Artificial Sequence
  MGH2 VLv2
tacattgttatgacccagagtcctctttcactgcctgtgacccttggccagcctgcctccatcagctgccggtccagccaatctctcgtgtactccgacggcaatacctacctgaactggtatcaacaacgacccggccagtcacccagacgcctgatctataaggtcagcaatcgggacagcggcgtcccggataggttctcaggttcaggttcaggcaccgatttcacgctgaaaattagtagagttgaggcagaagatgtcggcgtgtactactgtatgcagggtacccattggtggacctttgggcagggcacaaaagtagagattaag

SEQ ID NO: 60 - DNA, 333 bp, Artificial Sequence
  MGH2 VLv3
tacatagtaatgacgcagagtcctctgtccttaccagttacactgggccaacctgcatctatatcgtgtcgatcatctcagtccctcgtgtactcagatggaaatacgtatttgaactggtatcaacagcgtccgggacagagccctcgccgtttaatctacaaagttagtaaccgagacagtggcgttcctgaccgtttctcaggatcaggttccgggacagatttcaccttaaaaataagcagggttgaagctgaggacgtgggggtttattattgcatgcagggtacccactggtggacttttggacagggtacgaaggttgagatcaag

SEQ ID NO: 61 - DNA, 654 bp, Artificial Sequence
  MGH2 light chain variant 1
tacatcgtgatgacccagagccccctgagcctgcccgtgaccctgggccagcccgccagcatcagctgcagaagcagccagagcctggtgtacagcgacggcaacacctacctgaactggtaccagcagagacccggccagagccccagaagactgatctacaaggtgagcaacagagacagcggcgtgcccgacagattcagcggcagcggcagcggcaccgacttcaccctgaagatcagcagagtggaggccgaggacgtgggcgtgtactactgcatgcagggcacccactggtggaccttcggccagggcaccaaggtggagatcaagagaaccgtggccgcccccagcgtgttcatcttcccccccagcgacgagcagctgaagagcggcaccgccagcgtggtgtgcctgctgaacaacttctaccccagagaggccaaggtgcagtggaaggtggacaacgccctgcagagcggcaacagccaggagagcgtgaccgagcaggacagcaaggacagcacctacagcctgagcagcaccctgaccctgagcaaggccgactacgagaagcacaaggtgtacgcctgcgaggtgacccaccagggcctgagcagccccgtgaccaagagcttcaacagaggcgagtgc

SEQ ID NO: 62 - DNA, 654 bp, Artificial Sequence
  MGH2 light chain variant 2
tacattgttatgacccagagtcctctttcactgcctgtgacccttggccagcctgcctccatcagctgccggtccagccaatctctcgtgtactccgacggcaatacctacctgaactggtatcaacaacgacccggccagtcacccagacgcctgatctataaggtcagcaatcgggacagcggcgtcccggataggttctcaggttcaggttcaggcaccgatttcacgctgaaaattagtagagttgaggcagaagatgtcggcgtgtactactgtatgcagggtacccattggtggacctttgggcagggcacaaaagtagagattaagcggactgtggcagctccctcagtctttatatttcccccatccgatgagcagttgaaaagcgggaccgcatcagttgtgtgtctgttgaacaacttttaccctcgggaggccaaggtgcagtggaaggttgataacgctttacagtcaggcaattctcaggaaagtgtaacagaacaggattctaaggactcaacttatagcctctccagcaccctcacattgtcaaaggccgactatgagaagcacaaagtgtatgcgtgtgaggttacacatcagggcctgagctctccggtaacaaagtcttttaacaggggagagtgc

SEQ ID NO: 63 - DNA, 654 bp, Artificial Sequence
  MGH2 light chain variant 3
tacatagtaatgacgcagagtcctctgtccttaccagttacactgggccaacctgcatctatatcgtgtcgatcatctcagtccctcgtgtactcagatggaaatacgtatttgaactggtatcaacagcgtccgggacagagccctcgccgtttaatctacaaagttagtaaccgagacagtggcgttcctgaccgtttctcaggatcaggttccgggacagatttcaccttaaaaataagcagggttgaagctgaggacgtgggggtttattattgcatgcagggtacccactggtggacttttggacagggtacgaaggttgagatcaagcggaccgttgcagccccgtccgtctttattttccccccgtccgatgaacaattgaaatccggtacagcgtcagtagtatgcctcctgaacaatttttatcctcgggaagccaaggttcagtggaaagtggataacgcacttcagtccggcaacagtcaggagtctgtgaccgagcaagactctaaagactcaacctactctctttcctcgaccctgactctttcaaaggcggattatgagaagcacaaagtttatgcctgtgaagtaacacatcaaggcttgtcgtcaccggttaccaaatcgttcaacagaggggagtgc

SEQ ID NO: 64 - DNA, 24 bp, Artificial Sequence
  MGU10 CDRH1v1
ggcttcgccttcagcaactacggc

SEQ ID NO: 65 - DNA, 24 bp, Artificial Sequence
  MGU10 CDRH1v2
ggcttcgccttcagtaattacggc

SEQ ID NO: 66 - DNA, 24 bp, Artificial Sequence
  MGU10 CDRH1v3
ggctttgcgttcagtaattatggc

SEQ ID NO: 67 - DNA, 24 bp, Artificial Sequence
  MGU10 CDRH2v1
atctggcacgacggcagcctgaag

SEQ ID NO: 68 - DNA, 24 bp, Artificial Sequence
  MGU10 CDRH2v2
atttggcatgacggctctttgaaa

SEQ ID NO: 69 - DNA, 24 bp, Artificial Sequence
  MGU10 CDRH2v3
atttggcatgacggaagcttgaag

SEQ ID NO: 70 - DNA, 42 bp, Artificial Sequence
  MGU10 CDRH3v1
accgtgtggtacctggagacccccgacgacggcttcgacatc

SEQ ID NO: 71 - DNA, 42 bp, Artificial Sequence
  MGU10 CDRH3v2
accgtgtggtatctcgagacacccgatgatggcttcgacatc

SEQ ID NO: 72 - DNA, 42 bp, Artificial Sequence
  MGU10 CDRH3v3
accgtttggtacttggaaacgccggacgacgggtttgatatc

SEQ ID NO: 73 - DNA, 21 bp, Artificial Sequence
  MGU10 CDRL1v1
cacggccacaccagcaaggcc

SEQ ID NO: 74 - DNA, 21 bp, Artificial Sequence
  MGU10 CDRL1v2
cacgggcacacaagcaaggcc

SEQ ID NO: 75 - DNA, 21 bp, Artificial Sequence
  MGU10 CDRL1v3
cacggccacacgtcgaaggct

SEQ ID NO: 76 - DNA, 21 bp, Artificial Sequence
  MGU10 CDRL2v1
gtcaattccgacggatctcat

SEQ ID NO: 77 - DNA, 21 bp, Artificial Sequence
  MGU10 CDRL2v2
gtcaactccgacggcagtcat

SEQ ID NO: 78 - DNA, 21 bp, Artificial Sequence
  MGU10 CDRL2v3
gtgaacagcgacggcagccac

SEQ ID NO: 79 - DNA, 27 bp, Artificial Sequence
  MGU10 CDRL3v1
caggcctgggacagcggcatctgggtg

SEQ ID NO: 80 - DNA, 27 bp, Artificial Sequence
  MGU10 CDRL3v2
caggcctgggactcaggcatatgggta

SEQ ID NO: 81 - DNA, 27 bp, Artificial Sequence
  MGU10 CDRL3v3
caggcctgggactcaggtatttgggta

SEQ ID NO: 82 - DNA, 24 bp, Artificial Sequence
  MGH2 CDRH1v1
ggcttcagcttcagcagctacgcc

SEQ ID NO: 83 - DNA, 24 bp, Artificial Sequence
  MGH2 CDRH1v2
ggattctcattctcttcctacgcc

SEQ ID NO: 84 - DNA, 24 bp, Artificial Sequence
  MGH2 CDRH1v3
gggtttagcttctcttcctatgcg

SEQ ID NO: 85 - DNA, 24 bp, Artificial Sequence
  MGH2 CDRH2v1
actcggtatgacggttcgaataag

SEQ ID NO: 86 - DNA, 24 bp, Artificial Sequence
  MGH2 CDRH2v2
acaaggtatgatggttcaaacaag

SEQ ID NO: 87 - DNA, 24 bp, Artificial Sequence
  MGH2 CDRH2v3
accagatacgacggcagcaacaag

SEQ ID NO: 88 - DNA, 42 bp, Artificial Sequence
  MGH2 CDRH3v1
gccaaggtgggcgacggcaccgtggccggcaccatcgactac

SEQ ID NO: 89 - DNA, 42 bp, Artificial Sequence
  MGH2 CDRH3v2
gctaaggttggggacggcaccgttgccggcacaatcgactat

SEQ ID NO: 90 - DNA, 42 bp, Artificial Sequence
  MGH2 CDRH3v3
gctaaggtaggcgatggtacggtagcaggcacgatagattac

SEQ ID NO: 91 - DNA, 33 bp, Artificial Sequence
  MGH2 CDRL1v1
cagagcctggtgtacagcgacggcaacacctac

SEQ ID NO: 92 - DNA, 33 bp, Artificial Sequence
  MGH2 CDRL1v2
caatctctcgtgtactccgacggcaatacctac

SEQ ID NO: 93 - DNA, 33 bp, Artificial Sequence
  MGH2 CDRL1v3
cagtccctcgtgtactcagatggaaatacgtat

SEQ ID NO: 94-96 - skipped (3 entries)

SEQ ID NO: 97 - DNA, 24 bp, Artificial Sequence
  MGH2 CDRL3v1
atgcagggcacccactggtggacc

SEQ ID NO: 98 - DNA, 24 bp, Artificial Sequence
  MGH2 CDRL3v2
atgcagggtacccattggtggacc

SEQ ID NO: 99 - DNA, 24 bp, Artificial Sequence
  MGH2 CDRL3v3
atgcagggtacccactggtggact

SEQ ID NO: 100 - protein, 451 aa, Artificial Sequence
  MGU10v2_LS heavy chain
QVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDEGFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 101 - protein, 451 aa, Artificial Sequence
  MGU10_LS heavy chain
QVQLVESGGGVVQPGRSLRLSCAASGFAFSNYGMNWVRQAPGKGLEWVAVIWHDGSLKYYTQSVKGRFTISRDNAKNTLFLQMDSLSADDTAMYYCTVWYLETPDDGFDIWGRGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 102 - protein, 451 aa, Artificial Sequence
  MGH2/MGH2v1_LS heavy chain
QVQLVESGGGVVQPGGSLRLSCTASGFSFSSYAMHWVRQAPGKGLEWVAYTRYDGSNKFYLDSVQGRFTISRDNSKNTLYLEMDSLRLEDTAVYFCAKVGDGTVAGTIDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 103 - protein, 330 aa, Artificial Sequence
  heavy chain constant region LS
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVLHEALHSHYTQKSLSLSPGK

SEQ ID NO: 104 - protein, 121 aa, Artificial Sequence
  MGU10v8 VH
QVQLVESGGGVVQPGRSLRLSCAASGFAFSNYGMNWVRQAPGKGLEWVAVIWHDGSLKYYTQSVKGRFTISRDNAKNTLFLQMDSLSAEDTAMYYCTVWYLETPDEGFDIWGRGTMVTVSS

SEQ ID NO: 105 - protein, 19 aa, Artificial Sequence
  NPDP19-peptide
KQPADGNPDPNANPNVDPN

SEQ ID NO: 106 - DNA, 386 bp, Artificial Sequence
  MGU10v8 VH
accggtgtacattctcaggtgcagctggtggagtccggaggaggagtggtgcagccaggcaggagcctgaggctgtcttgcgctgcttccggattcgcctttagcaactacggcatgaattgggtgaggcaggctcctggcaagggactggagtgggtggctgtgatctggcacgacggcagcctgaagtactatacacagtctgtgaagggcagattcaccatctctcgcgataacgctaagaatacactgtttctgcagatggactctctgtccgccgaggataccgctatgtactattgtacagtgtggtatctggagaccccagacgagggcttcgatatctggggcagaggcaccatggtgacagtgtccagcgcgtcgac

SEQ ID NO: 107 - protein, 19 aa, Artificial Sequence
  Chimeric hinge sequence
ESKYGPPCPPCPAPPVAGP

(19) United States
(12) Patent Application Publication
(10) Pub. No.: US 2023/0084102 A1
(43) Pub. Date: Mar. 16, 2023

(54) ANTIBODIES BINDING TO PLASMODIUM CIRCUMSPOROZOITE PROTEIN AND USES THEREOF

(71) Applicants: HUMABS BIOMED SA, Bellinzona (CH); VIR BIOTECHNOLOGY, INC., San Francisco, CA (US)

(72) Inventors: Davide CORTI, Bellinzona (CH); Luca PICCOLI, Bellinzona (CH); Katja FINK, Bellinzona (CH); Elisabetta CAMERONI, Bellinzona (CH)

(21) Appl. No.: 17/607,631

(22) PCT Filed: Apr. 30, 2020
(86) PCT No.: PCT/EP2020/062167

§ 371 Date: Oct. 29, 2021

(30) Foreign Application Priority Data
Apr. 30, 2019 (EP) PCT/EP2019/061135

(51) Int. Cl. C07K 16/20; A61P 33/06

(52) U.S. Cl. CPC C07K 16/205; A61P 33/06; A61K 2039/505

(57) ABSTRACT

The present invention provides antibodies targeting Plasmodium sporozoites, in particular plasmodium circumsporozoite protein. The invention also provides nucleic acids that encode such antibodies. In addition, the invention provides the use of the antibodies of the invention in prophylaxis and treatment malaria.

The present invention relates to the field of malaria medication, in particular to malaria vaccination and to antibodies binding to plasmodium sporozoites, in particular to plasmodium circumsporozoite protein.

Malaria is one of the most severe public health problems worldwide. Malaria is caused by parasitic protozoans of the genus Plasmodium. The genus Plasmodium includes about 200 species with P. falciparum, P. vivax, P. ovale, and P. malariae together accounting for nearly all human infections with Plasmodium species. Among those Plasmodium species, P. falciparum accounts for the overwhelming majority of malaria deaths. Malaria symptoms typically include fever, feeling tired, vomiting, and headaches. In severe cases it can cause yellow skin, seizures, coma, or death.

Malaria is a mosquito-borne disease, which is most commonly transmitted by an infected female Anopheles mosquito. For example, during Plasmodium falciparum infection, the female Anopheles mosquito injects a small number of sporozoites (˜10-100) into the skin of a vertebrate host, after which they travel to the liver to invade hepatocytes (Crompton et al. (2014) Annu Rev Immuno/32, 157-187). In hepatocytes the sporozoites reproduce asexually (tissue schizogony) and mature into schizonts, which rupture to release merozoites. Merozoites infect red blood cells, ring stage trophozoites mature into schizonts, which rupture releasing merozoites. Other merozoites develop into sexual erythrocytic stages (gametocytes). When a mosquito bites an infected vertebrate host, gametocytes are taken up with the blood and mature in the mosquito gut. The male and female gametocytes fuse and form an ookinete —a fertilized, motile zygote. Ookinetes develop into new sporozoites that migrate to the insect's salivary glands to infect a new vertebrate host.

Malaria symptoms are caused by blood stage parasites. In contrast, sporozoites are not associated with clinical symptoms, however, in sporozoite and liver stages of the life cycle of Plasmodium parasite numbers in the host are low and their eradication can completely abrogate infection. Accordingly, the sporozoite and liver stages of the P. falciparum parasite represent key targets of current malaria medication candidates, as a medication that successfully protects against these stages would be able to prevent both malaria infection and transmission. Therefore, subunit vaccines based on circumsporozoite protein (CSP), such as RTS,S, are at the center of the malaria vaccine effort.

The Plasmodium circumsporozoite protein (CSP) is a secreted protein of the sporozoite stage of Plasmodium. CSP forms a dense coat on the surface of the parasite and has been hypothesized to mediate many of the initial interactions between the sporozoite and its two hosts (Menard R., 2000, Microbes Infect. 2:633-642; Sinnis P. and Nardin E., 2002, Sporozoite antigens: biology and immunology of the circumsporozoite protein and thrombospondin related anonymous protein. In Malaria Immunology. P. Perlmann and M. Troye-Blomberg, editors. S. Karger A G, Basel, Switzerland. 70-96). The structure and function of CSP is highly conserved across the various strains of Plasmodium that infect humans, non-human primates and rodents. The amino-acid sequence of CSP comprises an immunodominant central repeat region, that is diverse across Plasmodium species (NANP-repeat region in case of P. falciparum). Flanking the repeats are two conserved motifs at the N- and C-termini, namely region 1, a 5-aa sequence at the N terminus of the repeats, and a known cell-adhesive motif C-terminal to the repeats termed the type I thrombospondin repeat (TSR). Those conserved motifs are implicated in protein processing as the parasite travels from the mosquito to the mammalian vector.

CSP is known to play a crucial role in the migration of the sporozoites from the midgut walls of infected mosquitoes to the mosquito salivary glands. Additionally, CSP is involved in hepatocyte binding in the mammalian host with the N-terminus and central repeat region of CSP initially facilitate parasite binding. On the hepatocyte surface proteolytic cleavage at region 1 of the N-terminus exposes the adhesive domain of the C-terminus, thereby priming the parasites for invasion of the liver (Coppi et al. (2005) J Exp Med 201, 27-33).

At present, the most advanced malaria vaccine candidate is RTS,S (RTS,S/AS01; trade name Mosquirix), which is a recombinant protein-based malaria vaccine. RTS,S is a hybrid protein particle, formulated in a multi-component adjuvant named AS01. The RTS,S vaccine antigen consists of 19 NANP amino acid repeat units followed by the complete C-terminal domain minus the GPI anchor of the CS antigen, fused to the Hepatitis B virus S protein. Multisite clinical trials in sub-Saharan Africa have shown that RTS,S confers modest and short-lived protection against clinical malaria.

Another factor that has complicated the development of malaria medications is the difficulty in identifying robust correlates of protection. Antibodies have been shown to inhibit sporozoite invasion of hepatocytes in in vitro functional assays, but their role in the protection of malaria-vaccinated individuals remains unclear.

Recently, however, very potent anti-malaria antibodies were described, which are specific for Plasmodium falciparum circumsporozoite protein (CSP) (Tan J, Sack B K, Oyen D, et al. A public antibody lineage that potently inhibits malaria infection through dual binding to the circumsporozoite protein. Nat Med. 2018; 24(4):401-407. doi:10.1038/nm.4513). The authors of this study showed that the most potent antibodies—including antibodies “MGU10” and “MGH2”— simultaneously target epitopes in (i) the NANP-repeat region of CSP and (ii) an N-terminal region of CSP covering the junction between the N-terminal domain and the NANP-repeats. Moreover, this study showed that the extreme potency of those antibodies was due to their dual specificity, while antibodies targeting only one of the CSP epitopes were typically less potent.

In view of the above, it is the object of the present invention to overcome the drawbacks of prior art outlined above. In particular, it is the object of the present invention to provide anti-malaria antibodies, which bind to both, (i) the NANP-repeat region of CSP and (ii) an N-terminal region of CSP covering the junction between the N-terminal domain and the NANP-repeats, with high affinity.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the term “comprise”, and variations such as “comprises” and “comprising”, will be understood to imply the inclusion of a stated member, integer or step but not the exclusion of any other non-stated member, integer or step. The term “consist of” is a particular embodiment of the term “comprise”, wherein any other non-stated member, integer or step is excluded. In the context of the present invention, the term “comprise” encompasses the term “consist of”. The term “comprising” thus encompasses “including” as well as “consisting” e.g., a composition “comprising” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%, for example, x±5%, or x±7%, or x±10%, or x±12%, or x±15%, or x±20%.

The term “disease” as used herein is intended to be generally synonymous, and is used interchangeably with, the terms “disorder” and “condition” (as in medical condition), in that all reflect an abnormal condition of the human or animal body or of one of its parts that impairs normal functioning, is typically manifested by distinguishing signs and symptoms, and causes the human or animal to have a reduced duration or quality of life.

As used herein, reference to “treatment” of a subject or patient is intended to include prevention, prophylaxis, attenuation, amelioration and therapy. The terms “subject” or “patient” are used interchangeably herein to mean all mammals including humans. Examples of subjects include humans, cows, dogs, cats, horses, goats, sheep, pigs, and rabbits. In some embodiments, the patient is a human.

Doses are often expressed in relation to the bodyweight. Thus, a dose which is expressed as [g, mg, or other unit]/kg (or g, mg etc.) usually refers to [g, mg, or other unit] “per kg (or g, mg etc.) bodyweight”, even if the term “bodyweight” is not explicitly mentioned.

The term “binding” and similar reference usually means “specifically binding”, which does not encompass non-specific sticking.

As used herein, the term “antibody” encompasses various forms of antibodies including, without being limited to, whole antibodies, antibody fragments (such as antigen binding fragments), human antibodies, chimeric antibodies, humanized antibodies, recombinant antibodies and genetically engineered antibodies (variant or mutant antibodies) as long as the characteristic properties according to the invention are retained. In some embodiments, the antibody is a human antibody. In some embodiments, the antibody is a monoclonal antibody. For example, the antibody is a human monoclonal antibody.

As described above, the term “antibody” generally also includes antibody fragments. Fragments of the antibodies may retain the antigen-binding activity of the antibodies. Such fragments are referred to as “antigen-binding fragments”. Antigen-binding fragments include, but are not limited to, single chain antibodies, Fab, Fab′, F(ab′)2, Fv or scFv. Fragments of the antibodies can be obtained from the antibodies by methods that include digestion with enzymes, such as pepsin or papain, and/or by cleavage of disulfide bonds by chemical reduction. Alternatively, fragments of the antibodies can be obtained by recombinant means, for example by cloning and expressing a part (fragment) of the sequences of the heavy and/or light chain. The invention also encompasses single-chain Fv fragments (scFv) derived from the heavy and light chains of an antibody of the invention. For example, the invention includes a scFv comprising the CDRs from an antibody of the invention. Also included are heavy or light chain monomers and dimers, single domain heavy chain antibodies, single domain light chain antibodies, as well as single chain antibodies, e.g., single chain Fv in which the heavy and light chain variable domains are joined by a peptide linker. Antibody fragments of the invention may be contained in a variety of structures known to the person skilled in the art. In addition, the sequences of the invention may be a component of multispecific molecules in which the sequences of the invention target the epitopes of the invention and other regions of the molecule bind to other targets. Although the specification, including the claims, may, in some places, refer explicitly to antigen binding fragment(s), antibody fragment(s), variant(s) and/or derivative(s) of antibodies, it is understood that the term “antibody” includes all categories of antibodies, namely, antigen binding fragment(s), antibody fragment(s), variant(s) and derivative(s) of antibodies.

Human antibodies are well-known in the state of the art (van Dijk, M. A., and van de Winkel, J. G., Curr. Opin. Chem. Biol. 5 (2001) 368-374). Human antibodies can also be produced in transgenic animals (e.g., mice) that are capable, upon immunization, of producing a full repertoire or a selection of human antibodies in the absence of endogenous immunoglobulin production. Transfer of the human germ-line immunoglobulin gene array in such germ-line mutant mice will result in the production of human antibodies upon antigen challenge (see, e.g., Jakobovits, A., et al., Proc. Natl. Acad. Sci. USA 90 (1993) 2551-2555; Jakobovits, A., et al., Nature 362 (1993) 255-258; Bruggemann, M., et al., Year Immunol, 7 (1993) 3340). Human antibodies can also be produced in phage display libraries (Hoogenboom, H. R., and Winter, G., J. Mot Biol. 227 (1992) 381-388; Marks, J. D., et al., J. Mol. Biol. 222 (1991) 581-597). The techniques of Cole et al. and Boerner et al. are also available for the preparation of human monoclonal antibodies (Cole et al., Monoclonal Antibodies and Cancer Therapy, Alan R. Liss, p. 77 (1985); and Boerner, P., et al., J. Immunol. 147 (1991) 86-95). In some embodiments, human monoclonal antibodies are prepared by using improved EBV-B cell immortalization as described in Traggiai E, Becker S, Subbarao K, Kolesnikova L, Uematsu Y, Gismondo M R, Murphy B R, Rappuoli R, Lanzavecchia A. (2004): An efficient method to make human monoclonal antibodies from memory B cells: potent neutralization of SARS coronavirus. Nat Med. 10(8):871-5. As used herein, the term “variable region” (variable region of a light chain (V_{L}), variable region of a heavy chain (VH)) denotes each of the pair of light and heavy chains which is involved directly in binding the antibody to the antigen.

Antibodies of the invention can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1. Antibodies of the invention may have a κ or a λ light chain. In some embodiments, the antibody is of IgG1 type and has a κ light chain.

Antibodies according to the present invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies according to the present invention may be immunogenic in human and/or in non-human (or heterologous) hosts e.g., in mice. For example, the antibodies may have an idiotope that is immunogenic in non-human hosts, but not in a human host. Antibodies of the invention for human use include those that cannot be easily isolated from hosts such as mice, goats, rabbits, rats, non-primate mammals, etc. and cannot generally be obtained by humanization or from xeno-mice.

As used herein, a “neutralizing antibody” is one that can neutralize, i.e., prevent, inhibit, reduce, impede or interfere with, the ability of a pathogen to initiate and/or perpetuate an infection in a host. The terms “neutralizing antibody” and “an antibody that neutralizes” or “antibodies that neutralize” are used interchangeably herein. These antibodies can be used alone, or in combination, as prophylactic or therapeutic agents upon appropriate formulation, in association with active vaccination, as a diagnostic tool, or as a production tool as described herein.

As used herein, the term “mutation” relates to a change in the nucleic acid sequence and/or in the amino acid sequence in comparison to a reference sequence, e.g. a corresponding genomic sequence. A mutation, e.g. in comparison to a genomic sequence, may be, for example, a (naturally occurring) somatic mutation, a spontaneous mutation, an induced mutation, e.g. induced by enzymes, chemicals or radiation, or a mutation obtained by site-directed mutagenesis (molecular biology methods for making specific and intentional changes in the nucleic acid sequence and/or in the amino acid sequence). Thus, the terms “mutation” or “mutating” shall be understood to also include physically making a mutation, e.g. in a nucleic acid sequence or in an amino acid sequence. A mutation includes substitution, deletion and insertion of one or more nucleotides or amino acids as well as inversion of several successive nucleotides or amino acids. To achieve a mutation in an amino acid sequence, a mutation may be introduced into the nucleotide sequence encoding said amino acid sequence in order to express a (recombinant) mutated polypeptide. A mutation may be achieved e.g., by altering, e.g., by site-directed mutagenesis, a codon of a nucleic acid molecule encoding one amino acid to result in a codon encoding a different amino acid, or by synthesizing a sequence variant, e.g., by knowing the nucleotide sequence of a nucleic acid molecule encoding a polypeptide and by designing the synthesis of a nucleic acid molecule comprising a nucleotide sequence encoding a variant of the polypeptide without the need for mutating one or more nucleotides of a nucleic acid molecule.

Several documents are cited throughout the text of this specification. Each of the documents cited herein (including all patents, patent applications, scientific publications, manufacturer's specifications, instructions, etc.), whether supra or infra, are hereby incorporated by reference in their entirety. Nothing herein is to be construed as an admission that the invention is not entitled to antedate such disclosure by virtue of prior invention.

It is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Antibodies and Antigen-Binding Fragments Thereof

Recently, very potent anti-malaria antibodies were described, which are specific for Plasmodium falciparum circumsporozoite protein (CSP) (Tan J, Sack B K, Oyen D, et al. A public antibody lineage that potently inhibits malaria infection through dual binding to the circumsporozoite protein. Nat Med. 2018; 24(4):401-407. doi:10.1038/nm.4513). The authors of this study showed that the most potent antibodies simultaneously target epitopes in (i) the NANP-repeat region of CSP and (ii) an N-terminal region of CSP covering the junction between the N-terminal domain and the NANP-repeats. Moreover, this study showed that the extreme potency of the antibodies was due to their dual specificity, while antibodies targeting only one of the CSP epitopes were typically less potent. The most potent dual-specific antibodies described in this study include antibodies “MGU10” and “MGH2”.

Based thereon, the present inventors designed sequence variants of antibodies MGU10 and MGH2 by artificially introducing mutations in the CDR or framework regions. Accordingly, antibodies MGU10 and MGH2 may be referred to herein as “parental” antibodies, while the antibodies of the present invention represent sequence variants or “variant” antibodies of said “parental” antibodies. The variant antibodies were then tested for their dual specificity as described in Tan et al. (Tan), Sack B K, Oyen D, et al. A public antibody lineage that potently inhibits malaria infection through dual binding to the circumsporozoite protein. Nat Med. 2018; 24(4):401-407. doi:10.1038/nm.4513). The variant antibodies of the present invention show a surprisingly high affinity for the target epitopes in CSP (the NANP-repeat region of CSP and the N-terminal region of CSP covering the junction between the N-terminal domain and the NANP-repeats).

In a first aspect the present invention provides an (isolated) antibody, or an antigen-binding fragment thereof, comprising (i) the heavy chain CDR1, CDR2, and CDR3 sequences as set forth in SEQ ID NO: 1, SEQ ID NO: 2, and SEQ ID NO: 12, respectively, and the light chain CDR1, CDR2, and CDR3 sequences as set forth in SEQ ID NO: 4, SEQ ID NO: 5, and SEQ ID NO: 6, respectively; or (ii) the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in SEQ ID NO: 4, SEQ ID NO: 5, and SEQ ID NO: 14, respectively; or (iii) the heavy chain CDR1, CDR2, and CDR3 sequences as set forth in SEQ ID NO: 1, SEQ ID NO: 2, and SEQ ID NO: 12, respectively, and the light chain CDR1, CDR2, and CDR3 sequences as set forth in SEQ ID NO: 4, SEQ ID NO: 5, and SEQ ID NO: 14, respectively; or (iv) the heavy chain CDR1, CDR2, and CDR3 sequences as set forth in SEQ ID NO: 17, SEQ ID NO: 18, and SEQ ID NO: 19, respectively, and the light chain CDR1, CDR2, and CDR3 sequences as set forth in SEQ ID NO: 20, SEQ ID NO: 21, and SEQ ID NO: 28, respectively; or (v) the heavy chain CDR1, CDR2, and CDR3 sequences as set forth in SEQ ID NO: 17, SEQ ID NO: 18, and SEQ ID NO: 19, respectively, and the light chain CDR1, CDR2, and CDR3 sequences as set forth in SEQ ID NO: 20, SEQ ID NO: 22, and SEQ ID NO: 28, respectively.

In general, the antibody, or an antigen-binding fragment thereof, according to the present invention, typically comprises (at least) three complementarity determining regions (CDRs) on a heavy chain and (at least) three CDRs on a light chain. In general, complementarity determining regions (CDRs) are the hypervariable regions present in heavy chain variable domains and light chain variable domains. Typically, the CDRs of a heavy chain and the connected light chain of an antibody together form the antigen receptor. Usually, the three CDRs (CDR1, CDR2, and CDR3) are arranged non-consecutively in the variable domain. Since antigen receptors are typically composed of two variable domains (on two different polypeptide chains, i.e. heavy and light chain: heavy chain variable region (VH) and light chain variable region (VL)), there are typically six CDRs for each antigen receptor (heavy chain: CDRH1, CDRH2, and CDRH3; light chain: CDRL1, CDRL2, and CDRL3). A classical single antibody molecule usually has two antigen receptors and therefore contains twelve CDRs. The CDRs on the heavy and/or light chain may be separated by framework regions, whereby a framework region (FR) is a region in the variable domain which is less “variable” than the CDR. For example, a chain (or each chain, respectively) may be composed of four framework regions, separated by three CDR's.

The sequences of the heavy chains and light chains of exemplary antibodies of the invention, comprising three different CDRs on the heavy chain and three different CDRs on the light chain were determined. The position of the CDR amino acids are defined according to the IMGT numbering system (IMGT: http://www.imgt.org/; cf. Lefranc, M.-P. et al. (2009) Nucleic Acids Res. 37, D1006-D1012).

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 13 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 16 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 11 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 13 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 16 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 70% or more (i.e. 71%, 72%, 73%, 74%, 75%, 76%, 77%, 78%, 79%, 80%, 8M), 82%, 83%, 84%, 85%, 86%, 87%, 88%, 89%, 90%, 9M), 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 24 and a light chain variable region comprising the amino acid sequence having 70% or more (i.e. 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 29, wherein the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21 or 22, and SEQ ID NO: 28, respectively) are maintained.

Sequence identity is usually calculated with regard to the full length of the reference sequence (i.e. the sequence recited in the application). Percentage identity, as referred to herein, can be determined, for example, using BLAST using the default parameters specified by the NCBI (the National Center for Biotechnology Information; http://www.ncbi.nlm.nih.gov/) [Blosum 62 matrix; gap open penalty=11 and gap extension penalty=1].

A “sequence variant” has an altered sequence in which one or more of the amino acids in the reference sequence is/are deleted or substituted, and/or one or more amino acids is/are inserted into the sequence of the reference amino acid sequence. As a result of the alterations, the amino acid sequence variant has an amino acid sequence which is at least 70% identical to the reference sequence. Variant sequences which are at least 70% identical have no more than 30 alterations, i.e. any combination of deletions, insertions or substitutions, per 100 amino acids of the reference sequence.

In general, while it is possible to have non-conservative amino acid substitutions, the substitutions are usually conservative amino acid substitutions, in which the substituted amino acid has similar structural or chemical properties with the corresponding amino acid in the reference sequence. By way of example, conservative amino acid substitutions involve substitution of one aliphatic or hydrophobic amino acids, e.g. alanine, valine, leucine and isoleucine, with another; substitution of one hydoxyl-containing amino acid, e.g. serine and threonine, with another; substitution of one acidic residue, e.g. glutamic acid or aspartic acid, with another; replacement of one amide-containing residue, e.g. asparagine and glutamine, with another; replacement of one aromatic residue, e.g. phenylalanine and tyrosine, with another; replacement of one basic residue, e.g. lysine, arginine and histidine, with another; and replacement of one small amino acid, e.g., alanine, serine, threonine, methionine, and glycine, with another.

Amino acid sequence insertions include amino- and/or carboxyl-terminal fusions ranging in length from one residue to polypeptides containing a hundred or more residues, as well as intrasequence insertions of single or multiple amino acid residues. Examples of terminal insertions include the fusion to the N- or C-terminus of an amino acid sequence to a reporter molecule or an enzyme.

In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97/s, 98%, 99% or more) identity to SEQ ID NO: 13 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91′)/(c), 92%, 93%, 94%, 95%, 96%, 97%, 98%, 99% or more) identity to SEQ ID NO: 16 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 9M), 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 11 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 13 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 16 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 24 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 29, wherein the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21 or 22, and SEQ ID NO: 28, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 75% or more (i.e. 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 13 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 16 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 11 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 13 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97/s, 98%, 99% or more) identity to SEQ ID NO: 16 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 24 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 29, wherein the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21 or 22, and SEQ ID NO: 28, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 80% or more (i.e. 81%, 82%, 83%, 84%, 85%, 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 13 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 7 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 16 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 11 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 13 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 16 and a light chain variable region comprising the amino acid sequence having 85% or more (i.e. 86%, 87%, 88%, 89%, 90%, 91°/o, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 24 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 29, wherein the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21 or 22, and SEQ ID NO: 28, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 85% or more (i.e. 86%, 87%, 88%, 89%, 90%, 91%, 92%, 93%, 94%, 95%, 96%, 97%, 98%, 99% or more) identity to SEQ ID NO: 104 and a light chain variable region comprising the amino acid sequence having 85% or more (i.e. 86%, 87%, 88%, 89%, 90%, 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 13 and a light chain variable region comprising the amino acid sequence having 90% or more (i.e. 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 7 and a light chain variable region comprising the amino acid sequence having 90% or more (i.e. 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 16 and a light chain variable region comprising the amino acid sequence having 90% or more (i.e. 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 11 and a light chain variable region comprising the amino acid sequence having 90% or more (i.e. 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 13 and a light chain variable region comprising the amino acid sequence having 90% or more (i.e. 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 16 and a light chain variable region comprising the amino acid sequence having 90% or more (i.e. 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 24 and a light chain variable region comprising the amino acid sequence having 90% or more (i.e. 91%, 92%, 93%, 94%, 95%, 96%, 97%, 98%, 99% or more) identity to SEQ ID NO: 29, wherein the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21 or 22, and SEQ ID NO: 28, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 104 and a light chain variable region comprising the amino acid sequence having 90% or more (i.e. 91%, 92%, 93%, 94%, 95%, 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 90% or more (i.e. 91%, 92%, 93%, 94%, 95%, 96%, 97%, 98%, 99% or more) identity to SEQ ID NO: 104 and a light chain variable region comprising the amino acid sequence having 90% or more (i.e. 91%, 92%, 93%, 94%, 95%, 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 13 and a light chain variable region comprising the amino acid sequence having 95% or more (i.e. 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 7 and a light chain variable region comprising the amino acid sequence having 95% or more (i.e. 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 16 and a light chain variable region comprising the amino acid sequence having 95% or more (i.e. 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 11 and a light chain variable region comprising the amino acid sequence having 95% or more (i.e. 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 3, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 13 and a light chain variable region comprising the amino acid sequence having 95% or more (i.e. 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained. In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 16 and a light chain variable region comprising the amino acid sequence having 95% or more (i.e. 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 14, respectively) are maintained.

In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 24 and a light chain variable region comprising the amino acid sequence having 95% or more (i.e. 96%, 97%, 98%, 99% or more) identity to SEQ ID NO: 29, wherein the CDR sequences as defined above (heavy chain CDR1, CDR2, and CDR3 sequences as set forth in SEQ ID NO: 17, SEQ ID NO: 18, and SEQ ID NO: 19, respectively; and light chain CDR1, CDR2, and CDR3 sequences as set forth in SEQ ID NO: 20, SEQ ID NO: 21 or 22, and SEQ ID NO: 28, respectively) are maintained. In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 104 and a light chain variable region comprising the amino acid sequence having 95% or more (i.e. 96%, 97%, 98%, 99% or more) identity to SEQ ID NO: 8, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained. In some embodiments, the antibody of the invention, or the antigen-binding fragment thereof, comprises a heavy chain variable region comprising an amino acid sequence having 95% or more (i.e. 96%, 97%, 98%, 99% or more) identity to SEQ ID NO: 104 and a light chain variable region comprising the amino acid sequence having 95% or more (i.e. 96%, 97%, 98%, 99% or more) identity to SEQ ID NO: 15, wherein the CDR sequences as defined above (heavy chain CDR1, CDR2, and CDR3 sequences as set forth in SEQ ID NO: 1, SEQ ID NO: 2, and SEQ ID NO: 12, respectively; and light chain CDR1, CDR2, and CDR3 sequences as set forth in SEQ ID NO: 4, SEQ ID NO: 5, and SEQ ID NO: 6, respectively) are maintained.

In some embodiments, the antibody, or the antigen-binding fragment thereof, comprises (i) a heavy chain variable region comprising an amino acid sequence as set forth in SEQ ID NO: 13 and a light chain variable region comprising the amino acid sequence as set forth in SEQ ID NO: 8; or (ii) a heavy chain variable region comprising an amino acid sequence as set forth in SEQ ID NO: 7 and a light chain variable region comprising the amino acid sequence as set forth in SEQ ID NO: 15; or (iii) a heavy chain variable region comprising an amino acid sequence as set forth in SEQ ID NO: 16 and a light chain variable region comprising the amino acid sequence as set forth in SEQ ID NO: 8; or (iv) a heavy chain variable region comprising an amino acid sequence as set forth in SEQ ID NO: 11 and a light chain variable region comprising the amino acid sequence as set forth in SEQ ID NO: 15; or (v) a heavy chain variable region comprising an amino acid sequence as set forth in SEQ ID NO: 13 and a light chain variable region comprising the amino acid sequence as set forth in SEQ ID NO: 15; or (vi) a heavy chain variable region comprising an amino acid sequence as set forth in SEQ ID NO: 16 and a light chain variable region comprising the amino acid sequence as set forth in SEQ ID NO: 15; or (vii) a heavy chain variable region comprising an amino acid sequence as set forth in SEQ ID NO: 24 and a light chain variable region comprising the amino acid sequence as set forth in SEQ ID NO: 29; or (viii) a heavy chain variable region comprising an amino acid sequence as set forth in SEQ ID NO: 104 and a light chain variable region comprising the amino acid sequence as set forth in SEQ ID NO: 8; or (ix) a heavy chain variable region comprising an amino acid sequence as set forth in SEQ ID NO: 104 and a light chain variable region comprising the amino acid sequence as set forth in SEQ ID NO: 15.

The present invention also provides an antibody, or an antigen-binding fragment thereof, comprising (i) a heavy chain variable region comprising an amino acid sequence as set forth in SEQ ID NO: 11 and a light chain variable region comprising the amino acid sequence as set forth in SEQ ID NO: 8; or (ii) a heavy chain variable region comprising an amino acid sequence as set forth in SEQ ID NO: 13 and a light chain variable region comprising the amino acid sequence as set forth in SEQ ID NO: 8; or (iii) a heavy chain variable region comprising an amino acid sequence as set forth in SEQ ID NO: 7 and a light chain variable region comprising the amino acid sequence as set forth in SEQ ID NO: 15; or (iv) a heavy chain variable region comprising an amino acid sequence as set forth in SEQ ID NO: 16 and a light chain variable region comprising the amino acid sequence as set forth in SEQ ID NO: 8; or (v) a heavy chain variable region comprising an amino acid sequence as set forth in SEQ ID NO: 11 and a light chain variable region comprising the amino acid sequence as set forth in SEQ ID NO: 15; or (vi) a heavy chain variable region comprising an amino acid sequence as set forth in SEQ ID NO: 13 and a light chain variable region comprising the amino acid sequence as set forth in SEQ ID NO: 15; or (vii) a heavy chain variable region comprising an amino acid sequence as set forth in SEQ ID NO: 16 and a light chain variable region comprising the amino acid sequence as set forth in SEQ ID NO: 15; or (viii) a heavy chain variable region comprising an amino acid sequence as set forth in SEQ ID NO: 24 and a light chain variable region comprising the amino acid sequence as set forth in SEQ ID NO: 29; or (ix) a heavy chain variable region comprising an amino acid sequence as set forth in SEQ ID NO: 104 and a light chain variable region comprising the amino acid sequence as set forth in SEQ ID NO: 8; or (x) a heavy chain variable region comprising an amino acid sequence as set forth in SEQ ID NO: 104 and a light chain variable region comprising the amino acid sequence as set forth in SEQ ID NO: 15.

The CDR and VH/VL sequences of exemplified antibodies of the invention, namely antibodies MGU10variant1 (MGU10v1), MGU10variant2 (MGU10v2), MGU10variant3 (MGU10v3), MGU10variant4 (MGU10v4), MGU10variant5 (MGU10v5), MGU10variant6 (MGU10v6), MGU10variant7 (MGU10v7), MGU10variant8 (MGU10v8), MGU10variant9 (MGU10v9), and MGH2variant1 (MGH2v1), and their respective wild-type reference antibodies MGU10 and MGH2, are shown in Table 1 below.

TABLE 1
CDR and VH/VL sequences of exemplified antibodies of the invention
and their respective reference antibodies MGU10 and MGH2.

| Antibody | Heavy chain | | | | Light chain | | | |
|---|---|---|---|---|---|---|---|---|
| name | CDR1 | CDR2 | CDR3 | VH | CDR1 | CDR2 | CDR3 | VL |
| MGU10 | 1 | 2 | 3 | 7 | 4 | 5 | 6 | 8 |
| MGU10v1 | 1 | 2 | 3 | 11 | 4 | 5 | 6 | 8 |
| MGU10v2 | 1 | 2 | 12 | 13 | 4 | 5 | 6 | 8 |
| MGU10v3 | 1 | 2 | 3 | 7 | 4 | 5 | 14 | 15 |
| MGU10v4 | 1 | 2 | 12 | 16 | 4 | 5 | 6 | 8 |
| MGU10v5 | 1 | 2 | 3 | 11 | 4 | 5 | 14 | 15 |
| MGU10v6 | 1 | 2 | 12 | 13 | 4 | 5 | 14 | 15 |
| MGU10v7 | 1 | 2 | 12 | 16 | 4 | 5 | 14 | 15 |
| MGU10v8 | 1 | 2 | 12 | 104 | 4 | 5 | 6 | 8 |
| MGU10v9 | 1 | 2 | 12 | 104 | 4 | 5 | 14 | 15 |
| MGH2 | 17 | 18 | 19 | 24 | 20 | 21/22 | 23 | 25 |
| MGH2v1 | 17 | 18 | 19 | 24 | 20 | 21/22 | 28 | 29 |

In particular, the antibody of the invention, or an antigen-binding fragment thereof, binds (specifically) to Plasmodium falciparum sporozoites. The antibody, or the antigen-binding fragment thereof, may provide protection against Plasmodium (falciparum), in particular the antibody, or the antigen-binding fragment thereof, may inhibit or reduce (symptoms of) Plasmodium (falciparum) infection. Accordingly, the antibody of the invention, or an antigen-binding fragment thereof, may prevent, reduce, inhibit and/or neutralize infection with Plasmodium falciparum. More specifically, the antibody according to the present invention, or the antigen-binding fragment thereof, may (specifically) bind to Plasmodium circumsporozoite protein (CSP), such as the Plasmodium falciparum circumsporozoite protein (PfCSP) according to SEQ ID NO: 33.

In other words, the antibody according to the present invention, or the antigen-binding fragment thereof, may be able to recognize an epitope, in particular a CSP epitope. In some embodiments, the antibody of the invention, or an antigen-binding fragment thereof, binds (specifically) to the NRNP-repeat region of Plasmodium falciparum circumsporozoite protein (PfCSP). In some embodiments, the antibody of the invention, or an antigen-binding fragment thereof, binds (specifically) to the N-terminal region of Plasmodium falciparum circumsporozoite protein, which covers the junction between the N-terminal domain and the NANP-repeats of circumsporozoite protein. Typically, the antibody of the invention, or an antigen-binding fragment thereof, may be monospecific regarding its paratopes (i.e., the antibody or antigen-binding fragment may contain only one single kind/type of antigen-binding site(s)); all antigen-binding site(s) of the antibody or antigen-binding fragment may have the same CDR or VH/VL sequences)—but, at the same time, the antibody or antigen-binding fragment may be “dual-specific” regarding the target epitopes at CSP (i.e., the antibody or antigen-binding fragment can recognize two (or more) epitopes on CSP, in particular the two epitopes described herein). Accordingly, a single paratope of the antibodies of the invention, or antigen-binding fragments thereof, may be able to bind to both, the NANP-repeat region of PfCSP and the N-terminal region of PfCSP, which covers the junction between the N-terminal domain and the NANP-repeats of circumsporozoite protein. The NANP-repeat region of CSP is well-known to those skilled in the art. For example, the NANP-repeat region of CSP may have an amino acid sequence as set forth in SEQ ID NO: 34. For example, the N-terminal region of CSP, which covers the junction between the N-terminal domain and the NANP-repeats of circumsporozoite protein may have an amino acid sequence as set forth in SEQ ID NO: 35 or 105. Accordingly, the antibody according to the present invention, or the antigen-binding fragment thereof, may bind (specifically) to a peptide according to SEQ ID NO: 34 and/or to a peptide according to SEQ ID NO: 35 or 105.

Standard methods to assess binding of the antibody according to the present invention, or the antigen-binding fragment thereof, are known to those skilled in the art and include, for example, ELISA (enzyme-linked immunosorbent assay). An exemplary standard ELISA may be performed as follows: ELISA plates may be coated with a sufficient amount (e.g., 1 μg/ml of the protein/complex/particle to which binding of the antibody is to be tested. For example, for testing binding to CSP or an epitope thereof as outlined above, a CSP protein (e.g., SEQ ID NO: 33) and/or fragments/epitopes thereof (e.g., peptides of SEQ ID NO: 34 or 35/105) may be used. ELISA plates may be coated directly or indirectly (e.g., by coating plates first with avidin and incubating them later with biotinylated protein/complex/particle to which binding of the antibody is to be tested). After the first coating step (avidin or, directly, with the protein/complex/particle to which binding of the antibody is to be tested) plates may be blocked, e.g. with a 1% w/v solution of Bovine Serum Albumin (BSA) in PBS. Before the coated plates are incubated with the antibody to be tested, they may be washed. To determine, for example, EC₅₀-values, the plates are typically incubated with different concentrations of the antibody to be tested (“titration”). Antibody binding can be revealed, for example, using goat anti-human IgG, e.g. coupled to alkaline phosphatase. Plates may then be washed, the required substrate (e.g., p-NPP) may be added and plates may be read, e.g. at a wavelength of 405 nm to determine optical density values. The relative affinities of antibody binding may be determined by measuring the concentration of the antibody required to achieve 50% maximal binding at saturation (EC₅₀) The EC₅₀ values may be calculated by interpolation of binding curves fitted with a four-parameter nonlinear regression with a variable slope. A specific example of such an ELISA is described in Example 2, which may be performed (in essentially the same way) also with other antibodies or antigen-binding fragments.

In some embodiments, the antibody of the invention, or the antigen-binding fragment thereof, has an EC₅₀ value below 10³ ng/ml, e.g. below 200 or 100 ng/ml, for binding to CSP (e.g., SEQ ID NO: 33), such as to a fragment/epitope thereof (e.g., SEQ ID NO: 34 and/or 35/105). For example, the antibody of the invention, or the antigen-binding fragment thereof, may have an EC₅₀ value below 10³ ng/ml, e.g. below 200 or 100 ng/ml, for binding to a peptide of SEQ ID NO: 34 and for binding to a peptide to SEQ ID NO: 35 or 105. More specifically, the antibody of the invention, or the antigen-binding fragment thereof, may have an EC₅₀ value below 30 ng/ml for binding to a peptide of SEQ ID NO: 34 and for binding to a peptide to SEQ ID NO: 35 or 105. For example, the antibody of the invention, or the antigen-binding fragment thereof, may have an EC₅₀ value below 29 ng/ml (e.g., below 28 or 27 ng/ml) for binding to a peptide of SEQ ID NO: 34 and an EC₅₀ value below 26 ng/ml (e.g., below 23 or 21 ng/ml) for binding to a peptide to SEQ ID NO: 35 or 105.

In some embodiments, the antibody, or an antigen-binding fragment thereof, according to according to the present invention comprises a variable region of the heavy chain of the antibody, or of the antigen-binding fragment thereof, (VH), which is encoded by a nucleic acid comprising a gene (segment) of the VH3 gene family, such as the gene (segment) VH3-30.

To study and quantitate virus infectivity (or “neutralization”) in the laboratory the person skilled in the art knows various standard “neutralization assays”. For a neutralization assay animal viruses are typically propagated in cells and/or cell lines. For example, in a neutralization assay cultured cells may be incubated with a fixed amount of Plasmodium falciparum sporozoites in the presence (or absence) of the antibody to be tested. As a readout for example flow cytometry may be used. Alternatively, also other readouts are conceivable.

In some instances, the antibody of the invention, or an antigen-binding fragment thereof, may reduce gliding motility of Plasmodium sporozoites. Plasmodium sporozoites are transmitted by mosquito bites into the skin of their vertebrate host. Before sporozoites enter the blood stream, they move rapidly through the dermis, powered by an actomyosin system, using a form of locomotion referred to as “gliding motility”. Accordingly, sporozoite motility is a key prerequisite for parasite transmission and successful infection of the vertebrate host.

Gliding motility of sporozoites can be assessed by in in vitro assays, wherein the sporozoite is allowed to glide on a flat surface, e.g. on a glass surface. Gliding trails of sporozoites can be visualized by covering the surface with an anti-CSP antibody, which detects CSP shed by sporozoites during gliding. The anti-CSP antibody itself may be labelled (e.g. biotin) or a secondary labelled antibody against the anti-CSP antibody may be used to visualize the trails. For testing the effect of compounds on sporozoite gliding, sporozoites may be pre-incubated with test compounds before they are allowed to glide. Detailed protocols for gliding assays are known in the art and described, for example, in Example 4 or in Prinz, H. L., Sattler, J. M. & Frischknecht, F. (2017) Plasmodium Sporozoite Motility on Flat Substrates. Bio-protocol7, e2395. Moreover, ex vivo imaging technologies making use of human tissue may be employed to determine the gliding motility of sporozoites, e.g. as described in Winkel, B. M. F., de Korne, C. M., van Oosterom, M. N. et al. (2019) Quantification of wild-type and radiation attenuated Plasmodium falciparum sporozoite motility in human skin. Sci Rep 9, 13436. In some embodiments, the antibody of the invention, or the antigen-binding fragment thereof, reduces Plasmodium sporozoite gliding motility to a greater extent than its parental antibody MGU10 or MGH2, respectively. This may be easily assessed by directly comparing the effects of the parental antibody (MGU10 or MGH2) and its variant antibody (or antigen-binding fragment thereof) according to the present invention in the same gliding motility assay, e.g. as described in Example 4.

In some instances, the antibody of the invention, or an antigen-binding fragment thereof, may reduce cell traversal of Plasmodium sporozoites. As sporozoites move towards the liver, they can enter and exit host cells within transient vacuoles, a process known as cell traversal. Traversal allows the sporozoites to cross cellular barriers and evade the host immune response, thereby representing a key prerequisite for successful infection of the vertebrate host.

Cell traversal of sporozoites can be assessed by in in vitro assays, wherein sporozoites are incubated with host cells in a co-culture. For visualization, various (e.g., fluorescent) labels may be used, for example, in the co-culture or sporozoites may be pre-incubated with a label (e.g., as described in Example 5). Alternatively, genetically modified Plasmodium strains may be used, which express, e.g., fluorescent labels. For testing the effect of compounds on traversal, sporozoites may be pre-incubated with test compounds before they are co-cultured with host cells. Detailed protocols for sporozoite traversal assays are known in the art and described, for example, in Example 5; in Schleicher, T. R., Yang, J., Freudzon, M. et al. (2018) A mosquito salivary gland protein partially inhibits Plasmodium sporozoite cell traversal and transmission. Nat Commun 9, 2908; or in Sinnis, P., De La Vega, P., Coppi, A., Krzych, U., & Mota, M. M. (2013). Quantification of sporozoite invasion, migration, and development by microscopy and flow cytometry. Methods in molecular biology (Clifton, N.J.), 923, 385-400. In some embodiments, the antibody of the invention, or the antigen-binding fragment thereof, reduces Plasmodium sporozoite cell traversal to a greater extent than its parental antibody MGU10 or MGH2, respectively. This may be easily assessed by directly comparing the effects of the parental antibody (MGU10 or MGH2) and its variant antibody (or antigen-binding fragment thereof) according to the present invention in the same traversal assay, e.g. as described in Example 5.

In some instances, the antibody of the invention, or an antigen-binding fragment thereof, may reduce invasion and/or maturation of Plasmodium sporozoites. Sporozoite invasion of hepatocytes and subsequent maturation into exoerythrocytic forms is an essential step in the establishment of malaria infection.

Invasion and/or maturation of sporozoites can be assessed by in in vitro assays, wherein sporozoites are incubated with host cells (e.g., hepatocytes). For visualization, various (e.g., fluorescent) labels may be used (e.g., as described in Example 3). Alternatively, genetically modified Plasmodium strains may be used, which express, e.g., fluorescent labels. For testing the effect of compounds on invasion and/or maturation, sporozoites may be pre-incubated with test compounds before they are co-incubated with host cells. Detailed protocols for sporozoite invasion/maturation assays are known in the art and described, for example, in Example 3; in Kaushansky, A., Rezakhani, N., Mann, H. & Kappe, S. H. (2012) Development of a quantitative flow cytometry based assay to assess infection by Plasmodium falciparum sporozoites. Molecular and biochemical parasitology 183, 100-103; in Rodriguez-Galan A, Salman A M, Bowyer G, Collins K A, Longley R J, Brod F, Ulaszewska M, Ewer K J, Janse C J, Khan S M, Hafalla J C, Hill A V S, Spencer A J. (2017) An in vitro assay to measure antibody-mediated inhibition of P. berghei sporozoite invasion against P. falciparum antigens. Sci Rep 5; 7(1):17011; or in Sinnis, P., De La Vega, P., Coppi, A., Krzych, U., & Mota, M. M. (2013). Quantification of sporozoite invasion, migration, and development by microscopy and flow cytometry. Methods in molecular biology (Clifton, N. J.), 923, 385-400. In some embodiments, the antibody of the invention, or the antigen-binding fragment thereof, reduces Plasmodium sporozoite invasion/maturation to a greater extent than its parental antibody MGU10 or MGH2, respectively. This may be easily assessed by directly comparing the effects of the parental antibody (MGU10 or MGH2) and its variant antibody (or antigen-binding fragment thereof) according to the present invention in the same invasion/maturation assay, e.g. as described in Example 3.

In some embodiments, the antibody of the invention, or an antigen-binding fragment thereof, may exhibit increased stability as compared to its parental antibody MGU10 or MGH2, respectively. It is understood that for comparison, the variant antibody of the invention and its parental antibody are tested under the same conditions (i.e., side-by-side). For example, the antibody of the invention, or an antigen-binding fragment thereof, may exhibit increased stability as compared to its parental antibody MGU10 or MGH2, respectively, at a pH below 6, such as pH 5.5 or 5.6. This may be achieved, for example, by storing the antibodies in a buffer comprising 50 mM Na-Acetate and 50 mM NaCl, at pH 5.5. Moreover, the antibodies may be exposed to heat stress, e.g. about 40° C., for testing their stability. Stability test usually continue for at least several days, e.g., 2, 3, 4, 5, 6, 7, 8, 9, 10, 11, 12, 13, 14, 15, 16, 17, 18, 19, 20 or more days. In some instances, stability is tested over 14 or 15 days.

In some embodiments, the antibody of the invention is a human antibody. In some embodiments, the antibody of the invention is a monoclonal antibody. For example, the antibody of the invention is a human monoclonal antibody.

Antibodies of the invention can be of any isotype (e.g., IgA, IgG, IgM i.e. an α, γ or μ heavy chain). For example, the antibody is of the IgG type. Within the IgG isotype, antibodies may be IgG1, IgG2, IgG3 or IgG4 subclass, for example IgG1. Antibodies of the invention may have a κ or a λ light chain. In some embodiments, the antibody has a lambda or kappa light chain. In some embodiments, the antibody is of IgG1 type and has a lambda or kappa light chain.

In some embodiments, the antibody is of the human IgG1 type. The antibody may be of any allotype. The term “allotype” refers to the allelic variation found among the IgG subclasses. For example, the antibody may be of the G1 m1 (or G1m(a)) allotype, of the G1 m2 (or G1m(x)) allotype, of the G1m3 (or G1m(f)) allotype, and/or of the G1m17 (or Gm(z)) allotype. The G1m3 and G1m17 allotypes are located at the same position in the CH1 domain (position 214 according to EU numbering). G1 m3 corresponds to R214 (EU), while G1m17 corresponds to K214 (EU). The G1 m1 allotype is located in the CH3 domain (at positions 356 and 358 (EU)) and refers to the replacements E356D and M358L. The G1 m2 allotype refers to a replacement of the alanine in position 431 (EU) by a glycine. The G1 m1 allotype may be combined, for example, with the G1 m3 or the G1 m17 allotype. In some embodiments, the antibody is of the allotype G1 m3 with no G1 m1 (G1m3,−1). In some embodiments, the antibody is of the G1 m17,1 allotype. In some embodiments, the antibody is of the G1 m3,1 allotype. In some embodiments, the antibody is of the allotype G1m17 with no G1 m1 (G1 m17,−1). Optionally, these allotypes may be combined (or not combined) with the G1 m2, G1 m27 or G1 m28 allotype. For example, the antibody may be of the G1 m17,1,2 allotype.

In some embodiments, the antibody according to the present invention, or an antigen binding fragment thereof, comprises an Fc moiety. The Fc moiety may be derived from human origin, e.g. from human IgG1, IgG2, IgG3, and/or IgG4, such as human IgG1.

As used herein, the term “Fc moiety” refers to a sequence derived from the portion of an immunoglobulin heavy chain beginning in the hinge region just upstream of the papain cleavage site (e.g., residue 216 in native IgG, taking the first residue of heavy chain constant region to be 114) and ending at the C-terminus of the immunoglobulin heavy chain. Accordingly, an Fc moiety may be a complete Fc moiety or a portion (e.g., a domain) thereof.

A complete Fc moiety comprises at least a hinge domain, a CH2 domain, and a CH3 domain (e.g., EU amino acid positions 216-446). An additional lysine residue (K) is sometimes present at the extreme C-terminus of the Fc moiety, but is often cleaved from a mature antibody.

Each of the amino acid positions within an Fc moiety have been numbered herein according to the art-recognized EU numbering system of Kabat, see e.g., by Kabat et al., in “Sequences of Proteins of Immunological Interest”, U.S. Dept. Health and Human Services, 1983 and 1987. The EU index or EU index as in Kabat or EU numbering refers to the numbering of the EU antibody (Edelman G M, Cunningham B A, Gall W E, Gottlieb P D, Rutishauser U, Waxdal M J. The covalent structure of an entire gammaG immunoglobulin molecule. Proc Natl Acad Sci USA. 1969; 63(1):78-85; Kabat E.A., National Institutes of Health (U.S.) Office of the Director, “Sequences of Proteins of Immunological Interest”, 5^{th} edition, Bethesda, Md.: U.S. Dept. of Health and Human Services, Public Health Service, National Institutes of Health, 1991, hereby entirely incorporated by reference).

In some embodiments, in the context of the present invention an Fc moiety comprises at least one of: a hinge (e.g., upper, middle, and/or lower hinge region) domain, a CH2 domain, a CH3 domain, or a variant, portion, or fragment thereof. An Fc moiety may comprise at least a hinge domain, a CH2 domain or a CH3 domain. The Fc moiety may be a complete Fc moiety. The Fc moiety may also comprises one or more amino acid insertions, deletions, or substitutions relative to a naturally-occurring Fc moiety. For example, at least one of a hinge domain, CH2 domain or CH3 domain (or portion thereof) may be deleted. For example, an Fc moiety may comprise or consist of: (i) hinge domain (or portion thereof) fused to a CH2 domain (or portion thereof), (ii) a hinge domain (or portion thereof) fused to a CH3 domain (or portion thereof), (iii) a CH2 domain (or portion thereof) fused to a CH3 domain (or portion thereof), (iv) a hinge domain (or portion thereof), (v) a CH2 domain (or portion thereof), or (vi) a CH3 domain or portion thereof.

It will be understood by one of ordinary skill in the art that the Fc moiety may be modified such that it varies in amino acid sequence from the complete Fc moiety of a naturally occurring immunoglobulin molecule, while retaining at least one desirable function conferred by the naturally-occurring Fc moiety. Such functions include Fc receptor (FcR) binding, antibody half-life modulation, ADCC function, protein A binding, protein G binding, and complement binding. The portions of naturally occurring Fc moieties, which are responsible and/or essential for such functions are well known by those skilled in the art.

For example, to activate the complement cascade C1q binds to at least two molecules of IgG1 or one molecule of IgM, attached to the antigenic target (Ward, E. S., and Ghetie, V., Ther. Immunol. 2 (1995) 77-94). Burton, D. R., described (Mol. Immunol. 22 (1985) 161-206) that the heavy chain region comprising amino acid residues 318 to 337 is involved in complement fixation. Duncan, A. R., and Winter, G. (Nature 332 (1988) 738-740), using site directed mutagenesis, reported that Glu318, Lys320 and Lys322 form the binding site to C1q. The role of Glu318, Lys320 and Lys322 residues in the binding of C1q was confirmed by the ability of a short synthetic peptide containing these residues to inhibit complement mediated lysis.

For example, FcR binding can be mediated by the interaction of the Fc moiety (of an antibody) with Fc receptors (FcRs), which are specialized cell surface receptors on hematopoietic cells. Fc receptors belong to the immunoglobulin superfamily, and were shown to mediate both the removal of antibody-coated pathogens by phagocytosis of immune complexes, and the lysis of erythrocytes and various other cellular targets (e.g. tumor cells) coated with the corresponding antibody, via antibody dependent cell mediated cytotoxicity (ADCC; Van de Winkel, J. G., and Anderson, C. L., J. Leukoc. Biol. 49 (1991) 511-524). FcRs are defined by their specificity for immunoglobulin classes; Fc receptors for IgG antibodies are referred to as FcγR, for IgE as FcεR, for IgA as FcαR and so on and neonatal Fc receptors are referred to as FcRn. Fc receptor binding is described for example in Ravetch, J. V., and Kinet, J. P., Annu. Rev. Immunol. 9 (1991) 457-492; Capel, P. J., et al., Immunomethods 4 (1994) 25-34; de Haas, M., et al., J tab. Clin. Med. 126 (1995) 330-341; and Gessner, J. E., et al., Ann. Hematol. 76 (1998) 231-248.

Cross-linking of receptors by the Fc domain of native IgG antibodies (FcγR) triggers a wide variety of effector functions including phagocytosis, antibody-dependent cellular cytotoxicity, and release of inflammatory mediators, as well as immune complex clearance and regulation of antibody production. Therefore, the Fc moiety may provide cross-linking of receptors (FcγR). In humans, three classes of FcγR have been characterized, which are: (i) FcγRI (CD64), which binds monomeric IgG with high affinity and is expressed on macrophages, monocytes, neutrophils and eosinophils; (ii) FcγRII (CD32), which binds complexed IgG with medium to low affinity, is widely expressed, in particular on leukocytes, is known to be a central player in antibody-mediated immunity, and which can be divided into FcγRIIA, FcγRIIB and FcγRIIC, which perform different functions in the immune system, but bind with similar low affinity to the IgG-Fc, and the ectodomains of these receptors are highly homologuous; and (iii) FcγRIII (CD16), which binds IgG with medium to low affinity and exists as two types: FcγRIIIA found on NK cells, macrophages, eosinophils and some monocytes and T cells and mediating ADCC and FcγRIIIB, which is highly expressed on neutrophils. FcγRIIA is found on many cells involved in killing (e.g. macrophages, monocytes, neutrophils) and seems able to activate the killing process. FcγRIIB seems to play a role in inhibitory processes and is found on B-cells, macrophages and on mast cells and eosinophils. Importantly, 75% of all FcγRIIB is found in the liver (Ganesan, L. P. et al., 2012: FcγRIIb on liver sinusoidal endothelium clears small immune complexes. Journal of Immunology 189: 4981-4988). FcγRIIB is abundantly expressed on Liver Sinusoidal Endothelium, called LSEC, and in Kupffer cells in the liver and LSEC are the major site of small immune complexes clearance (Ganesan, L. P. et al., 2012: FcγRIIb on liver sinusoidal endothelium clears small immune complexes. Journal of Immunology 189: 4981-4988).

Accordingly, antibodies, and antigen binding fragments thereof, of the invention may be able to bind to FcγRIIb, for example antibodies comprising an Fc moiety for binding to FcγRIIb, in particular an Fc region, such as, for example IgG-type antibodies. Moreover, it is possible to engineer the Fc moiety to enhance FcγRIIB binding by introducing the mutations S267E and L328F as described by Chu, S. Y. et al., 2008: Inhibition of B cell receptor-mediated activation of primary human B cells by coengagement of CD19 and FcγRIIb with Fc-engineered antibodies. Molecular Immunology 45, 3926-3933. Thereby, the clearance of immune complexes can be enhanced (Chu, S., et al., 2014: Accelerated Clearance of IgE In Chimpanzees Is Mediated By Xmab7195, An Fc-Engineered Antibody With Enhanced Affinity For Inhibitory Receptor FcγRIIb. Am J Respir Crit, American Thoracic Society International Conference Abstracts). Accordingly, the antibodies, or antigen binding fragments thereof, of the invention may comprise an engineered Fc moiety with the mutations S267E and L328F, in particular as described by Chu, S. Y. et al., 2008: Inhibition of B cell receptor-mediated activation of primary human B cells by coengagement of CD19 and FcγRIIb with Fc-engineered antibodies. Molecular Immunology 45, 3926-3933.

On B-cells it seems to function to suppress further immunoglobulin production and isotype switching to say for example the IgE class. On macrophages, FcγRIIB acts to inhibit phagocytosis as mediated through FcγRIIA. On eosinophils and mast cells the b form may help to suppress activation of these cells through IgE binding to its separate receptor.

Regarding FcγRI binding, modification in native IgG of at least one of E233-G236, P238, D265, N297, A327 and P329 reduces binding to FcγRI. IgG2 residues at positions 233-236, substituted into IgG1 and IgG4, reduces binding to FcγRI by 10³-fold and eliminated the human monocyte response to antibody-sensitized red blood cells (Armour, K. L., et al. Eur. J. Immunol. 29 (1999) 2613-2624). Regarding FcγRII binding, reduced binding for FcγRIIA is found e.g. for IgG mutation of at least one of E233-G236, P238, D265, N297, A327, P329, D270, Q295, A327, R292 and K414. Regarding FcγR111 binding, reduced binding to FcγRIIIA is found e.g. for mutation of at least one of E233-G236, P238, D265, N297, A327, P329, D270, Q295, A327, S239, E269, E293, Y296, V303, A327, K338 and D376. Mapping of the binding sites on human IgG1 for Fc receptors, the above mentioned mutation sites and methods for measuring binding to FcγRI and FcγRIIA are described in Shields, R. L., et al., J. Biol. Chem. 276 (2001) 6591-6604. For example, a single (S239D or 1332E), double (S239D/I332E), and triple mutations (S239D/1332E/A330L) improved the affinity against human FcγRIIIa. Furthermore, the addition of the mutation G236A to S239D/I332E improved not only FcγRIIa:FcγRIIb ratio, but also enhanced binding to FcγRIIIa. Accordingly, the mutations G236A/S239D/A330L/1332E were described to enhance engagement of FcγRIIa and FcγRIIIa.

Regarding binding to the crucial FcγRII, two regions of native IgG Fc appear to be critical for interactions of FcγRIIs and IgGs, namely (i) the lower hinge site of IgG Fc, in particular amino acid residues L, L, G, G (234-237, EU numbering), and (ii) the adjacent region of the CH2 domain of IgG Fc, in particular a loop and strands in the upper CH2 domain adjacent to the lower hinge region, e.g. in a region of P331 (Wines, B. D., et al., J. Immunol. 2000; 164: 5313-5318). Moreover, FcγRI appears to bind to the same site on IgG Fc, whereas FcRn and Protein A bind to a different site on IgG Fc, which appears to be at the CH2-CH3 interface (Wines, B. D., et al., J. Immunol. 2000; 164: 5313-5318).

For example, the Fc moiety may comprise or consist of at least the portion of an Fc moiety that is known in the art to be required for FcRn binding or extended half-life. Alternatively or additionally, the Fc moiety of the antibody of the invention comprises at least the portion of known in the art to be required for Protein A binding and/or the Fc moiety of the antibody of the invention comprises at least the portion of an Fc molecule known in the art to be required for protein G binding. The Fc moiety may comprise at least the portion known in the art to be required for FcγR binding. As outlined above, an Fc moiety may thus at least comprise (i) the lower hinge site of native IgG Fc, in particular amino acid residues L, L, G, G (234-237, EU numbering), and (ii) the adjacent region of the CH2 domain of native IgG Fc, in particular a loop and strands in the upper CH2 domain adjacent to the lower hinge region, e.g. in a region of P331, for example a region of at least 3, 4, 5, 6, 7, 8, 9, or 10 consecutive amino acids in the upper CH2 domain of native IgG Fc around P331, e.g. between amino acids 320 and 340 (EU numbering) of native IgG Fc.

In some embodiments, the antibody, or antigen binding fragment thereof, according to the present invention comprises an Fc region. As used herein, the term “Fc region” refers to the portion of an immunoglobulin formed by two or more Fc moieties of antibody heavy chains. For example, the Fc region may be monomeric or “single-chain” Fc region (i.e., a scFc region). Single chain Fc regions are comprised of Fc moieties linked within a single polypeptide chain (e.g., encoded in a single contiguous nucleic acid sequence). Exemplary scFc regions are disclosed in WO 2008/143954 A2. The Fc region may be dimeric. A “dimeric Fc region” or “dcFc” refers to the dimer formed by the Fc moieties of two separate immunoglobulin heavy chains. The dimeric Fc region may be a homodimer of two identical Fc moieties (e.g., an Fc region of a naturally occurring immunoglobulin) or a heterodimer of two non-identical Fc moieties.

The Fc moieties of the Fc region may be of the same or different class and/or subclass. For example, the Fc moieties may be derived from an immunoglobulin (e.g., a human immunoglobulin) of an IgG1, IgG2, IgG3 or IgG4 subclass. The Fc moieties of the Fc region may be of the same class and subclass. However, the Fc region (or one or more Fc moieties of an Fc region) may also be chimeric, whereby a chimeric Fc region may comprise Fc moieties derived from different immunoglobulin classes and/or subclasses. For example, at least two of the Fc moieties of a dimeric or single-chain Fc region may be from different immunoglobulin classes and/or subclasses. Additionally or alternatively, the chimeric Fc regions may comprise one or more chimeric Fc moieties. For example, the chimeric Fc region or moiety may comprise one or more portions derived from an immunoglobulin of a first subclass (e.g., an IgG1, IgG2, or IgG3 subclass) while the remainder of the Fc region or moiety is of a different subclass. For example, an Fc region or moiety of an Fc polypeptide may comprise a CH2 and/or CH3 domain derived from an immunoglobulin of a first subclass (e.g., an IgG1, IgG2 or IgG4 subclass) and a hinge region from an immunoglobulin of a second subclass (e.g., an IgG3 subclass). For example, the Fc region or moiety may comprise a hinge and/or CH2 domain derived from an immunoglobulin of a first subclass (e.g., an IgG4 subclass) and a CH3 domain from an immunoglobulin of a second subclass (e.g., an IgG1, IgG2, or IgG3 subclass). For example, the chimeric Fc region may comprise an Fc moiety (e.g., a complete Fc moiety) from an immunoglobulin for a first subclass (e.g., an IgG4 subclass) and an Fc moiety from an immunoglobulin of a second subclass (e.g., an IgG1, IgG2 or IgG3 subclass). For example, the Fc region or moiety may comprise a CH2 domain from an IgG4 immunoglobulin and a CH3 domain from an IgG1 immunoglobulin. For example, the Fc region or moiety may comprise a CH1 domain and a CH2 domain from an IgG4 molecule and a CH3 domain from an IgG1 molecule. For example, the Fc region or moiety may comprise a portion of a CH2 domain from a particular subclass of antibody, e.g., EU positions 292-340 of a CH2 domain. For example, an Fc region or moiety may comprise amino acids a positions 292-340 of CH2 derived from an IgG4 moiety and the remainder of CH2 derived from an IgG1 moiety (alternatively, 292-340 of CH2 may be derived from an IgG1 moiety and the remainder of CH2 derived from an IgG4 moiety).

Moreover, an Fc region or moiety may (additionally or alternatively) for example comprise a chimeric hinge region. For example, the chimeric hinge may be derived, e.g. in part, from an IgG1, IgG2, or IgG4 molecule (e.g., an upper and lower middle hinge sequence) and, in part, from an IgG3 molecule (e.g., an middle hinge sequence). In another example, an Fc region or moiety may comprise a chimeric hinge derived, in part, from an IgG1 molecule and, in part, from an IgG4 molecule. In another example, the chimeric hinge may comprise upper and lower hinge domains from an IgG4 molecule and a middle hinge domain from an IgG1 molecule. Such a chimeric hinge may be made, for example, by introducing a proline substitution (Ser228Pro) at EU position 228 in the middle hinge domain of an IgG4 hinge region. In other embodiments, the chimeric hinge can comprise amino acids at EU positions 233-236 are from an IgG2 antibody and/or the Ser228Pro mutation, wherein the remaining amino acids of the hinge are from an IgG4 antibody (e.g., a chimeric hinge of the sequence ESKYGPPCPPCPAPPVAGP). Further chimeric hinges, which may be used in the Fc moiety of the antibody according to the present invention are described in US 2005/0163783 A1.

In some embodiments, the Fc moiety, or the Fc region, comprises or consists of an amino acid sequence derived from a human immunoglobulin sequence (e.g., from an Fc region or Fc moiety from a human IgG molecule). However, polypeptides may comprise one or more amino acids from another mammalian species. For example, a primate Fc moiety or a primate binding site may be included in the subject polypeptides. Alternatively, one or more murine amino acids may be present in the Fc moiety or in the Fc region.

In some embodiments, the antibody according to the present invention comprises, in particular in addition to an Fc moiety as described above, other parts derived from a constant region, in particular from a constant region of IgG, such as a constant region of (human) IgG1. The antibody according to the present invention may comprise, in particular in addition to an Fc moiety as described above, all other parts of the constant regions, in particular all other parts of the constant regions of IgG (such as (human) IgG1).

Example sequences of constant regions are the amino acid sequences according to SEQ ID NOs: 30-32. For example, the amino acid sequence of IgG1 CH1-CH2-CH3 is according to SEQ ID NO: 30 or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity. In some embodiments, the amino acid sequence of IgG1 CH1-CH2-CH3 may be according to SEQ ID NO: 103 or a sequence variant thereof (including, for example, 1, 2, 3, 4, 5, 6, 7, 8, 9, 10 or more mutations) having at least 70%, at least 75%, at least 80%, at least 85%, at least 90%, at least 95% or at least 99% sequence identity, wherein the mutation(s) M428L and/or N434S may be maintained.

As outlined above, an antibody according to the present invention may comprise a (complete) Fc region derived from human IgG1. In some embodiments, the antibody according to the present invention comprises, in particular in addition to a (complete) Fc region derived from human IgG1 also all other parts of the constant regions of IgG, such as all other parts of the constant regions of (human) IgG1.

In some embodiments, the antibody according to the present invention comprises a (complete) Fc moiety/Fc region, wherein the interaction/binding with FcR is not compromised. In general, binding of the antibody to an Fc receptor may be assessed by various methods known to the skilled person, such as ELISA (Hessell A J, Hangartner L, Hunter M, Havenith C E G, Beurskens F J, Bakker J M, Lanigan C M S, Landucci G, Forthal D N, Parren PWHI, et al.: Fc receptor but not complement binding is important in antibody protection against HIV. Nature 2007, 449:101-104; Grevys A, Bern M, Foss S, Bratlie D B, Moen A, Gunnarsen K S, Aase A, Michaelsen T E, Sandlie I, Andersen J T: Fc Engineering of Human IgG1 for Altered Binding to the Neonatal Fc Receptor Affects Fc Effector Functions. 2015, 194:5497-5508) or flow-cytometry (Perez L G, Costa M R, Todd C A, Haynes B F, Montefiori D C: Utilization of immunoglobulin G Fc receptors by human immunodeficiency virus type 1: a specific role for antibodies against the membrane-proximal external region of gp41. J Virol 2009, 83:7397-7410; Piccoli L, Campo I, Fregni C S, Rodriguez B M F, Minola A, Sallusto F, Luisetti M, Corti D, Lanzavecchia A: Neutralization and clearance of GM-CSF by autoantibodies in pulmonary alveolar proteinosis. Nat Commun 2015, 6:1-9).

In general, the antibody according to the present invention may be glycosylated. N-linked glycans attached to the CH2 domain of a heavy chain, for instance, can influence C1q and FcR binding, with glycosylated antibodies having lower affinity for these receptors. Accordingly, the CH2 domain of the Fc moiety of the antibody according to the present invention may comprise one or more mutations, in which a glycosylated residue is substituted by a non-glycosylated residue. For example, the antibody's glycans do not lead to a human immunogenic response after administration.

Furthermore, the antibody according to the present invention can be modified by introducing (random) amino acid mutations into particular region of the CH2 or CH3 domain of the heavy chain in order to alter their binding affinity for FcR and/or their serum half-life in comparison to unmodified antibodies. Examples of such modifications include, but are not limited to, substitutions of at least one amino acid from the heavy chain constant region selected from the group consisting of amino acid residues 250, 314, and 428. Further examples of such Fc modifications are described in Saxena A, Wu D. Advances in Therapeutic Fc Engineering-Modulation of IgG-Associated Effector Functions and Serum Half-life. Front Immunol. 2016; 7:580, which is incorporated herein by reference. In some embodiments, the antibody may comprise the “YTE” mutations (M252Y/S254T/T256E; EU numbering). In some embodiments, the antibody may comprise the mutations M428L and/or N434S in the heavy chain constant region (EU numbering). For example, the antibody may comprise a heavy chain constant region comprising an amino acid sequence as set forth in SEQ ID NO: 103; or an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 103, wherein the mutations M428L and N434S are maintained.

In some embodiments, the antibody may comprise a heavy chain comprising an amino acid sequence as set forth in SEQ ID NO: 100; or an amino acid sequence having at least 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or more identity to SEQ ID NO: 100, wherein the heavy chain CDR1, CDR2, and CDR3 sequences as set forth in SEQ ID NO: 1, SEQ ID NO: 2, and SEQ ID NO: 12, respectively, and the mutations M428L and N434S are maintained.

In some embodiments, the antibody may comprise a heavy chain comprising an amino acid sequence as set forth in SEQ ID NO: 102; or an amino acid sequence having at least 70%, 71%, 72%, 73%, 74%, 75%, 76%, 77%, 78%, 79%, 80%, 8M), 82%, 83%, 84%, 85%, 86%, 87%, 88%, 89%, 90%, 91%, 92%, 93%, 94%, 95%, 96%, 97%, 98%, 99% or more identity to SEQ ID NO: 102, wherein the heavy chain CDR1, CDR2, and CDR3 sequences as set forth in SEQ ID NO: 17, SEQ ID NO: 18, and SEQ ID NO: 19, respectively, and the mutations M428L and N434S are maintained.

Antibodies of the invention also include hybrid antibody molecules that comprise the six CDRs from an antibody of the invention as defined above and one or more CDRs from another antibody to an antigen. For example, the antibody may be bispecific.

Variant antibodies are also included within the scope of the invention. Thus, variants of the sequences recited in the application are also included within the scope of the invention. Such variants include natural variants generated by somatic mutation in vivo during the immune response or in vitro upon culture of immortalized B cell clones. Alternatively, variants may arise due to the degeneracy of the genetic code or may be produced due to errors in transcription or translation.

Antibodies of the invention may be provided in purified form. Typically, the antibody will be present in a composition that is substantially free of other polypeptides e.g., where less than 90% (by weight), usually less than 60% and more usually less than 50% of the composition is made up of other polypeptides.

Antibodies of the invention may be immunogenic in non-human (or heterologous) hosts e.g., in mice. In particular, the antibodies may have an idiotope that is immunogenic in non-human hosts, but not in a human host. In particular, antibodies of the invention for human use include those that cannot be easily isolated from hosts such as mice, goats, rabbits, rats, non-primate mammals, etc. and cannot generally be obtained by humanization or from xeno-mice.

Nucleic Acids

In another aspect, the invention also provides a nucleic acid molecule comprising a polynucleotide encoding the antibody according to the present invention, or an antigen-binding fragment thereof, as described above.

In some embodiments, the polynucleotide encoding the antibody, or an antigen-binding fragment thereof, may be codon-optimized.

The nucleic acid molecule may comprise a nucleic acid sequence as set forth in any one of SEQ ID NOs 36-39 or 106; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

The present invention also provides a nucleic acid molecule comprising a polynucleotide according to any one of SEQ ID NOs 40-99. Moreover, the present invention also provides a nucleic acid molecule comprising a polynucleotide according to any one of SEQ ID NOs 40-99 or 106.

In some embodiments, the nucleic acid molecule comprises
(i) a polynucleotide according to any one of SEQ ID NOs 40-45 and a polynucleotide according to any one of SEQ ID NOs 46-51; or
(ii) a polynucleotide according to any one of SEQ ID NOs 64-66, a polynucleotide according to any one of SEQ ID NOs 67-69, a polynucleotide according to any one of SEQ ID NOs 70-72, a polynucleotide according to any one of SEQ ID NOs 73-75, a polynucleotide according to any one of SEQ ID NOs 76-78, and a polynucleotide according to any one of SEQ ID NOs 79-81.

In certain embodiments, the nucleic acid molecule comprises
(i) a polynucleotide according to any one of SEQ ID NOs 36, 37, 40-45 and 106, and a polynucleotide according to any one of SEQ ID NOs 38, 46-51; or
(ii) a polynucleotide according to any one of SEQ ID NOs 64-66, a polynucleotide according to any one of SEQ ID NOs 67-69, a polynucleotide according to any one of SEQ ID NOs 70-72, a polynucleotide according to any one of SEQ ID NOs 73-75, a polynucleotide according to any one of SEQ ID NOs 76-78, and a polynucleotide according to any one of SEQ ID NOs 79-81.

In some embodiments, the nucleic acid molecule comprises
(i) a polynucleotide according to any one of SEQ ID NOs 52-57 and a polynucleotide according to any one of SEQ ID NOs 58-63; or
(ii) a polynucleotide according to any one of SEQ ID NOs 82-84, a polynucleotide according to any one of SEQ ID NOs 85-87, a polynucleotide according to any one of SEQ ID NOs 88-90, a polynucleotide according to any one of SEQ ID NOs 91-93, a polynucleotide according to any one of SEQ ID NOs 94-96, and a polynucleotide according to any one of SEQ ID NOs 97-99.

In certain embodiments, the nucleic acid molecule comprises
(i) a polynucleotide according to any one of SEQ ID NOs 52-57 and a polynucleotide according to any one of SEQ ID NOs 39, 58-63; or
(ii) a polynucleotide according to any one of SEQ ID NOs 82-84, a polynucleotide according to any one of SEQ ID NOs 85-87, a polynucleotide according to any one of SEQ ID NOs 88-90, a polynucleotide according to any one of SEQ ID NOs 91-93, a polynucleotide according to any one of SEQ ID NOs 94-96, and a polynucleotide according to any one of SEQ ID NOs 97-99.

The one or more polynucleotide(s) may encode an antibody, in particular or the two variable regions thereof (as described in option (i)) or the six CDRs thereof (as described in option (ii)).

In certain embodiments, the one or more polynucleotide(s) may be selected such that they encode together (i) the six CDRs, (ii) the variable regions VH and VL; or the light and heavy chain of any one of the exemplified antibodies MGU10v1, MGU10v2, MGU10v3, MGU10v4, MGU10v5, MGU10v6, MGU10v7, MGU10v8, MGU10v9, or MGH2v1.

Examples of nucleic acid molecules and/or polynucleotides include, e.g., a recombinant polynucleotide, a vector, an oligonucleotide, an RNA molecule such as an rRNA, an mRNA, an miRNA, an siRNA, or a tRNA, or a DNA molecule such as a cDNA. Nucleic acids may encode the light chain and/or the heavy chain of an antibody. In other words, the light chain and the heavy chain of the antibody may be encoded by the same nucleic acid molecule (e.g., in bicistronic manner). Alternatively, the light chain and the heavy chain of the antibody may be encoded by distinct nucleic acid molecules.

Due to the redundancy of the genetic code, the present invention also comprises sequence variants of nucleic acid sequences, which encode the same amino acid sequences. The polynucleotide encoding the antibody (or the complete nucleic acid molecule) may be optimized for expression of the antibody. For example, codon optimization of the nucleotide sequence may be used to improve the efficiency of translation in expression systems for the production of the antibody. Moreover, the nucleic acid molecule may comprise heterologous elements (i.e., elements, which in nature do not occur on the same nucleic acid molecule as the coding sequence for the (heavy or light chain of) an antibody. For example, a nucleic acid molecule may comprise a heterologous promotor, a heterologous enhancer, a heterologous UTR (e.g., for optimal translation/expression), a heterologous Poly-A-tail, and the like.

A nucleic acid molecule is a molecule comprising nucleic acid components. The term nucleic acid molecule usually refers to DNA or RNA molecules. It may be used synonymous with the term “polynucleotide”, i.e. the nucleic acid molecule may consist of a polynucleotide encoding the antibody. Alternatively, the nucleic acid molecule may also comprise further elements in addition to the polynucleotide encoding the antibody. Typically, a nucleic acid molecule is a polymer comprising or consisting of nucleotide monomers which are covalently linked to each other by phosphodiester-bonds of a sugar/phosphate-backbone. The term “nucleic acid molecule” also encompasses modified nucleic acid molecules, such as base-modified, sugar-modified or backbone-modified etc. DNA or RNA molecules.

In general, the nucleic acid molecule may be manipulated to insert, delete or alter certain nucleic acid sequences. Changes from such manipulation include, but are not limited to, changes to introduce restriction sites, to amend codon usage, to add or optimize transcription and/or translation regulatory sequences, etc. It is also possible to change the nucleic acid to alter the encoded amino acids. For example, it may be useful to introduce one or more (e.g., 1, 2, 3, 4, 5, 6, 7, 8, 9, 10, etc.) amino acid substitutions, deletions and/or insertions into the antibody's amino acid sequence. Such point mutations can modify effector functions, antigen-binding affinity, post-translational modifications, immunogenicity, etc., can introduce amino acids for the attachment of covalent groups (e.g., labels) or can introduce tags (e.g., for purification purposes). Alternatively, a mutation in a nucleic acid sequence may be “silent”, i.e. not reflected in the amino acid sequence due to the redundancy of the genetic code. In general, mutations can be introduced in specific sites or can be introduced at random, followed by selection (e.g., molecular evolution). For instance, one or more nucleic acids encoding any of the light or heavy chains of an (exemplary) antibody can be randomly or directionally mutated to introduce different properties in the encoded amino acids. Such changes can be the result of an iterative process wherein initial changes are retained and new changes at other nucleotide positions are introduced. Further, changes achieved in independent steps may be combined.

In some embodiments, the polynucleotide encoding the antibody, or an antigen-binding fragment thereof, (or the (complete) nucleic acid molecule) may be codon-optimized. The skilled artisan is aware of various tools for codon optimization, such as those described in: Ju Xin Chin, Bevan Kai-Sheng Chung, Dong-Yup Lee, Codon Optimization OnLine (COOL): a web-based multi-objective optimization platform for synthetic gene design, Bioinformatics, Volume 30, Issue 15, 1 Aug. 2014, Pages 2210-2212; or in: Grote A, Hiller K, Scheer M, Munch R, Nortemann B, Hempel D C, Jahn D, JCat: a novel tool to adapt codon usage of a target gene to its potential expression host. Nucleic Acids Res. 2005 Jul. 1; 33(Web Server issue):W526-31; or, for example, Genscript's OptimumGene™ algorithm (as described in US 2011/0081708 A1).

For example, the nucleic acid molecule of the invention may comprise a nucleic acid sequence as set forth in any one of SEQ ID NOs 36-39; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

The present invention also provides a combination of a first and a second nucleic acid molecule, wherein the first nucleic acid molecule comprises a polynucleotide encoding the heavy chain of the antibody, or an antigen-binding fragment thereof, of the present invention; and the second nucleic acid molecule comprises a polynucleotide encoding the corresponding light chain of the same antibody, or the same antigen-binding fragment thereof. The above description regarding the (general) features of the nucleic acid molecule of the invention applies accordingly to the first and second nucleic acid molecule of the combination. Accordingly, one or both of the polynucleotides encoding the heavy and/or light chain(s) of the antibody, or an antigen-binding fragment thereof, is/are codon-optimized. For example, the combination may comprise a nucleic acid sequence as set forth in any one of SEQ ID NOs 36-39 or 106; or a sequence variant thereof having at least 70%, at least 75%, at least 80%, at least 85%, at least 88%, at least 90%, at least 92%, at least 95%, at least 96%, at least 97%, at least 98% or at least 99% sequence identity.

The present invention also provides a combination of a first and a second nucleic acid molecule, wherein
the first nucleic acid molecule comprises a polynucleotide encoding the heavy chain of an antibody, or an antigen-binding fragment thereof, the polynucleotide comprising: (a) a nucleotide sequence according to any one of SEQ ID NOs 40-45; or (b) a nucleotide sequence according to any one of SEQ ID NOs 64-66, a nucleotide sequence according to any one of SEQ ID NOs 67-69, and a nucleotide sequence according to any one of SEQ ID NOs 70-72; and
the second nucleic acid molecule comprises a polynucleotide encoding the light chain of an antibody, or an antigen-binding fragment thereof, the polynucleotide comprising: (c) a nucleotide sequence according to any one of SEQ ID NOs 46-51; or (d) a nucleotide sequence according to any one of SEQ ID NOs 73-75, a nucleotide sequence according to any one of SEQ ID NOs 76-78, and a nucleotide sequence according to any one of SEQ ID NOs 79-81.

Again, the above description regarding the (general) features of the nucleic acid molecule of the invention applies accordingly to the first and second nucleic acid molecule of the combination.

The present invention also provides a combination of a first and a second nucleic acid molecule, wherein
the first nucleic acid molecule comprises a polynucleotide encoding the heavy chain of an antibody, or an antigen-binding fragment thereof, the polynucleotide comprising: (a) a nucleotide sequence according to any one of SEQ ID NOs 36, 37, 40-45 and 106; or (b) a nucleotide sequence according to any one of SEQ ID NOs 64-66, a nucleotide sequence according to any one of SEQ ID NOs 67-69, and a nucleotide sequence according to any one of SEQ ID NOs 70-72; and
the second nucleic acid molecule comprises a polynucleotide encoding the light chain of an antibody, or an antigen-binding fragment thereof, the polynucleotide comprising: (c) a nucleotide sequence according to any one of SEQ ID NOs 38, 46-51; or (d) a nucleotide sequence according to any one of SEQ ID NOs 73-75, a nucleotide sequence according to any one of SEQ ID NOs 76-78, and a nucleotide sequence according to any one of SEQ ID NOs 79-81.

The present invention also provides a combination of a first and a second nucleic acid molecule, wherein
the first nucleic acid molecule comprises a polynucleotide encoding the heavy chain of an antibody, or an antigen-binding fragment thereof, the polynucleotide comprising: (a) a nucleotide sequence according to any one of SEQ ID NOs 52-57; or (b) a nucleotide sequence according to any one of SEQ ID NOs 82-84, a nucleotide sequence according to any one of SEQ ID NOs 85-87, and a nucleotide sequence according to any one of SEQ ID NOs 88-90; and
the second nucleic acid molecule comprises a polynucleotide encoding the light chain of an antibody, or an antigen-binding fragment thereof, the polynucleotide comprising: (c) a nucleotide sequence according to any one of SEQ ID NOs 58-63; or (d) a nucleotide sequence according to any one of SEQ ID NOs 91-93, a nucleotide sequence according to any one of SEQ ID NOs 94-96, and a nucleotide sequence according to any one of SEQ ID NOs 97-99.

The present invention also provides a combination of a first and a second nucleic acid molecule, wherein
the first nucleic acid molecule comprises a polynucleotide encoding the heavy chain of an antibody, or an antigen-binding fragment thereof, the polynucleotide comprising: (a) a nucleotide sequence according to any one of SEQ ID NOs 52-57; or (b) a nucleotide sequence according to any one of SEQ ID NOs 82-84, a nucleotide sequence according to any one of SEQ ID NOs 85-87, and a nucleotide sequence according to any one of SEQ ID NOs 88-90; and
the second nucleic acid molecule comprises a polynucleotide encoding the light chain of an antibody, or an antigen-binding fragment thereof, the polynucleotide comprising: (c) a nucleotide sequence according to any one of SEQ ID NOs 39, 58-63; or (d) a nucleotide sequence according to any one of SEQ ID NOs 91-93, a nucleotide sequence according to any one of SEQ ID NOs 94-96, and a nucleotide sequence according to any one of SEQ ID NOs 97-99.

In particular, the first and the second nucleic acid molecules may be selected such that they encode together (i) the six CDRs, (ii) the variable regions VH and VL; or the light and heavy chain of any one of the exemplified antibodies MGU10v1, MGU10v2, MGU10v3, MGU10v4, MGU10v5, MGU10v6, MGU10v7, MGU10v8, MGU10v9, or MGH2v1.

Again, the above description regarding the (general) features of the nucleic acid molecule of the invention applies accordingly to the first and second nucleic acid molecule of the combination.

The nucleic acid sequences of SEQ ID NOs 40-99 and 106 are codon-optimized for antibody expression.

Vector

Further included within the scope of the invention are vectors, for example, expression vectors, comprising a nucleic acid molecule according to the present invention. Usually, a vector comprises a nucleic acid molecule as described above.

The present invention also provides a combination of a first and a second vector, wherein the first vector comprises a first nucleic acid molecule as described above (for the combination of nucleic acid molecules) and the second vector comprises a second nucleic acid molecule as described above (for the combination of nucleic acid molecules), in particular wherein the first and the second nucleic acid molecules are selected from the same (embodiment of) combination of nucleic acid molecules as described above. More specifically, the first and the second nucleic acid molecules may be selected such that they encode together (i) the six CDRs, (ii) the variable regions VH and VL; or the light and heavy chain of any one of the exemplified antibodies MGU10v1, MGU10v2, MGU10v3, MGU10v4, MGU10v5, MGU10v6, MGU10v7, MGU10v8, MGU10v9, or MGH2v1.

A vector is usually a recombinant nucleic acid molecule, i.e. a nucleic acid molecule which does not occur in nature. Accordingly, the vector may comprise heterologous elements (i.e., sequence elements of different origin in nature). For example, the vector may comprise a multi cloning site, a heterologous promotor, a heterologous enhancer, a heterologous selection marker (to identify cells comprising said vector in comparison to cells not comprising said vector) and the like. A vector in the context of the present invention is suitable for incorporating or harboring a desired nucleic acid sequence. Such vectors may be storage vectors, expression vectors, cloning vectors, transfer vectors etc. A storage vector is a vector which allows the convenient storage of a nucleic acid molecule. Thus, the vector may comprise a sequence corresponding, e.g., to a (heavy and/or light chain of a) desired antibody according to the present invention. An expression vector may be used for production of expression products such as RNA, e.g. mRNA, or peptides, polypeptides or proteins. For example, an expression vector may comprise sequences needed for transcription of a sequence stretch of the vector, such as a (heterologous) promoter sequence. A cloning vector is typically a vector that contains a cloning site, which may be used to incorporate nucleic acid sequences into the vector. A cloning vector may be, e.g., a plasmid vector or a bacteriophage vector. A transfer vector may be a vector which is suitable for transferring nucleic acid molecules into cells or organisms, for example, viral vectors. A vector in the context of the present invention may be, e.g., an RNA vector or a DNA vector. For example, a vector in the sense of the present application comprises a cloning site, a selection marker, such as an antibiotic resistance factor, and a sequence suitable for multiplication of the vector, such as an origin of replication. A vector in the context of the present application may be a plasmid vector.

Cells

In a further aspect, the present invention also provides cell expressing the antibody according to the present invention; and/or comprising the vector (or the combination of vectors) according the present invention.

Examples of such cells include but are not limited to, eukaryotic cells, e.g., yeast cells, animal cells or plant cells. Other examples of such cells include but are not limited, to prokaryotic cells, e.g. E. coli. In some embodiments, the cells are mammalian cells, such as a mammalian cell line. Examples include human cells, CHO cells, HEK293T cells, PER.C6 cells, NS0 cells, human liver cells, myeloma cells or hybridoma cells.

The cell may be transfected with a vector according to the present invention, for example with an expression vector. The term “transfection” refers to the introduction of nucleic acid molecules, such as DNA or RNA (e.g. mRNA) molecules, into cells, e.g. into eukaryotic or prokaryotic cells. In the context of the present invention, the term “transfection” encompasses any method known to the skilled person for introducing nucleic acid molecules into cells, such as into mammalian cells. Such methods encompass, for example, electroporation, lipofection, e.g. based on cationic lipids and/or liposomes, calcium phosphate precipitation, nanoparticle based transfection, virus based transfection, or transfection based on cationic polymers, such as DEAE-dextran or polyethylenimine etc. In some embodiments, the introduction is non-viral.

Moreover, the cells of the present invention may be transfected stably or transiently with the vector according to the present invention, e.g. for expressing the antibody according to the present invention. In some embodiments, the cells are stably transfected with the vector according to the present invention encoding the antibody according to the present invention. In other embodiments, the cells are transiently transfected with the vector according to the present invention encoding the antibody according to the present invention.

Accordingly, the present invention also provides a recombinant host cell, which heterologously expresses the antibody of the invention or the antigen-binding fragment thereof. For example, the cell may be of another species than the antibody (e.g., CHO cells expressing human antibodies). In some embodiments, the cell type of the cell does not express (such) antibodies in nature. Moreover, the host cell may impart a post-translational modification (PTM; e.g., glycosylation) on the antibody that is not present in their native state. Such a PTM may result in a functional difference (e.g., reduced immunogenicity). Accordingly, the antibody of the invention, or the antigen-binding fragment thereof, may have a post-translational modification, which is distinct from the naturally produced antibody (e.g., an antibody of an immune response in a human).

Production of Antibodies

Antibodies according to the invention can be made by any method known in the art. For example, the general methodology for making monoclonal antibodies using hybridoma technology is well known (Kohler, G. and Milstein, C, 1975; Kozbar et al. 1983). In some embodiments, the alternative EBV immortalization method described in WO2004/076677 is used.

In some embodiments, the method as described in WO 2004/076677, which is incorporated herein by reference, is used. In this method B cells producing the antibody of the invention are transformed with EBV and a polyclonal B cell activator. Additional stimulants of cellular growth and differentiation may optionally be added during the transformation step to further enhance the efficiency. These stimulants may be cytokines such as IL-2 and IL-15. In one aspect, IL-2 is added during the immortalization step to further improve the efficiency of immortalization, but its use is not essential. The immortalized B cells produced using these methods can then be cultured using methods known in the art and antibodies isolated therefrom.

Another exemplified method is described in WO 2010/046775. In this method plasma cells are cultured in limited numbers, or as single plasma cells in microwell culture plates. Antibodies can be isolated from the plasma cell cultures. Further, from the plasma cell cultures, RNA can be extracted and PCR can be performed using methods known in the art.

The VH and VL regions of the antibodies can be amplified by RT-PCR (reverse transcriptase PCR), sequenced and cloned into an expression vector that is then transfected into HEK293T cells or other host cells. The cloning of nucleic acid in expression vectors, the transfection of host cells, the culture of the transfected host cells and the isolation of the produced antibody can be done using any methods known to one of skill in the art.

The antibodies may be further purified, if desired, using filtration, centrifugation and various chromatographic methods such as HPLC or affinity chromatography. Techniques for purification of antibodies, e.g., monoclonal antibodies, including techniques for producing pharmaceutical-grade antibodies, are well known in the art.

Standard techniques of molecular biology may be used to prepare DNA sequences encoding the antibodies of the present invention. Desired DNA sequences may be synthesized completely or in part using oligonucleotide synthesis techniques. Site-directed mutagenesis and polymerase chain reaction (PCR) techniques may be used as appropriate.

Any suitable host cell/vector system may be used for expression of the DNA sequences encoding the antibody molecules of the present invention. Eukaryotic, e.g., mammalian, host cell expression systems may be used for production of antibody molecules, such as complete antibody molecules. Suitable mammalian host cells include, but are not limited to, CHO, HEK293T, PER.C6, NS0, myeloma or hybridoma cells. Also, prokaryotic, e.g. bacterial host cell expression systems may be used for the production of antibody molecules, such as complete antibody molecules. Suitable bacterial host cells include, but are not limited to, E. coli cells.

The present invention also provides a process for the production of an antibody molecule according to the present invention comprising culturing a (heterologous) host cell comprising a vector encoding a nucleic acid of the present invention under conditions suitable for expression of protein from DNA encoding the antibody molecule of the present invention, and isolating the antibody molecule.

For production of the antibody comprising both heavy and light chains, a cell line may be transfected with two vectors, a first vector encoding a light chain polypeptide and a second vector encoding a heavy chain polypeptide. Alternatively, a single vector may be used, the vector including sequences encoding light chain and heavy chain polypeptides.

Antibodies according to the invention may be produced by (i) expressing a nucleic acid sequence according to the invention in a host cell, e.g. by use of a vector according to the present invention, and (ii) isolating the expressed antibody product. Additionally, the method may include (iii) purifying the isolated antibody. Transformed B cells and cultured plasma cells may be screened for those producing antibodies of the desired specificity or function.

The screening step may be carried out by any immunoassay, e.g., ELISA, by staining of tissues or cells (including transfected cells), by neutralization assay or by one of a number of other methods known in the art for identifying desired specificity or function. The assay may select on the basis of simple recognition of one or more antigens, or may select on the additional basis of a desired function e.g., to select neutralizing antibodies rather than just antigen-binding antibodies, to select antibodies that can change characteristics of targeted cells, such as their signaling cascades, their shape, their growth rate, their capability of influencing other cells, their response to the influence by other cells or by other reagents or by a change in conditions, their differentiation status, etc.

Individual transformed B cell clones may then be produced from the positive transformed B cell culture. The cloning step for separating individual clones from the mixture of positive cells may be carried out using limiting dilution, micromanipulation, single cell deposition by cell sorting or another method known in the art.

Nucleic acid from the cultured plasma cells can be isolated, cloned and expressed in HEK293T cells or other known host cells using methods known in the art.

The immortalized B cell clones or the transfected host-cells of the invention can be used in various ways e.g., as a source of monoclonal antibodies, as a source of nucleic acid (DNA or mRNA) encoding a monoclonal antibody of interest, for research, etc.

The invention also provides a composition comprising immortalized B memory cells or transfected host cells that produce antibodies according to the present invention.

The immortalized B cell clone or the cultured plasma cells of the invention may also be used as a source of nucleic acid for the cloning of antibody genes for subsequent recombinant expression. Expression from recombinant sources may be more common for pharmaceutical purposes than expression from B cells or hybridomas e.g., for reasons of stability, reproducibility, culture ease, etc.

Thus the invention also provides a method for preparing a recombinant cell, comprising the steps of: (i) obtaining one or more nucleic acids (e.g., heavy and/or light chain mRNAs) from the B cell clone or the cultured plasma cells that encodes the antibody of interest; (ii) inserting the nucleic acid into an expression vector and (iii) transfecting the vector into a (heterologous) host cell in order to permit expression of the antibody of interest in that host cell.

Similarly, the invention also provides a method for preparing a recombinant cell, comprising the steps of: (i) sequencing nucleic acid(s) from the B cell clone or the cultured plasma cells that encodes the antibody of interest; and (ii) using the sequence information from step (i) to prepare nucleic acid(s) for insertion into a host cell in order to permit expression of the antibody of interest in that host cell. The nucleic acid may, but need not, be manipulated between steps (i) and (ii) to introduce restriction sites, to change codon usage, and/or to optimize transcription and/or translation regulatory sequences.

Furthermore, the invention also provides a method of preparing a transfected host cell, comprising the step of transfecting a host cell with one or more nucleic acids that encode an antibody of interest, wherein the nucleic acids are nucleic acids that were derived from an immortalized B cell clone or a cultured plasma cell of the invention. Thus the procedures for first preparing the nucleic acid(s) and then using it to transfect a host cell can be performed at different times by different people in different places (e.g., in different countries).

These recombinant cells of the invention can then be used for expression and culture purposes. They are particularly useful for expression of antibodies for large-scale pharmaceutical production. They can also be used as the active ingredient of a pharmaceutical composition. Any suitable culture technique can be used, including but not limited to static culture, roller bottle culture, ascites fluid, hollow-fiber type bioreactor cartridge, modular minifermenter, stirred tank, microcarrier culture, ceramic core perfusion, etc.

Methods for obtaining and sequencing immunoglobulin genes from B cells or plasma cells are well known in the art (e.g., see Chapter 4 of Kuby Immunology, 4th edition, 2000).

The transfected host cell may be a eukaryotic cell, including yeast and animal cells, particularly mammalian cells (e.g., CHO cells, NS0 cells, human cells such as PER.C6 or HKB-11 cells, myeloma cells, or a human liver cell), as well as plant cells. In some embodiments, the transfected host cell is a mammalian cell, such as a human cell. In some embodiments, expression hosts can glycosylate the antibody of the invention, particularly with carbohydrate structures that are not themselves immunogenic in humans. In some embodiments the transfected host cell may be able to grow in serum-free media. In further embodiments the transfected host cell may be able to grow in culture without the presence of animal-derived products. The transfected host cell may also be cultured to give a cell line.

The invention also provides a method for preparing one or more nucleic acid molecules (e.g., heavy and light chain genes) that encode an antibody of interest, comprising the steps of: (i) preparing an immortalized B cell clone or culturing plasma cells according to the invention; (ii) obtaining from the B cell clone or the cultured plasma cells nucleic acid that encodes the antibody of interest. Further, the invention provides a method for obtaining a nucleic acid sequence that encodes an antibody of interest, comprising the steps of: (i) preparing an immortalized B cell clone or culturing plasma cells according to the invention; (ii) sequencing nucleic acid from the B cell clone or the cultured plasma cells that encodes the antibody of interest.

The invention further provides a method of preparing nucleic acid molecule(s) that encode an antibody of interest, comprising the step of obtaining the nucleic acid that was obtained from a transformed B cell clone or cultured plasma cells of the invention. Thus the procedures for first obtaining the B cell clone or the cultured plasma cell, and then obtaining nucleic acid(s) from the B cell clone or the cultured plasma cells can be performed at different times by different people in different places (e.g., in different countries).

The invention also comprises a method for preparing an antibody (e.g., for pharmaceutical use) according to the present invention, comprising the steps of: (i) obtaining and/or sequencing one or more nucleic acids (e.g., heavy and light chain genes) from the selected B cell clone or the cultured plasma cells expressing the antibody of interest; (ii) inserting the nucleic acid(s) into or using the nucleic acid(s) sequence(s) to prepare an expression vector; (iii) transfecting a host cell that can express the antibody of interest; (iv) culturing or sub-culturing the transfected host cells under conditions where the antibody of interest is expressed; and, optionally, (v) purifying the antibody of interest.

The invention also provides a method of preparing the antibody of interest comprising the steps of: culturing or sub-culturing a transfected host cell population, e.g. a stably transfected host cell population, under conditions where the antibody of interest is expressed and, optionally, purifying the antibody of interest, wherein said transfected host cell population has been prepared by (i) providing nucleic acid(s) encoding a selected antibody of interest that is produced by a B cell clone or cultured plasma cells prepared as described above, (ii) inserting the nucleic acid(s) into an expression vector, (iii) transfecting the vector in a host cell that can express the antibody of interest, and (iv) culturing or sub-culturing the transfected host cell comprising the inserted nucleic acids to produce the antibody of interest. Thus the procedures for first preparing the recombinant host cell and then culturing it to express antibody can be performed at very different times by different people in different places (e.g., in different countries).

Pharmaceutical Composition

The present invention also provides a pharmaceutical composition comprising one or more of:
(i) the antibody of to the present invention, or an antigen-binding fragment thereof;
(ii) the nucleic acid or a combination of nucleic acids according to the present invention;
(iii) the vector or a combination of vectors according to the present invention; and/or
(iv) the cell expressing the antibody according to the present invention or comprising the vector according to the present invention

and, optionally, a pharmaceutically acceptable diluent or carrier.

In other words, the present invention also provides a pharmaceutical composition comprising the antibody according to the present invention, the nucleic acid according to the present invention, the vector according to the present invention and/or the cell according to the present invention.

The pharmaceutical composition may optionally also contain a pharmaceutically acceptable carrier, diluent and/or excipient. Although the carrier or excipient may facilitate administration, it should not itself induce the production of antibodies harmful to the individual receiving the composition. Nor should it be toxic. Suitable carriers may be large, slowly metabolized macromolecules such as proteins, polypeptides, liposomes, polysaccharides, polylactic acids, polyglycolic acids, polymeric amino acids, amino acid copolymers and inactive virus particles. In some embodiments, the pharmaceutically acceptable carrier, diluent and/or excipient in the pharmaceutical composition according to the present invention is not an active component in respect to P. falciparum infection and/or malaria.

Pharmaceutically acceptable salts can be used, for example mineral acid salts, such as hydrochlorides, hydrobromides, phosphates and sulphates, or salts of organic acids, such as acetates, propionates, malonates and benzoates.

Pharmaceutically acceptable carriers in a pharmaceutical composition may additionally contain liquids such as water, saline, glycerol and ethanol. Additionally, auxiliary substances, such as wetting or emulsifying agents or pH buffering substances, may be present in such compositions. Such carriers enable the pharmaceutical compositions to be formulated as tablets, pills, dragees, capsules, liquids, gels, syrups, slurries and suspensions, for ingestion by the subject.

Pharmaceutical compositions of the invention may be prepared in various forms. For example, the compositions may be prepared as injectables, either as liquid solutions or suspensions. Solid forms suitable for solution in, or suspension in, liquid vehicles prior to injection can also be prepared (e.g., a lyophilized composition, similar to Synagis™ and Herceptin®, for reconstitution with sterile water containing a preservative). The composition may be prepared for topical administration e.g., as an ointment, cream or powder. The composition may be prepared for oral administration e.g., as a tablet or capsule, as a spray, or as a syrup (optionally flavored). The composition may be prepared for pulmonary administration e.g., as an inhaler, using a fine powder or a spray. The composition may be prepared as a suppository or pessary. The composition may be prepared for nasal, aural or ocular administration e.g., as drops. The composition may be in kit form, designed such that a combined composition is reconstituted just prior to administration to a subject. For example, a lyophilized antibody may be provided in kit form with sterile water or a sterile buffer.

In some embodiments, the (only) active ingredient in the composition is the antibody according to the present invention. As such, it may be susceptible to degradation in the gastrointestinal tract. Thus, if the composition is to be administered by a route using the gastrointestinal tract, the composition may contain agents which protect the antibody from degradation but which release the antibody once it has been absorbed from the gastrointestinal tract.

A thorough discussion of pharmaceutically acceptable carriers is available in Gennaro (2000) Remington: The Science and Practice of Pharmacy, 20th edition, ISBN: 0683306472.

Pharmaceutical compositions of the invention generally have a pH between 5.5 and 8.5, in some embodiments this may be between 6 and 8, for example about 7. The pH may be maintained by the use of a buffer. The composition may be sterile and/or pyrogen free. The composition may be isotonic with respect to humans. In some embodiments pharmaceutical compositions of the invention are supplied in hermetically-sealed containers.

Within the scope of the invention are compositions present in several forms of administration; the forms include, but are not limited to, those forms suitable for parenteral administration, e.g., by injection or infusion, for example by bolus injection or continuous infusion. Where the product is for injection or infusion, it may take the form of a suspension, solution or emulsion in an oily or aqueous vehicle and it may contain formulatory agents, such as suspending, preservative, stabilizing and/or dispersing agents. Alternatively, the antibody may be in dry form, for reconstitution before use with an appropriate sterile liquid.

A vehicle is typically understood to be a material that is suitable for storing, transporting, and/or administering a compound, such as a pharmaceutically active compound, in particular the antibodies according to the present invention. For example, the vehicle may be a physiologically acceptable liquid, which is suitable for storing, transporting, and/or administering a pharmaceutically active compound, in particular the antibodies according to the present invention. Once formulated, the compositions of the invention can be administered directly to the subject. In some embodiments the compositions are adapted for administration to mammalian, e.g., human subjects.

The pharmaceutical compositions of this invention may be administered by any number of routes including, but not limited to, oral, intravenous, intramuscular, intra-arterial, intramedullary, intraperitoneal, intrathecal, intraventricular, transdermal, transcutaneous, topical, subcutaneous, intranasal, enteral, sublingual, intravaginal or rectal routes. Hyposprays may also be used to administer the pharmaceutical compositions of the invention. Optionally, the pharmaceutical composition may be prepared for oral administration, e.g. as tablets, capsules and the like, for topical administration, or as injectable, e.g. as liquid solutions or suspensions. In some embodiments, the pharmaceutical composition is an injectable. Solid forms suitable for solution in, or suspension in, liquid vehicles prior to injection are also encompassed, for example the pharmaceutical composition may be in lyophilized form.

For injection, e.g. intravenous, cutaneous or subcutaneous injection, or injection at the site of affliction, the active ingredient may be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilizers, buffers, antioxidants and/or other additives may be included, as required. Whether it is an antibody, a peptide, a nucleic acid molecule, or another pharmaceutically useful compound according to the present invention that is to be given to an individual, administration is usually in a “prophylactically effective amount” or a “therapeutically effective amount” (as the case may be), this being sufficient to show benefit to the individual. The actual amount administered, and rate and time-course of administration, will depend on the nature and severity of what is being treated. For injection, the pharmaceutical composition according to the present invention may be provided for example in a pre-filled syringe.

The inventive pharmaceutical composition as defined above may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredient, i.e. the inventive transporter cargo conjugate molecule as defined above, is combined with emulsifying and suspending agents. If desired, certain sweetening, flavoring or coloring agents may also be added.

The inventive pharmaceutical composition may also be administered topically, especially when the target of treatment includes areas or organs readily accessible by topical application, e.g. including accessible epithelial tissue. Suitable topical formulations are readily prepared for each of these areas or organs. For topical applications, the inventive pharmaceutical composition may be formulated in a suitable ointment, containing the inventive pharmaceutical composition, particularly its components as defined above, suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water.

Dosage treatment may be a single dose schedule or a multiple dose schedule. In particular, the pharmaceutical composition may be provided as single-dose product. In some embodiments, the amount of the antibody in the pharmaceutical composition—in particular if provided as single-dose product—does not exceed 200 mg, for example it does not exceed 100 mg or 50 mg.

For a single dose, e.g. a daily, weekly or monthly dose, the amount of the antibody in the pharmaceutical composition according to the present invention, may not exceed 1 g or 500 mg. In some embodiments, for a single dose, the amount of the antibody in the pharmaceutical composition according to the present invention, may not exceed 200 mg, or 100 mg. For example, for a single dose, the amount of the antibody in the pharmaceutical composition according to the present invention, may not exceed 50 mg.

Pharmaceutical compositions typically include an “effective” amount of one or more antibodies of the invention, i.e. an amount that is sufficient to treat, ameliorate, attenuate, reduce or prevent a desired disease or condition, or to exhibit a detectable therapeutic effect. Therapeutic effects also include reduction or attenuation in pathogenic potency or physical symptoms. The precise effective amount for any particular subject will depend upon their size, weight, and health, the nature and extent of the condition, and the therapeutics or combination of therapeutics selected for administration. The effective amount for a given situation is determined by routine experimentation and is within the judgment of a clinician. For purposes of the present invention, an effective dose may generally be from about 0.005 to about 100 mg/kg, for example from about 0.0075 to about 50 mg/kg or from about 0.01 to about 10 mg/kg. In some embodiments, the effective dose will be from about 0.02 to about 5 mg/kg, of the antibody of the present invention (e.g. amount of the antibody in the pharmaceutical composition) in relation to the bodyweight (e.g., in kg) of the individual to which it is administered.

Moreover, the pharmaceutical composition according to the present invention may also comprise an additional active component, which may be a further antibody or a component, which is not an antibody. Accordingly, the pharmaceutical composition according to the present invention may comprise one or more of the additional active components.

The antibody according to the present invention can be present either in the same pharmaceutical composition as the additional active component or, alternatively, the antibody according to the present invention is comprised by a first pharmaceutical composition and the additional active component is comprised by a second pharmaceutical composition different from the first pharmaceutical composition. Accordingly, if more than one additional active component is envisaged, each additional active component and the antibody according to the present invention may be comprised in a different pharmaceutical composition. Such different pharmaceutical compositions may be administered either combined/simultaneously or at separate times or at separate locations (e.g. separate parts of the body).

The antibody according to the present invention and the additional active component may provide an additive therapeutic effect, such as a synergistic therapeutic effect. The term “synergy” is used to describe a combined effect of two or more active agents that is greater than the sum of the individual effects of each respective active agent. Thus, where the combined effect of two or more agents results in “synergistic inhibition” of an activity or process, it is intended that the inhibition of the activity or process is greater than the sum of the inhibitory effects of each respective active agent. The term “synergistic therapeutic effect” refers to a therapeutic effect observed with a combination of two or more therapies wherein the therapeutic effect (as measured by any of a number of parameters) is greater than the sum of the individual therapeutic effects observed with the respective individual therapies.

In some embodiments, a composition of the invention may include antibodies of the invention, wherein the antibodies may make up at least 50% by weight (e.g., 60%, 70%, 75%, 80%, 85%, 90%, 95%, 96%, 97%, 98%, 99% or more) of the total protein in the composition. In the composition of the invention, the antibodies may be in purified form.

The present invention also provides a method of preparing a pharmaceutical composition comprising the steps of: (i) preparing an antibody of the invention; and (ii) admixing the purified antibody with one or more pharmaceutically-acceptable carriers.

In other embodiments, a method of preparing a pharmaceutical composition comprises the step of: admixing an antibody with one or more pharmaceutically-acceptable carriers, wherein the antibody is a monoclonal antibody that was obtained from a transformed B cell or a cultured plasma cell of the invention.

As an alternative to delivering antibodies or B cells for therapeutic purposes, it is possible to deliver nucleic acid (typically DNA) that encodes the monoclonal antibody of interest derived from the B cell or the cultured plasma cells to a subject, such that the nucleic acid can be expressed in the subject in situ to provide a desired therapeutic effect. Suitable gene therapy and nucleic acid delivery vectors are known in the art.

Pharmaceutical compositions may include an antimicrobial, particularly if packaged in a multiple dose format. They may comprise detergent e.g., a Tween (polysorbate), such as Tween 80. Detergents are generally present at low levels e.g., less than 0.01°/o. Compositions may also include sodium salts (e.g., sodium chloride) to give tonicity. For example, a concentration of 10±2 mg/ml NaCl is typical.

Further, pharmaceutical compositions may comprise a sugar alcohol (e.g., mannitol) or a disaccharide (e.g., sucrose or trehalose) e.g., at around 15-30 mg/ml (e.g., 25 mg/ml), particularly if they are to be lyophilized or if they include material which has been reconstituted from lyophilized material. The pH of a composition for lyophilization may be adjusted to between 5 and 8, or between 5.5 and 7, or around 6.1 prior to lyophilization.

The compositions of the invention may also comprise one or more immunoregulatory agents. In some embodiments, one or more of the immunoregulatory agents include(s) an adjuvant.

Medical Treatments and Uses

In a further aspect, the present invention provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention or the pharmaceutical composition according to the present invention in prophylaxis and/or treatment of malaria; or in (ii) diagnosis of malaria. Accordingly, the present invention also provides a method of reducing malaria, or lowering the risk of P. falciparum infection, comprising: administering to a subject in need thereof, a therapeutically effective amount of the antibody, or an antigen-binding fragment thereof, according to the present invention, the nucleic acid molecule (or the combination of nucleic acid molecules) according to the present invention, the vector (or the combination of vectors) according to the present invention, the cell according to the present invention or the pharmaceutical composition according to the present invention. Moreover, the present invention also provides the use of the antibody according to the present invention, or an antigen-binding fragment thereof, the nucleic acid molecule (or the combination of nucleic acid molecules) according to the present invention, the vector (or the combination of vectors) according to the present invention, the cell according to the present invention or the pharmaceutical composition according to the present invention in the manufacture of a medicament for prophylaxis, treatment or attenuation of malaria.

Methods of diagnosis may include contacting an antibody with a sample. Such samples may be isolated from a subject, for example an isolated tissue sample taken from, for example, nasal passages, sinus cavities, salivary glands, lung, liver, pancreas, kidney, ear, eye, placenta, alimentary tract, heart, ovaries, pituitary, adrenals, thyroid, brain, skin or blood, such as plasma or serum. The methods of diagnosis may also include the detection of an antigen/antibody complex, in particular following the contacting of an antibody with a sample. Such a detection step is typically performed at the bench, i.e. without any contact to the human or animal body. Examples of detection methods are well-known to the person skilled in the art and include, e.g., ELISA (enzyme-linked immunosorbent assay).

Prophylaxis of malaria refers in particular to prophylactic settings, wherein the subject was not diagnosed with malaria (either no diagnosis was performed or diagnosis results were negative) and/or the subject does not show symptoms of malaria. In therapeutic settings, in contrast, the subject is typically diagnosed with malaria and/or showing symptoms of malaria. Of note, the terms “treatment” and “therapy”/“therapeutic” of malaria include (complete) cure as well as attenuation/reduction of malaria and/or related symptoms.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows for Example 3 the results of the sporozoite invasion/maturation assay for a control antibody (A) and for antibodies MGU10 (B), MGU10v2 (C) and MGU10v2_LS (D) with five dilutions tested for each antibody.

FIG. 2 shows for Example 4 the results of the sporozoite gliding assay for a control antibody (A) and for antibodies MGU10 (B) and MGU10v2_LS (C) with five dilutions tested for each antibody.

FIG. 3 shows for Example 5 the results of the sporozoite traversal assay for a control antibody (A) and for antibodies MGU10 (B) and MGU10v2_LS (C) with five dilutions tested for each antibody.

FIG. 4 shows for Example 6 the dimerization (Dimers) and aggregation (HMWs) of antibodies MGU10v2_LS (upper panel) and MGU10_LS (lower panel) at 40° C. in two distinct buffers as indicated.

FIG. 5 shows for Example 7 the results of the in vivo protection conferred by MGU10 antibodies in a challenge study; with bioluminescence (A) and percent inhibition (B).

FIG. 6 shows for Example 8 the results of the in vivo protection conferred by MGH2 antibodies in a challenge study; with bioluminescence (A) and percent inhibition (B).

FIG. 7 shows for Example 9 the binding of MGU10v2 and MGU10v8 to peptides NANP (A) and NPDP19 (B).

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1: Design of Variants of Antibodies MGU10 and MGH2

Recently, very potent anti-malaria antibodies were described, which are specific for Plasmodium falciparum circumsporozoite protein (CSP) (Tan J, Sack B K, Oyen D, et al. A public antibody lineage that potently inhibits malaria infection through dual binding to the circumsporozoite protein. Nat Med. 2018; 24(4):401-407. doi:10.1038/nm.4513). The most potent dual-specific antibodies described in this study include antibodies “MGU10” and “MGH2”.

Based thereon, the present inventors designed the following variants of MGU10 (SEQ ID NOs: 1-8) and MGH2 (SEQ ID NOs: 17-27), which exhibit amino acid mutations in the heavy and/or light chain of said reference antibodies:
1. MGU10variant1 (MGU10v1), which differs from MGU10 in the framework regions (FR) of the heavy chain variable region (VH; SEQ ID NO: 11);
2. MGU10variant2 (MGU10v2), which differs from MGU10 in the heavy chain CDR3 (CDRH3: SEQ ID NO: 12; VH: SEQ ID NO: 13);
3. MGU10variant3 (MGU10v3), which differs from MGU10 in the light chain CDR3 (CDRL3: SEQ ID NO: 14; light chain variable region (VL): SEQ ID NO: 15);
4. MGU10variant4 (MGU10v4), which represents a combination of MGU10v1 and MGU10v2 and, therefore, differs from MGU10 in the heavy chain CDR3 (CDRH3; SEQ ID NO: 12) and in the heavy chain FR (VH; SEQ ID NO: 16);
5. MGU10variant5 (MGU10v5), which represents a combination of MGU10v1 and MGU10v3 (including the VH of MGU10v1 and the VL of MGU10v3) and, therefore, differs from MGU10 in the heavy chain FR (VH; SEQ ID NO: 11) and in the CDRL3 (CDRL3: SEQ ID NO: 14; VL: SEQ ID NO: 15);
6. MGU10variant6 (MGU10v6), which represents a combination of MGU10v2 and MGU10v3 (including the VH of MGU10v2 and the VL of MGU10v3) and, therefore, differs from MGU10 in the CDRH3 (CDRH3: SEQ ID NO: 12; VH: SEQ ID NO: 13) and in the CDRL3 (CDRL3: SEQ ID NO: 14; VL: SEQ ID NO: 15);
7. MGU10variant7 (MGU10v7), which represents a combination of MGU10v1, MGU10v2 and MGU10v3 (including the VH of MGU10v4 and the VL of MGU10v3) and, therefore, differs from MGU10 in the CDRH3 (SEQ ID NO: 12) and heavy chain FR (VH; SEQ ID NO: 16) and in the CDRL3 (CDRL3: SEQ ID NO: 14; VL: SEQ ID NO: 15); and
8. MGH2variant1 (MGH2v1), which differs from MGH2 in the light chain CDR3 (CDRL3: SEQ ID NO: 28; VL: SEQ ID NO: 29).

An overview over the SEQ ID NOs of CDR and VH/VL sequences of the variants in comparison with MGU10 and MGH2, respectively, is provide in Table 2:

TABLE 2

| Antibody | Heavy chain | | | | Light chain | | | |
|---|---|---|---|---|---|---|---|---|
| name | CDR1 | CDR2 | CDR3 | VH | CDR1 | CDR2 | CDR3 | VL |
| MGU10 | 1 | 2 | 3 | 7 | 4 | 5 | 6 | 8 |
| MGU10v1 | 1 | 2 | 3 | 11 | 4 | 5 | 6 | 8 |
| MGU10v2 | 1 | 2 | 12 | 13 | 4 | 5 | 6 | 8 |
| MGU10v3 | 1 | 2 | 3 | 7 | 4 | 5 | 14 | 15 |
| MGU10v4 | 1 | 2 | 12 | 16 | 4 | 5 | 6 | 8 |
| MGU10v5 | 1 | 2 | 3 | 11 | 4 | 5 | 14 | 15 |
| MGU10v6 | 1 | 2 | 12 | 13 | 4 | 5 | 14 | 15 |
| MGU10v7 | 1 | 2 | 12 | 16 | 4 | 5 | 14 | 15 |
| MGH2 | 17 | 18 | 19 | 24 | 20 | 21/22 | 23 | 25 |
| MGH2v1 | 17 | 18 | 19 | 24 | 20 | 21/22 | 28 | 29 |

### Example 2: Antibodies of the Invention Show Dual Specificity

Tan et al., 2018 showed that the most potent antibodies of their study—including MGU10 and MGH2— simultaneously target epitopes in (i) the NANP-repeat region of CSP and (ii) an N-terminal region of CSP covering the junction between the N-terminal domain and the NANP-repeats (Tan), Sack B K, Oyen D, et al. A public antibody lineage that potently inhibits malaria infection through dual binding to the circumsporozoite protein. Nat Med. 2018; 24(4):401-407. doi:10.1038/nm.4513). Moreover, this study showed that the extreme potency of those antibodies was due to their dual specificity, while antibodies targeting only one of the CSP epitopes were typically less potent.

Accordingly, antibodies of the present invention were produced and tested for their capacity to bind to both of the epitopes described by Tan et al., 2018: (i) the NANP-repeat region of CSP and (ii) the N-terminal region of CSP covering the junction between the N-terminal domain and the NANP-repeats.

To this end, antibodies MGU10v1, MGU10v2, MGU10v3 and MGH2v1 were produced. Namely, the antibodies were synthetized by Genscript and subcloned in vectors for the expression of IgG1 and kappa or lambda chains. The purified plasmids of heavy and light chains were combined and used to transfect Expi293F cells (ThermoFisher Scientific) using polyethylenimine (micro-scale transfection (600 μl) in a 96-well plate). The transfected cells were harvested on day 6 and supernatants were collected by centrifugation and filtration.

Total IgGs present in the supernatants were quantified using 96-well MaxiSorp plates (Nunc) coated with 10 μg/ml goat anti-human IgG (SouthernBiotech). Plates were then blocked with PBS with 1% BSA and incubated with titrated monoclonal antibodies, using Certified Reference Material 470 (ERMs-DA470, Sigma-Aldrich) as a standard. Plates were then washed and incubated with 1/500 alkaline phosphatase (AP)-conjugated goat anti-human IgG (Southern Biotech). Substrate (para-nitrophenyl phosphate (p-NPP), Sigma) was added and plates were read at wavelength of 405 nm to determine optical density (OD) values.

To test specific antibody binding to (i) the NANP-repeat region of CSP and (ii) the N-terminal region of CSP covering the junction between the N-terminal domain and the NANP-repeats, ELISA plates were coated with 10 μg/ml of avidin (Sigma). Plates were blocked with PBS with 1% BSA and incubated with 1 μg/ml biotinylated NANP-peptide (SEQ ID NO: 34) or with 1 μg/ml biotinylated NPDP-peptide (SEQ ID NO: 35). The plates were washed and incubated with titrated monoclonal antibodies, followed by 1/500 AP-conjugated goat anti-human IgG (Southern Biotech) and pNPP substrate. EC50 (ng/ml) values were calculated for every sample tested by nonlinear regression analysis using the Graph Pad Prism 7 software.

The results are shown in Table 3 for MGU10 and its variants MGU10v1, MGU10v2 and MGU10v3 and in Table 4 for MGH2 and its variant MGH2v1:

TABLE 3

| Antibody | NANP EC₅₀ (ng/ml) | NPDP EC₅₀ (ng/ml) |
|---|---|---|
| MGU10 | 34.3 | 26.1 |
| MGU10v1 | 28.51 | 22.54 |
| MGU10v2 | 26.79 | 20.45 |
| MGU10v3 | 26.28 | 25.08 |

TABLE 4

| Antibody | NANP EC₅₀ (ng/ml) | NPDP EC₅₀ (ng/ml) |
|---|---|---|
| MGH2 | 27.88 | 48.8 |
| MGH2v1 | 22.32 | 65.82 |

Surprisingly, all variants tested for MGU10 show an increased binding affinity for both CSP epitopes, while MGH2v1 only shows an increased binding affinity for the NANP-repeat region in comparison to MGH2.

### Example 3: Sporozoite Invasion/Maturation Assay

Variant antibody MGU10v2 was selected for further characterization in functional assays. The sporozoite invasion/maturation assay is a functional assay for testing the antibodies' effects on the infectivity of sporozoites. Sporozoite invasion of hepatocytes and subsequent maturation into exoerythrocytic forms is an essential step in the establishment of malaria infection.

Cryopreserved human primary hepatocytes were seeded in a microtiter plate and incubated for 2 days. Salivary gland Plasmodium falciparum NF54 sporozoites were isolated from An. stephensi mosquitoes infected with P. falciparum. For each well, sporozoites were pre-incubated with serially diluted antibody sample for 30 minutes and thereafter transferred onto the hepatocytes. After 3 hours, non-invaded sporozoites were washed off and the cells were incubated for 4 days. Cells were fixed and stained with anti-HSP70 and DAPI. The number of hepatocyte nuclei and HSP70 positive forms were quantified by automated high content imaging.

In this assay, variant antibody MGU10v2 was compared to the parental antibody MGU10. In addition, Fc variant “MGU10v2_LS” of MGU10v2 was tested, which differs from variant antibody MGU10v2 only in that it comprises the mutations M428L and N434S (EU numbering) in the heavy chain constant region (amino acid sequence of MGU10v2_LS heavy chain: SEQ ID NO: 100). Accordingly, the variable regions of MGU10v2_LS are identical to those of MGU10v2. An irrelevant antibody was used as control. Five dilutions were tested per antibody sample with two replicates per dilution. 3SP2/Atovaquone treated sporozoites were used as MIN control and vehicle treated sporozoites as MAX control.

Data are expressed as total number of hepatocyte nuclei, total number of HSP70 positive forms, % infected hepatocytes. IC50 values were estimated using a four parameter non-linear regression model using least squares to find the best fit. The resulting values (expressed in μg/ml) are presented in Table 5 below:

TABLE 5

| Antibody | IC50 (μg/ml) |
|---|---|
| MGU10 | 0.418 |
| MGU10v2 | 0.302 |
| MGU10v2_LS | 0.179 |
| control | >10 |

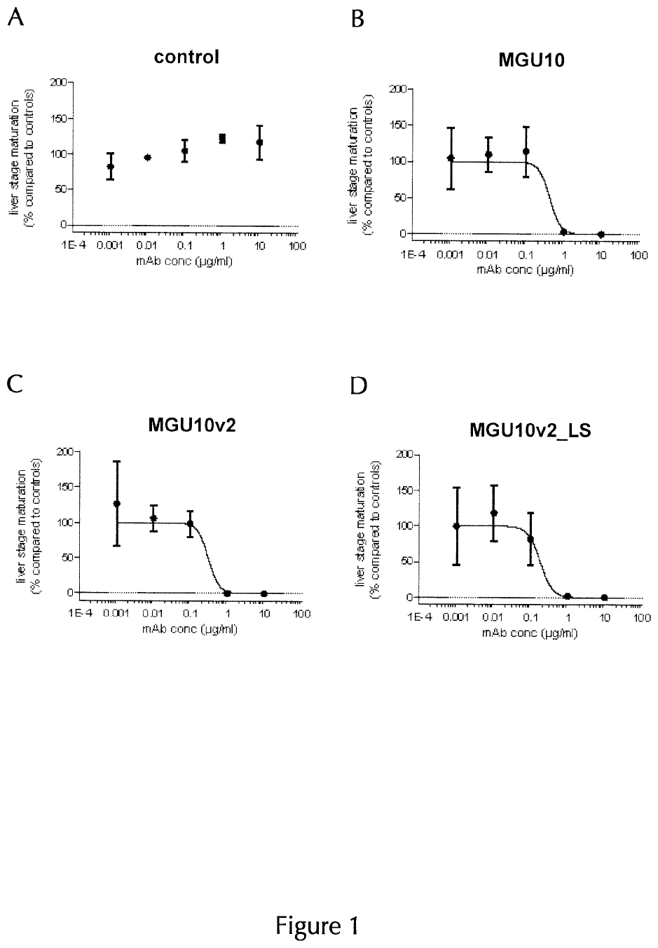

Graphical representations of the results are shown in FIG. 1. All antibodies, with the exception of the control antibody, functionally blocked the development of liver stage schizonts in human primary hepatocytes. Interestingly, variant antibody MGU10v2 was more effective than parental antibody MGU10 and the additional Fc variant MGU10v2_LS was most efficient.

### Example 4: Sporozoite Gliding Assay

The sporozoite gliding assay is a functional assay, wherein the effects of compounds on the sporozoites' gliding motility can be assessed. Plasmodium sporozoites are transmitted into the skin of their vertebrate host through the bite of an infectious mosquito. Sporozoite motility is a key prerequisite for parasite transmission and successful infection of the vertebrate host. Motility constitutes the first parasite mechanism that can be inhibited and is, therefore, of interest for intervention strategies.

Plates were coated with anti-CSP mAb 3SP2 to capture shed CSP (circumsporozoite protein). Fresh salivary gland sporozoites were isolated from An. stephensi mosquitoes infected with P. falciparum and pre-incubated with serially diluted sample (5 dilutions/sample) for 30 min and then transferred into the 3SP2 coated wells. After 90 minutes sporozoites were washed off and gliding trails were fixed and stained with biotinylated-anti-CSP-antibody followed by streptavidin-AF555. Gliding trails were captured by automated high content imaging and total gliding trail length was analyzed using machine learning algorithms.

In this assay, variant antibody MGU10v2_LS was compared to the parental antibody MGU10. An irrelevant antibody was used as control. Five dilutions were tested per antibody sample with two replicates per dilution. 3SP2/Gramicidin treated sporozoites were used as MIN control and vehicle treated sporozoites as MAX control.

Total gliding trails were quantified by image analysis and reported as relative fluorescence counts. IC50 values were estimated using a four parameter non-linear regression model using least squares to find the best fit. The resulting values (expressed in μg/ml) are presented in Table 6 below:

TABLE 6

| Antibody | IC50 (μg/ml) |
|---|---|
| MGU10 | 0.975 |
| MGU10v2_LS | 0.748 |
| control | >10 |

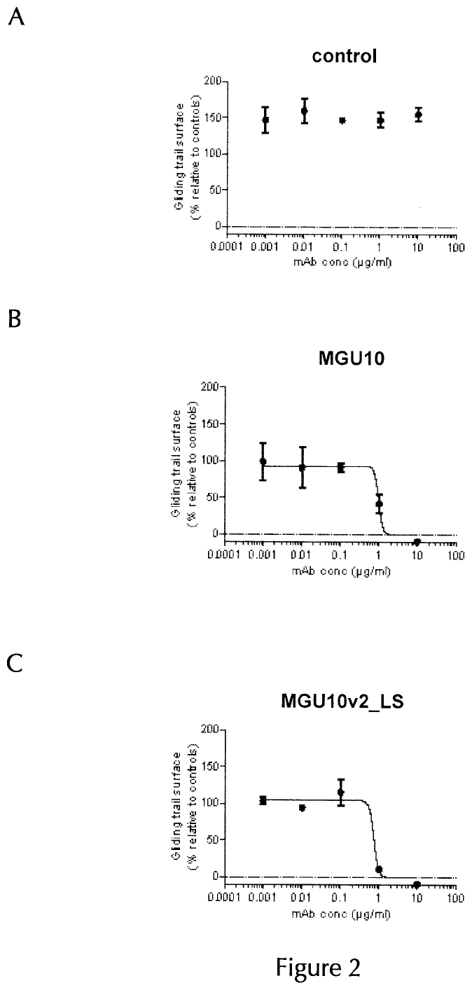

Graphical representations of the results are shown in FIG. 2. Antibodies MGU10 and MGU10v2_LS, but not the control antibody, functionally blocked gliding motility of P. falciparum sporozoites. Variant antibody MGU10v2_LS was more effective than parental antibody MGU10.

### Example 5: Sporozoite Traversal Assay

Plasmodium sporozoites are deposited in the skin of the vertebrate host. As sporozoites move towards the liver, they can enter and exit host cells within transient vacuoles, a process known as cell traversal. Traversal allows the sporozoites to cross cellular barriers and evade the host immune response, thereby representing a key prerequisite for successful infection of the vertebrate host. The sporozoite traversal assay is a functional assay, wherein the effects of compounds on the sporozoites' cell traversal can be assessed.

Human hepatoma (HC-04) cells were seeded in microtiter plates and grown to near confluence. Fresh P. falciparum salivary gland sporozoites were isolated from An. stephensi mosquitoes and pre-incubated with diluted IgG for 30 minutes before adding rhodamin-dextran. Following incubation for 1 hour at 37° C., cell nuclei were stained with DAPI. Fluorescence levels of traversed cells were quantified using a high content automated imager.

In this assay, variant antibody MGU10v2_LS was compared to the parental antibody MGU10. An irrelevant antibody was used as control. Five dilutions were tested per antibody sample with two replicates per dilution. 3SP2/Cytochalasin D treated sporozoites were used as MIN control and vehicle treated sporozoites as MAX control.

Data were expressed as % traversed cells relative to the MIN and MAX controls of the assay plate. IC50 values were estimated using a four parameter non-linear regression model using least squares to find the best fit. The resulting values (expressed in μg/ml) are presented in Table 7 below:

TABLE 7

| Antibody | IC50 (μg/ml) |
|---|---|
| MGU10 | 1.928 |
| MGU10v2_LS | 1.356 |
| control | >10 |

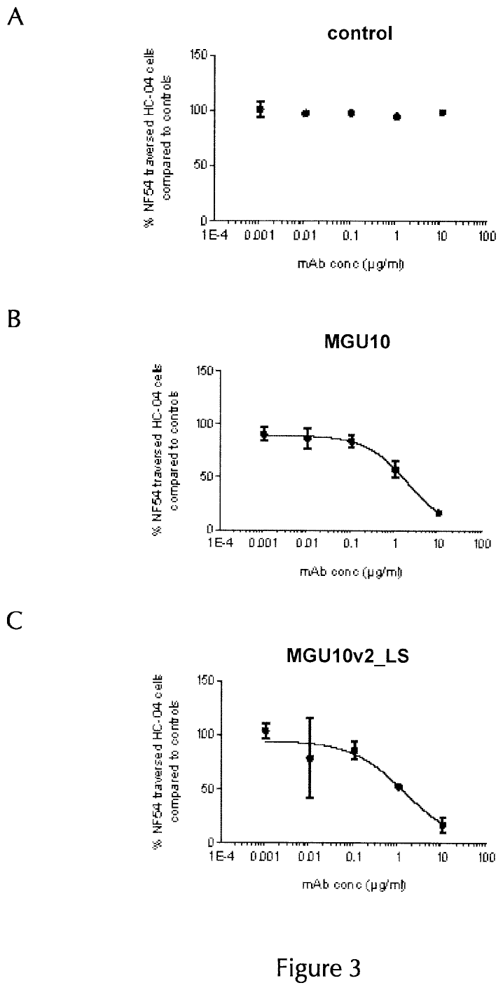

Graphical representations of the results are shown in FIG. 3. Antibodies MGU10 and MGU10v2_LS, but not the control antibody, functionally blocked traversal of P. falciparum sporozoites. Variant antibody MGU10v2_LS was more effective than parental antibody MGU10.

### Example 6: Stability of Antibodies

To test their stability, variant antibody MGU10v2_LS was compared to its parental version MGU10_LS. MGU10_LS differs from parental antibody MGU10 only in that it comprises the mutations M428L and N434S (EU numbering) in the heavy chain constant region (amino acid sequence of MGU10_LS heavy chain: SEQ ID NO: 101) Accordingly, the variable regions of MGU10_LS are identical to those of MGU10. Variant antibody MGU10v2_LS and its parental version MGU10_LS were exposed to heat stress under different conditions.

To this end, variant antibody MGU10v2_LS and its parental version MGU10_LS were incubated at 40° C. in sodium acetate buffer at pH 5.6 for two weeks. The formation of aggregates and dimers (high and low molecular weight species) was assessed by size exclusion chromatography. Results are shown in Table 8 below:

TABLE 8

| | MGU10_LS | | | MGU10v2_LS | | |
|---|---|---|---|---|---|---|
| | % HMWS | % monomer | % LMWS | % HMWS | % monomer | % LMWS |
| unstressed | 0.63 | 99 | 0.37 | 0.22 | 99.38 | 0.4 |
| 40° C. | 4.65 | 92.83 | 2.52 | 4.29 | 93.09 | 2.62 |

HMWS: high molecular weight species, indicating aggregation; LMWS: low molecular weight species, representing dimer formation; % monomer indicates antibodies which do not dimerize or aggregate.

To further assess stability and aggregation of mAb MGU10, parental and developed mAbs were tested in two different buffers with different pH. To compare mAb dimerization and aggregation in different buffers, mAbs batches were buffer-exchanged to 50 mM Na-Acetate, 50 mM NaCl, pH 5.5 or to 20 mM Na-Citrate, 50 mM NaCl, pH 6.0 upon purification. Size exclusion chromatography was used to assess the molecular weight of the mAb species after 4 and 15 days at 40° C., using a BEH450 SEC Protein Standard Mix 5 component protein mixture (Thyroglobulin, IgG, BSA, Myoglobin, Uracil) for size calculation.

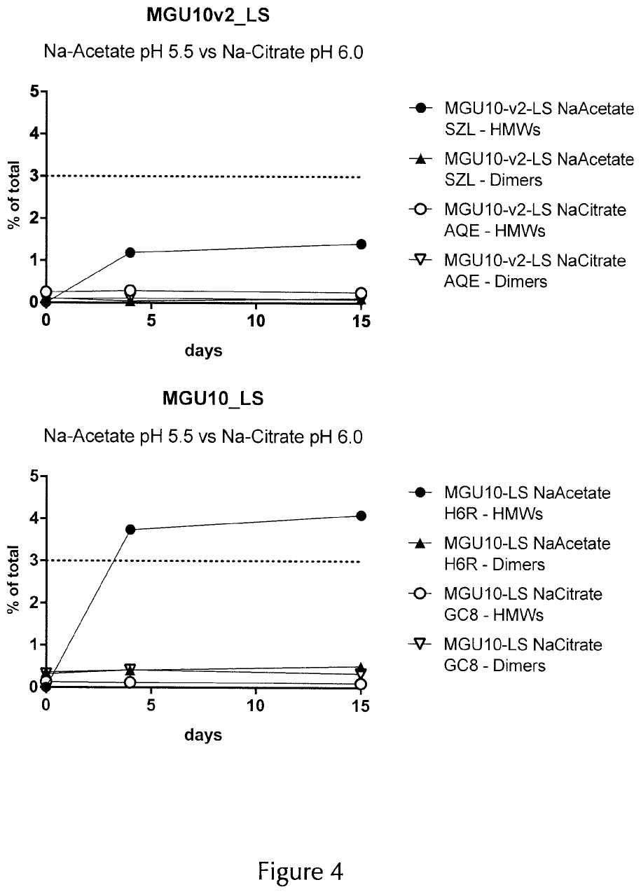

Results are shown in FIG. 4. Variant antibody MGU10v2_LS formed less high molecular weight species (less aggregates) when kept at 40° C. in 50 mM Na-Acetate/50 mM NaCl buffer, pH 5.5, for two weeks as compared to its parental version MGU10_LS. Accordingly, variant antibody MGU10v2_LS shows increased stability compared to the parental antibody.

### Example 7: In Vivo Protection Conferred by Variant Antibody MGU10v2— Challenge Study

To assess in vivo protection against P. berghei chimeric parasite expressing full-length P. falciparum CSP, C57BL/6 mice (n=5 per group) were injected i.v. with 54.5 μg/mouse of variant antibody MGU10v2_LS or with 100 μg/mouse of the respective parental antibody MGU10_LS. As negative control, unrelated antibody AB-1245 (100 μg/mouse) was used. Forty-eight hours after antibody injection, antibody-treated mice as well as an additional group of naive mice (n=5) were challenged with 2×10³ chimeric P. berghei sporozoites expressing full-length P. falciparum CSP injected i.v. Forty-two hours after challenge, mice were injected with 100 μl of D-Luciferin (30 mg/mL), anesthetized with isoflurane and imaged with the IVIS spectrum to measure the bioluminescence expressed by the chimeric parasites. % inhibition was calculated in comparison to the naive group (representing 100% infection).

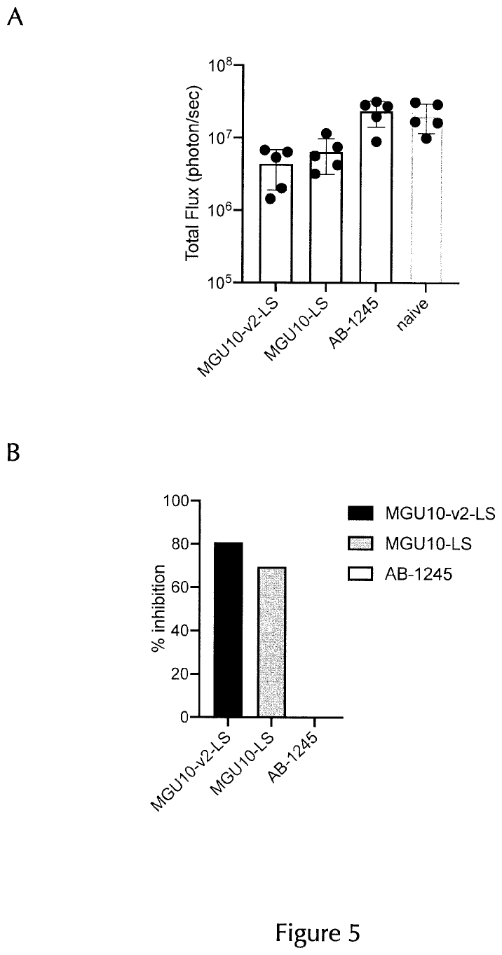

Results are shown in FIG. 5. In contrast to unrelated control antibody AB-1245, both variant antibody MGU10v2_LS and the respective parental antibody MGU10_LS significantly inhibited infection with the parasite in vivo with 80.76 (MGU10v2_LS) and 69.60% inhibition (MGU10_LS), respectively. Accordingly, a stronger inhibition of infection was achieved with variant antibody MGU10v2_LS as compared to its parental antibody MGU10_LS.

### Example 8: In Vivo Protection Conferred by Variant Antibody MGH2v1— Challenge Study

To assess in vivo protection against P. berghei chimeric parasite expressing full-length P. falciparum CSP, C57BL/6 mice (n=5 per group) were injected i.v. with 100 μg/mouse of variant antibody MGH2v1_LS or with the respective parental antibody MGH2_LS. Fc variants MGH2v1_LS and MGH2_LS differ from MGH2v1 and MGH2, respectively, only in two mutations in the Fc region, i.e. the respective variable regions are maintained. Namely, variant antibody MGH2v1_LS differs from variant antibody MGH2v1 only in that it comprises the mutations M428L and N434S (EU numbering) in the heavy chain constant region (amino acid sequence of MGH2v1_LS heavy chain: SEQ ID NO: 102). Accordingly, antibody MGH2_LS differs from antibody MGH2 only in that it comprises the mutations M428L and N434S (EU numbering) in the heavy chain constant region (amino acid sequence of MGH2_LS heavy chain: SEQ ID NO: 102). As variant antibody MGH2v1 differs from parental antibody MGH2 only in the light chain CDR3 (VL), the heavy chain amino acid sequences of MGH2LS and MGH2v1_LS are identical.

As negative control, unrelated antibody AB-1245 (100 μg/mouse) was used. Forty-eight hours after antibody injection, antibody-treated mice as well as an additional group of naive mice (n=5) were challenged with 2×10³ chimeric P. berghei sporozoites expressing full-length P. falciparum CSP injected i.v. as described in Example 7. Forty-two hours after challenge, mice were injected with 100 μl of D-Luciferin (30 mg/mL), anesthetized with isoflurane and imaged with the IVIS spectrum to measure the bioluminescence expressed by the chimeric parasites. % inhibition was calculated in comparison to the naive group (representing 100% infection).

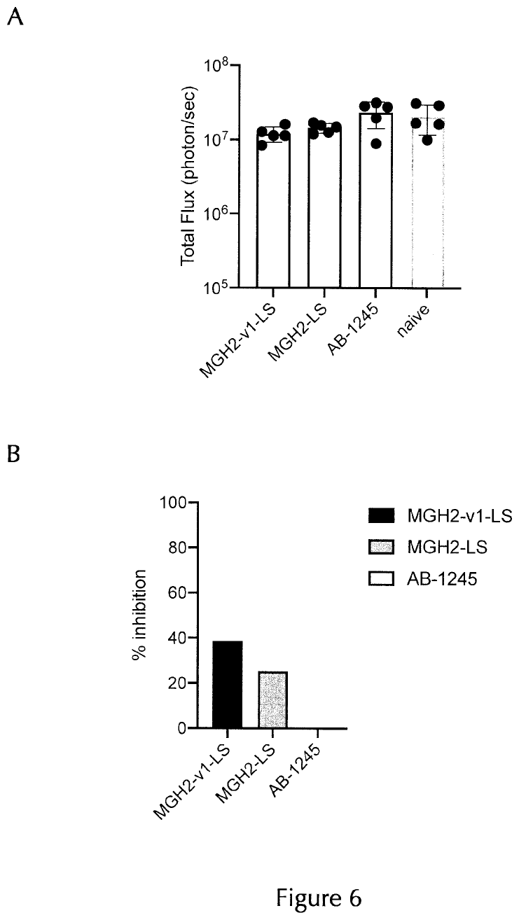

Results are shown in FIG. 6. In contrast to unrelated control antibody AB-1245, both variant antibody MGH2v1_LS and the respective parental antibody MGH2_LS inhibited infection with the parasite in vivo with 38.62% (MGH2v1_LS) and 25.23% inhibition (MGH2_LS), respectively. Accordingly, a stronger inhibition of infection was achieved with variant antibody MGH2v1_LS as compared to its parental antibody MGH2v1_LS.

### Example 9: Design and Testing of a Further Variant of MGU10/MGU10v2

As shown above in Example 6, MGU10v2, which differs from MGU10 in mutation D106E, shows increased stability. Without being bound to any theory, the present inventors believe that mutation D106E removes an isomerization motif and thereby increases the stability of antibody MGU10v2 as shown in Example 6.

In view thereof, a further variant of MGU10 was designed, wherein—in comparison to MGU10v2— the additional mutation D97E was introduced in the framework region of the heavy chain (VH) of MGU10v2 in order to remove a further isomerization motif (to further increase the antibody's stability and manufacturability). Accordingly, MGU10v8 comprises the same CDR sequences as MGU10v2; and the same VL sequence as MGU10v2 and its parental antibody MGU10. The VH of MGU10v8 comprises an amino acid sequence as set forth in SEQ ID NO: 104. SEQ ID NO: 106 provides an exemplary nucleotide sequence encoding the MGHv8 VH.

New variant antibody MGU10v8 was expressed essentially as described in Example 2 using ExpiCHO cells (in higher volume, e.g. 25 ml or 100 ml or more), and binding to (i) the NANP-repeat region of CSP (“NANP”-peptide) and (ii) the N-terminal region of CSP covering the junction between the N-terminal domain and the NANP-repeats (“NPDP”-peptide) were tested in ELISA. To this end, Pierce™ Streptavidin coated plates (Life Technologies) were used to coat either biotinylated NANP-peptide (N-term biotinylation; SEQ ID NO: 34) or biotinylated NPDP19-peptide (N-term biotinylation; SEQ ID NO: 105), each at 5 μg/ml in blocking buffer (PBS, 1% BSA). Plates were washed (PBS, 0.05% Tween 20) before addition of titrated antibodies MGU10v2 or MGU10v8 for 90 min at RT. After another wash step, secondary antibody goat anti-human IgG HRP F(ab′)2 fragment Fcg specific (Jackson ImmunoResearch) was added at 0.8 μg/ml. Sure Blue TMB (Bioconcept) was used for color development that was stopped with 1% HCl in water. Optical density at 450 nm was detected using an ELISA Reader ELx808IU (Biotek).

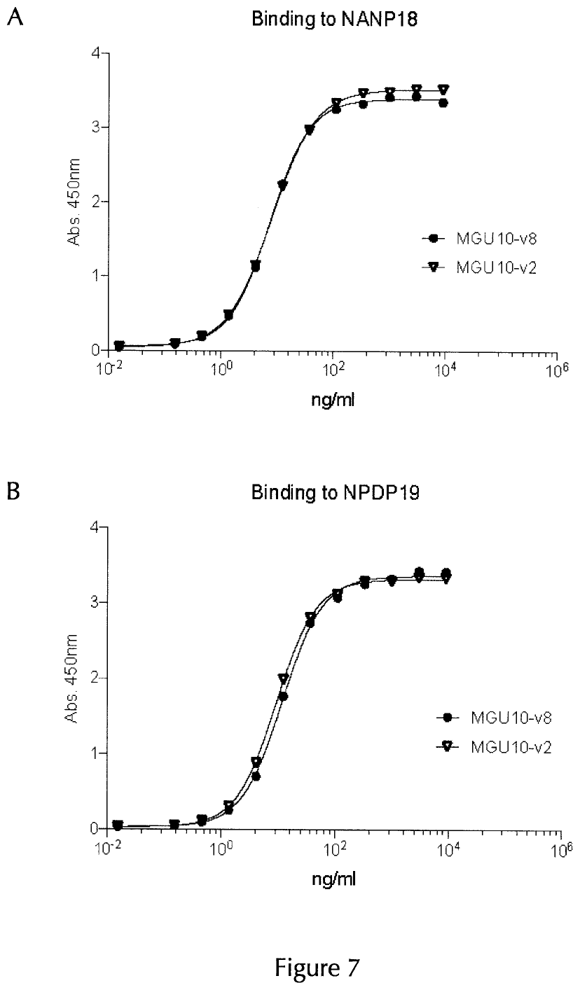

Results are shown in FIG. 7. As shown in FIG. 7, binding to both peptides, NANP and NPDP19, is very similar in variant antibodies MGU10v2 and MGU10v8.

TABLE OF SEQUENCES AND SEQ ID NUMBERS (SEQUENCE LISTING):

| | | |
|---|---|---|
| SEQ ID NO | Sequence | Remarks |

Amino acid sequences
MGU10

| | | |
|---|---|---|
| SEQ ID NO: 1 | GFAFSNYG | CDRH1 |
| SEQ ID NO: 2 | IWHDGSLK | CDRH2 |
| SEQ ID NO: 3 | TVWYLETPDDGFDI | CDRH3 |
| SEQ ID NO: 4 | HGHTSKA | CDRL1 |
| SEQ ID NO: 5 | VNSDGSH | CDRL2 |
| SEQ ID NO: 6 | QAWDSGIWV | CDRL3 |
| SEQ ID NO: 7 | QVQLVESGGGVVQPGRSLRLSCAASGFAFSNY | VH |
| | GMNWVRQAPGKGLEWVAVIWHDGSLKYYTQ | |
| | SVKGRFTISRDNAKNTLFLQMDSLSADDTAMYY | |
| | CTVWYLETPDDGFDIWGRGTMVTVSS | |
| SEQ ID NO: 8 | QLVLTQPPSASASLGVSVTLTCTLSHGHTSKAIA | VL |
| | WHQQQPGKGPRYLMKVNSDGSHTKGAAVPD | |
| | RFSGSTSGAERHFTISNLQSDDEADYYCQAWDS | |
| | GIWVFGGGTKLTVL | |
| SEQ ID NO: 9 | QVQLVESGGGVVQPGRSLRLSCAASGFAFSNY | Heavy chain |
| | GMNWVRQAPGKGLEWVAVIWHDGSLKYYTQ | |
| | SVKGRFTISRDNAKNTLFLQMDSLSADDTAMYY | |
| | CTVWYLETPDDGFDIWGRGTMVTVSSASTKGP | |
| | SVFPLAPSSKSTSGGTAALGCLVKDYFPEPVTVS | |
| | WNSGALTSGVHTFPAVLQSSGLYSLSSVVTVPSS | |
| | SLGTQTYICNVNHKPSNTKVDKKVEPKSCDKTH | |
| | TCPPCPAPELLGGPSVFLFPPKPKDTLMISRTPEVT | |
| | CVVVDVSHEDPEVKFNWYVDGVEVHNAKTKPR | |
| | EEQYNSTYRVVSVLTVLHQDWLNGKEYKCKVS | |
| | NKALPAPIEKTISKAKGQPREPQVYTLPPSRDELTK | |
| | NQVSLTCLVKGFYPSDIAVEWESNGQPENNYKT | |
| | TPPVLDSDGSFFLYSKLTVDKSRWQQGNVFSCS | |
| | VLHEALHSHYTQKSLSLSPGK | |
| SEQ ID NO: 10 | QLVLTQPPSASASLGVSVTLTCTLSHGHTSKAIA | Light chain |
| | WHQQQPGKGPRYLMKVNSDGSHTKGAAVPD | |
| | RFSGSTSGAERHFTISNLQSDDEADYYCQAWDS | |
| | GIWVFGGGTKLTVLGQPKAAPSVTLFPPSSEELQ | |
| | ANKATLVCLISDFYPGAVTVAWKADSSPVKAGV | |
| | ETTTPSKQSNNKYAASSYLSLTPEQWKSHRSYSC | |
| | QVTHEGSTVEKTVAPTECS | |

MGU10variant1 (MGU10v1)

| | | |
|---|---|---|
| SEQ ID NO: 11 | QVQLVESGGGVVQPGRSLRLSCAASGFAFSNY | VH |
| | GMHWVRQAPGKGLEWVAVIWHDGSLKYYTQ | |
| | SVKGRFTISRDNAKNTLFLQMDSLSADDTAMYY | |
| | CTVWYLETPDDGFDIWGQGTMVTVSS | |

MGU10variant2 (MGU10v2)

| | | |
|---|---|---|
| SEQ ID NO: 12 | TVWYLETPDEGFDI | CDRH3 |
| SEQ ID NO: 13 | QVQLVESGGGVVQPGRSLRLSCAASGFAFSNY | VH |
| | GMNWVRQAPGKGLEWVAVIWHDGSLKYYTQ | |
| | SVKGRFTISRDNAKNTLFLQMDSLSADDTAMYY | |
| | CTVWYLETPDEGFDIWGRGTMVTVSS | |

MGU10variant3 (MGU10v3)

| | | |
|---|---|---|
| SEQ ID NO: 14 | QAWESGIWV | CDRL3 |
| SEQ ID NO: 15 | QLVLTQPPSASASLGVSVTLTCTLSHGHTSKAIA | VL |
| | WHQQQPGKGPRYLMKVNSDGSHTKGAAVPD | |
| | RFSGSTSGAERHFTISNLQSDDEADYYCQAWES | |
| | GIWVFGGGTKLTVL | |

MGU10variant4 (MGU10v4)

| | | |
|---|---|---|
| SEQ ID NO: 16 | QVQLVESGGGVVQPGRSLRLSCAASGFAFSNY | VH |
| | GMHWVRQAPGKGLEWVAVIWHDGSLKYYTQ | |
| | SVKGRFTISRDNAKNTLFLQMDSLSADDTAMYY | |
| | CTVWYLETPDEGFDIWGQGTMVTVSS | |

MGH2

| | | |
|---|---|---|
| SEQ ID NO: 17 | GFSFSSYA | CDRH1 |
| SEQ ID NO: 18 | TRYDGSNK | CDRH2 |
| SEQ ID NO: 19 | AKVGDGTVAGTIDY | CDRH3 |
| SEQ ID NO: 20 | QSLVYSDGNTY | CDRL1 |
| SEQ ID NO: 21 | KVS | CDRL2 |
| SEQ ID NO: 22 | LIYKVSNRD | CDRL2 long |
| SEQ ID NO: 23 | MQGTHWWT | CDRL3 |
| SEQ ID NO: 24 | QVQLVESGGGVVQPGGSLRLSCTASGFSFSSYA | VH |
| | MHWVRQAPGKGLEWVAYTRYDGSNKFYLDSV | |
| | QGRFTISRDNSKNTLYLEMDSLRLEDTAVYFCAK | |
| | VGDGTVAGTIDYWGQGTLVTVSS | |
| SEQ ID NO: 25 | YIVMTQSPLSLPVTLGQPASISCRSSQSLVYSDGN | VL |
| | TYLNWYQQRPGQSPRRLIYKVSNRDSGVPDRFS | |
| | GSGSGTDFTLKISRVEAEDVGVYYCMQGTHW | |
| | WTFGQGTKVEIK | |
| SEQ ID NO: 26 | QVQLVESGGGVVQPGGSLRLSCTASGFSFSSYA | Heavy chain |
| | MHWVRQAPGKGLEWVAYTRYDGSNKFYLDSV | |
| | QGRFTISRDNSKNTLYLEMDSLRLEDTAVYFCAK | |
| | VGDGTVAGTIDYWGQGTLVTVSSASTKGPSVF | |
| | PLAPSSKSTSGGTAALGCLVKDYFPEPVTVSWNS | |
| | GALTSGVHTFPAVLQSSGLYSLSSVVTVPSSSLGT | |
| | QTYICNVNHKPSNTKVDKKVEPKSCDKTHTCPP | |
| | CPAPELLGGPSVFLFPPKPKDTLMISRTPEVTCVV | |
| | VDVSHEDPEVKFNWYVDGVEVHNAKTKPREEQ | |
| | YNSTYRVVSVLTVLHQDWLNGKEYKCKVSNKA | |
| | LPAPIEKTISKAKGQPREPQVYTLPPSRDELTKNQ | |
| | VSLTCLVKGFYPSDIAVEWESNGQPENNYKTTPP | |
| | VLDSDGSFFLYSKLTVDKSRWQQGNVFSCSVLH | |
| | EALHSHYTQKSLSLSPGK | |
| SEQ ID NO: 27 | YIVMTQSPLSLPVTLGQPASISCRSSQSLVYSDGN | Light chain |
| | TYLNWYQQRPGQSPRRLIYKVSNRDSGVPDRFS | |
| | GSGSGTDFTLK1SRVEAEDVGVYYCMQGTHW | |
| | WTFGQGTKVEIKRTVAAPSVFIFPPSDEQLKSGT | |
| | ASVVCLLNNFYPREAKVQWKVDNALQSGNSQE | |
| | SVTEQDSKDSTYSLSSTLTLSKADYEKHKVYACEV | |
| | THQGLSSPVTKSFNRGEC | |

MGH2variant1 (MGH2v1)

| | | |
|---|---|---|
| SEQ ID NO: 28 | MQGTHFWT | CDRL3 |
| SEQ ID NO: 29 | YIVMTQSPLSLPVTLGQPASISCRSSQSLVYSDGN | VL |
| | TYLNWYQQRPGQSPRRLIYKVSNRDSGVPDRFS | |
| | GSGSGTDFTLKISRVEAEDVGVYYCMQGTHFW | |
| | TFGQGTKVEIK | |

Constant regions

| | | |
|---|---|---|
| SEQ ID NO: 30 | ASTKGPSVFPLAPSSKSTSGGTAALGCLVKDYFPE | heavy chain constant |
| | PVTVSWNSGALTSGVHTFPAVLQSSGLYSLSSVV | region |
| | TVPSSSLGTQTYICNVNHKPSNTKVDKKVEPKSC | |
| | DKTHTCPPCPAPELLGGPSVFLFPPKPKDTLMISR | |
| | TPEVTCVVVDVSHEDPEVKFNWYVDGVEVHNA | |
| | KTKPREEQYNSTYRVVSVLTVLHQDWLNGKEYK | |
| | CKVSNKALPAPIEKTISKAKGQPREPQVYTLPPSR | |
| | DELTKNQVSLTCLVKGFYPSDIAVEWESNGQPE | |
| | NNYKTTPPVLDSDGSFFLYSKLTVDKSRWQQG | |
| | NVFSCSVLHEALHSHYTQKSLSLSPGK | |
| SEQ ID NO: 31 | GQPKAAPSVTLFPPSSEELQANKATLVCLISDFYP | lambda light chain |
| | GAVTVAWKADSSPVKAGVETTTPSKQSNNKYA | |
| | ASSYLSLTPEQWKSHRSYSCQVTHEGSTVEKTVA | |
| | PTECS | |
| SEQ ID NO: 32 | RTVAAPSVFIFPPSDEQLKSGTASVVCLLNNFYPR | kappa light chain |
| | EAKVQWKVDNALQSGNSQESVTEQDSKDSTYS | |
| | LSSTLTLSKADYEKHKVYACEVTHQGLSSPVTKSF | |
| | NRGEC | |
| SEQ ID NO: 33 | MMRKLAILSVSSFLFVEALFQEYQCYGSSSNTRVL | PfCSP |
| | NELNYDNAGTNLYNELEMNYYGKQENWYSLK | |
| | KNSRSLGENDDGNNEDNEKLRKPKHKKLKQPA | |
| | DGNPDPNANPNVDPNANPNVDPNANPNVDP | |
| | NANPNANPNANPNANPNANPNANPNANPNA | |
| | NPNANPNANPNANPNANPNANPNANPNANP | |
| | NANPNANPNVDPNANPNANPNANPNANPNA | |
| | NPNANPNANPNANPNANPNANPNANPNANP | |
| | NANPNANPNANPNANPNANPNANPNKNNQ | |
| | GNGQGHNMPNDPNRNVDENANANSAVKNN | |
| | NNEEPSDKHIKEYLNKIQNSLSTEWSPCSVTCGN | |
| | GIQVRIKPGSANKPKDELDYANDIEKKICKMEKC | |
| | SSVFNVVNSSIGLIMVLSFLFLN | |
| SEQ ID NO: 34 | NANPNANPNANPNANPNA | NANP-peptide |
| SEQ ID NO: 35 | KQPADGNPDPNANPN | NPDP-peptide |

Nucleic acid sequences

| | | |
|---|---|---|
| SEQ ID NO: 36 | CAGGTGCAGCTGGTGGAGAGCGGAGGAGG | MGU10v1 VH |
| | AGTGGTGCAGCCAGGCCGGTCCCTGAGACT | |
| | GTCTTGCGCCGCCAGCGGCTTCGCCTTTTCC | |
| | AACTACGGAATGCACTGGGTGCGGCAGGCA | |
| | CCTGGCAAGGGACTGGAGTGGGTGGCCGTG | |
| | ATCTGGCACGACGGATCCCTGAAGTACTATA | |
| | CACAGTCTGTGAAGGGCAGGTTCACCATCTC | |
| | TCGCGACAACGCCAAGAATACACTGTTTCTG | |
| | CAGATGGATTCTCTGAGCGCCGACGATACCG | |
| | CCATGTACTATTGTACAGTGTGGTATCTGGAG | |
| | ACCCCCGACGATGGCTTCGATATCTGGGGCC | |
| | AGGGCACCATGGTGACAGTGAGCTCC | |
| SEQ ID NO: 37 | CAGGTGCAGCTGGTGGAGAGCGGAGGAGG | MGU10v2 VH |
| | AGTGGTGCAGCCAGGCCGGTCCCTGAGACT | |
| | GTCTTGCGCCGCCAGCGGCTTCGCCTTTTCC | |
| | AACTACGGCATGAATTGGGTGCGGCAGGCA | |
| | CCTGGCAAGGGACTGGAGTGGGTGGCCGT | |
| | GATCTGGCACGACGGATCCCTGAAGTACTAT | |
| | ACACAGTCTGTGAAGGGCAGGTTCACCATCT | |
| | CTCGCGACAACGCCAAGAATACACTGTTTCT | |
| | GCAGATGGATTCTCTGAGCGCCGACGATACC | |
| | GCCATGTACTATTGTACAGTGTGGTATCTGG | |
| | AGACCCCCGACGAGGGCTTCGATATCTGGG | |
| | GCAGGGGCACCATGGTGACAGTGAGCTCC | |
| SEQ ID NO: 38 | CAGCTGGTGCTGACACAGCCACCTAGCGCCT | MGU10v3 VL |
| | CCGCCTCTCTGGGCGTGAGCGTGACCCTGA | |
| | CATGCACCCTGTCCCACGGCCACACCTCTAA | |
| | GGCAATCGCATGGCACCAGCAGCAGCCAGG | |
| | CAAGGGACCACGGTACCTGATGAAGGTGAA | |
| | CAGCGACGGATCCCACACAAAGGGAGCAGC | |
| | AGTGCCAGATCGGTTCAGCGGCTCCACATCT | |
| | GGCGCCGAGAGACACTTTACCATCTCTAATC | |
| | TGCAGAGCGACGATGAGGCCGACTACTATT | |
| | GTCAGGCCTGGGAGTCCGGAATCTGGGTGT | |
| | TCGGAGGAGGAACAAAGCTGACCGTGCTG | |
| SEQ ID NO: 39 | TACATCGTGATGACCCAGTCCCCCCTGTCTCT | MGH2v1 VL |
| | GCCTGTGACACTGGGCCAGCCTGCCTCTATC | |
| | AGCTGCCGGAGCTCCCAGAGCCTGGTGTAC | |
| | TCCGACGGCAACACCTACCTGAATTGGTATC | |
| | AGCAGAGGCCAGGACAGTCCCCACGGAGAC | |
| | TGATCTATAAGGTGTCTAACAGGGACAGCGG | |
| | AGTGCCAGATCGCTTCTCCGGATCTGGAAGC | |
| | GGAACCGACTTTACACTGAAGATCTCTCGGG | |
| | TGGAGGCCGAGGATGTGGGCGTGTACTATT | |
| | GTATGCAGGGCACCCACTTCTGGACATTTGG | |
| | CCAGGGCACAAAGGTGGAGATCAAG | |
| SEQ ID NO: 40 | CAGGTGCAGCTGGTGGAGAGCGGCGGCGG | MGU10 VHv1 |
| | CGTGGTGCAGCCCGGCAGAAGCCTGAGACT | |
| | GAGCTGCGCCGCCAGCGGCTTCGCCTTCAG | |
| | CAACTACGGCATGAACTGGGTGAGACAGGC | |
| | CCCCGGCAAGGGCCTGGAGTGGGTGGCCG | |
| | TGATCTGGCACGACGGCAGCCTGAAGTACTA | |
| | CACCCAGAGCGTGAAGGGCAGATTCACCAT | |
| | CAGCAGAGACAACGCCAAGAACACCCTGTTC | |
| | CTGCAGATGGACAGCCTGAGCGCCGACGAC | |
| | ACCGCCATGTACTACTGCACCGTGTGGTACC | |
| | TGGAGACCCCCGACGACGGCTTCGACATCT | |
| | GGGGCAGAGGCACCATGGTGACCGTGAGC | |
| | AGC | |
| SEQ ID NO: 41 | CAGCTTCAGTTAGTCGAGAGTGGGGGCGGG | MGU10 VHv2 |
| | GTTGTGCAACCAGGGAGATCGTTGAGGCTC | |
| | AGCTGCGCAGCCAGTGGCTTCGCCTTCAGTA | |
| | ATTACGGCATGAACTGGGTTAGGCAGGCTCC | |
| | TGGCAAAGGTTTGGAGTGGGTAGCAGTGAT | |
| | TTGGCATGACGGCTCTTTGAAATATTACACAC | |
| | AGAGTGTGAAAGGAAGATTCACAATCAGCA | |
| | GAGACAACGCCAAGAATACTCTGTTCCTGCA | |
| | GATGGATTCCCTGTCAGCCGACGACACGGC | |
| | CATGTATTACTGTACCGTGTGGTATCTCGAGA | |
| | CACCCGATGATGGCTTCGACATCTGGGGGA | |
| | GAGGCACCATGGTTACCGTGAGCAGC | |
| SEQ ID NO: 42 | CAGGTCCAGCTGGTCGAGTCAGGCGGGGG | MGU10 VHv3 |
| | CGTTGTCCAACCGGGACGCTCTTTGCGATTA | |
| | TCTTGCGCAGCGTCCGGCTTTGCGTTCAGTA | |
| | ATTATGGCATGAACTGGGTCCGACAAGCTCC | |
| | CGGAAAAGGGCTGGAATGGGTTGCGGTGAT | |
| | TTGGCATGACGGAAGCTTGAAGTACTATACG | |
| | CAGTCAGTGAAAGGAAGGTTCACAATTTCAC | |
| | GGGATAATGCGAAGAACACTCTATTCCTACA | |
| | GATGGACTCACTTTCCGCTGACGACACCGCC | |
| | ATGTATTACTGCACCGTTTGGTACTTGGAAAC | |
| | GCCGGACGACGGGTTTGATATCTGGGGCAG | |
| | AGGGACAATGGTTACCGTTTCCTCA | |
| SEQ ID NO: 43 | CAGGTGCAGCTGGTGGAGAGCGGCGGCGG | MGU10 heavy chain |
| | CGTGGTGCAGCCCGGCAGAAGCCTGAGACT | variant 1 |
| | GAGCTGCGCCGCCAGCGGCTTCGCCTTCAG | |
| | CAACTACGGCATGAACTGGGTGAGACAGGC | |
| | CCCCGGCAAGGGCCTGGAGTGGGTGGCCG | |
| | TGATCTGGCACGACGGCAGCCTGAAGTACTA | |
| | CACCCAGAGCGTGAAGGGCAGATTCACCAT | |
| | CAGCAGAGACAACGCCAAGAACACCCTGTTC | |
| | CTGCAGATGGACAGCCTGAGCGCCGACGAC | |
| | ACCGCCATGTACTACTGCACCGTGTGGTACC | |
| | TGGAGACCCCCGACGACGGCTTCGACATCT | |
| | GGGGCAGAGGCACCATGGTGACCGTGAGC | |
| | AGCGCCAGCACCAAGGGCCCCAGCGTGTTC | |
| | CCCCTGGCCCCCAGCAGCAAGAGCACCAGC | |
| | GGCGGCACCGCCGCCCTGGGCTGCCTGGTG | |
| | AAGGACTACTTCCCCGAGCCCGTGACCGTGA | |
| | GCTGGAACAGCGGCGCCCTGACCAGCGGC | |
| | GTGCACACCTTCCCCGCCGTGCTGCAGAGCA | |
| | GCGGCCTGTACAGCCTGAGCAGCGTGGTGA | |
| | CCGTGCCCAGCAGCAGCCTGGGCACCCAGA | |
| | CCTACATCTGCAACGTGAACCACAAGCCCAG | |
| | CAACACCAAGGTGGACAAGAAGGTGGAGCC | |
| | CAAGAGCTGCGACAAGACCCACACCTGCCC | |
| | CCCCTGCCCCGCCCCCGAGCTGCTGGGCGG | |
| | CCCCAGCGTGTTCCTGTTCCCCCCCAAGCCC | |
| | AAGGACACCCTGATGATCAGCAGAACCCCC | |
| | GAGGTGACCTGCGTGGTGGTGGACGTGAGC | |
| | CACGAGGACCCCGAGGTGAAGTTCAACTGG | |
| | TACGTGGACGGCGTGGAGGTGCACAACGCC | |
| | AAGACCAAGCCCAGAGAGGAGCAGTACAAC | |
| | AGCACCTACAGAGTGGTGAGCGTGCTGACC | |
| | GTGCTGCACCAGGACTGGCTGAACGGCAAG | |
| | GAGTACAAGTGCAAGGTGAGCAACAAGGCC | |
| | CTGCCCGCCCCCATCGAGAAGACCATCAGCA | |
| | AGGCCAAGGGCCAGCCCAGAGAGCCCCAG | |
| | GTGTACACCCTGCCCCCCAGCAGAGACGAG | |
| | CTGACCAAGAACCAGGTGAGCCTGACCTGC | |
| | CTGGTGAAGGGCTTCTACCCCAGCGACATCG | |
| | CCGTGGAGTGGGAGAGCAACGGCCAGCCC | |
| | GAGAACAACTACAAGACCACCCCCCCCGTGC | |
| | TGGACAGCGACGGCAGCTTCTTCCTGTACAG | |
| | CAAGCTGACCGTGGACAAGAGCAGATGGCA | |
| | GCAGGGCAACGTGTTCAGCTGCAGCGTGCT | |
| | GCACGAGGCCCTGCACAGCCACTACACCCA | |
| | GAAGAGCCTGAGCCTGAGCCCCGGCAAG | |
| SEQ ID NO: 44 | CAGGTTCAGTTAGTCGAGAGTGGGGGCGGG | MGU10 heavy chain |
| | GTTGTGCAACCAGGGAGATCGTTGAGGCTC | variant 2 |
| | AGCTGCGCAGCCAGTGGCTTCGCCTTCAGTA | |
| | ATTACGGCATGAACTGGGTTAGGCAGGCTCC | |
| | TGGCAAAGGTTTGGAGTGGGTAGCAGTGAT | |
| | TTGGCATGACGGCTCTTTGAAATATTACACAC | |
| | AGAGTGTGAAAGGAAGATTCACAATCAGCA | |
| | GAGACAACGCCAAGAATACTCTGTTCCTGCA | |
| | GATGGATTCCCTGTCAGCCGACGACACGGC | |
| | CATGTATTACTGTACCGTGTGGTATCTCGAGA | |
| | CACCCGATGATGGCTTCGACATCTGGGGGA | |
| | GAGGCACCATGGTTACCGTGAGCAGCGCTT | |
| | CCACCAAAGGCCCATCCGTCTTCCCACTGGC | |
| | CCCCTCCTCAAAAAGTACCTCAGGAGGGACC | |
| | GCCGCCTTGGGCTGCCTTGTAAAAGACTACT | |
| | TCCCTGAGCCCGTTACTGTCTCTTGGAACTCC | |
| | GGCGCTCTGACCTCCGGCGTTCATACGTTTC | |
| | CTGCCGTGCTTCAGTCCAGTGGCTTGTATTCC | |
| | CTGAGCTCTGTAGTCACCGTGCCGAGCAGTA | |
| | GCCTCGGCACACAGACGTACATATGTAACGT | |
| | GAATCACAAGCCATCTAACACTAAAGTCGAT | |
| | AAAAAAGTAGAGCCTAAAAGCTGTGATAAAA | |
| | CCCACACATGTCCGCCATGTCCCGCGCCCGA | |
| | ACTGTTGGGCGGGCCCAGTGTGTTCCTATTC | |
| | CCACCAAAACCGAAAGACACCTTGATGATCA | |
| | GTCGCACACCTGAGGTAACCTGCGTGGTCGT | |
| | CGACGTCTCCCACGAAGACCCCGAGGTCAA | |
| | GTTTAACTGGTATGTTGATGGCGTCGAGGTA | |
| | CACAATGCAAAAACCAAACCAAGGGAAGAA | |
| | CAATATAATAGTACATATCGCGTGGTGAGTG | |
| | TCCTCACCGTGCTCCACCAGGACTGGCTAAA | |
| | TGGGAAGGAGTATAAATGCAAGGTGAGCAA | |
| | CAAGGCACTCCCGGCCCCAATTGAGAAAACC | |
| | ATTTCCAAAGCCAAGGGCCAACCACGAGAAC | |
| | CACAGGTCTACACCCTCCCACCTTCACGCGA | |
| | CGAGTTGACAAAGAATCAAGTGTCTCTCACC | |
| | TGTCTTGTGAAGGGGTTTTATCCCAGTGATAT | |
| | CGCGGTGGAATGGGAGAGCAATGGACAACC | |
| | AGAGAACAACTATAAGACCACCCCGCCTGTC | |
| | CTGGATTCCGACGGATCTTTCTTCCTTTATTCA | |
| | AAGTTGACCGTGGACAAGTCCCGGTGGCAG | |
| | CAAGGGAATGTGTTCAGCTGCTCGGTGCTCC | |
| | ATGAGGCCCTTCACAGCCACTATACGCAGAA | |
| | GAGCTTATCTCTGAGCCCTGGGAAG | |
| SEQ ID NO: 45 | CAGGTCCAGCTGGTCGAGTCAGGCCGGGG | MGU10 heavy chain |
| | CGTTGTCCAACCGGGACGCTCTTTGCGATTA | variant 3 |
| | TCTTGCGCAGCGTCCGGCTTTGCGTTCAGTA | |
| | ATTATGGCATGAACTGGGTCCGACAAGCTCC | |
| | CGGAAAAGGGCTGGAATGGGTTGCGGTGAT | |
| | TTGGCATGACGGAAGCTTGAAGTACTATACG | |
| | CAGTCAGTGAAAGGAAGGTTCACAATTTCAC | |
| | GGGATAATGCGAAGAACACTCTATTCCTACA | |
| | GATGGACTCACTTTCCGCTGACGACACCGCC | |
| | ATGTATTACTGCACCGTTTGGTACTTGGAAAC | |
| | GCCGGACGACGGGTTTGATATCTGGGGCAG | |
| | AGGGACAATGGTTACCGTTTCCTCAGCCAGT | |
| | ACGAAGGGGCCCTCAGTATTTCCGCTAGCGC | |
| | CGAGCTCAAAGTCGACATCTGGGGGCACAG | |
| | CAGCACTGGGATGTCTGGTCAAAGATTACTT | |
| | CCCCGAGCCTGTAACCGTTAGTTGGAATAGT | |
| | GGTGCCTTAACGAGTGGGGTTCATACATTTC | |
| | CAGCGGTACTCCAGTCCTCAGGGCTCTACTC | |
| | CTTATCAAGCGTCGTTACAGTCCCAAGTTCAT | |
| | CGCTAGGTACTCAAACTTACATCTGCAATGTT | |
| | AACCATAAGCCCAGCAATACCAAAGTCGACA | |
| | AAAAAGTCGAACCGAAGTCCTGCGACAAGA | |
| | CGCACACGTGTCCACCTTGTCCTGCCCCGGA | |
| | GTTATTGGGCGGCCCGTCGGTGTTCTTGTTT | |
| | CCTCCCAAACCGAAGGATACCCTAATGATTTC | |
| | GAGGACGCCAGAAGTAACATGTGTTGTGGT | |
| | CGATGTATCTCATGAAGACCCAGAGGTTAAG | |
| | TTCAACTGGTATGTCGATGGCGTCGAAGTAC | |
| | ACAACGCAAAGACCAAACCCAGGGAAGAAC | |
| | AGTACAATAGTACTTATAGGGTTGTATCAGTA | |
| | CTTACGGTCCTGCATCAGGACTGGCTTAACG | |
| | GTAAAGAGTACAAATGTAAGGTGTCTAATAA | |
| | GGCACTGCCCGCGCCAATTGAAAAAACCATC | |
| | TCGAAAGCTAAGGGCCAGCCCAGAGAACCT | |
| | CAAGTGTACACGCTTCCGCCGAGTCGCGAC | |
| | GAACTGACTAAGAACCAGGTTTCTCTGACTT | |
| | GCCTAGTTAAAGGTTTCTACCCGTCGGACAT | |
| | AGCAGTCGAATGGGAAAGCAACGGCCAGCC | |
| | GGAGAACAACTACAAGACCACGCCTCCGGT | |
| | GCTCGATTCGGATGGGTCTTTCTTTTTATATTC | |
| | GAAATTAACCGTGGATAAAAGTCGGTGGCAA | |
| | CAAGGTAATGTTTTCAGTTGTTCTGTCCTTCA | |
| | CGAAGCCCTACATTCGCACTACACGCAAAAG | |
| | AGTTTAAGTTTGTCACCGGGGAAG | |
| SEQ ID NO: 46 | CAGCTGGTGCTGACCCAGCCCCCCAGCGCC | MGU10 VLv1 |
| | AGCGCCAGCCTGGGCGTGAGCGTGACCCTG | |
| | ACCTGCACCCTGAGCCACGGCCACACCAGC | |
| | AAGGCCATCGCCTGGCACCAGCAGCAGCCC | |
| | GGCAAGGGCCCCAGATACCTGATGAAGGTG | |
| | AACAGCGACGGCAGCCACACCAAGGGCGCC | |
| | GCCGTGCCCGACAGATTCAGCGGCAGCACC | |
| | AGCGGCGCCGAGAGACACTTCACCATCAGC | |
| | AACCTGCAGAGCGACGACGAGGCCGACTAC | |
| | TACTGCCAGGCCTGGGACAGCGGCATCTGG | |
| | GTGTTCGGCGGCGGCACCAAGCTGACCGTG | |
| | CTG | |
| SEQ ID NO: 47 | CAGTTGGTGTTGACCCAACCACCGTCTGCCT | MGU10 VLv2 |
| | CTGCAAGTCTGGGGGTGTCTGTGACACTTAC | |
| | TTGCACGCTGTCTCACGGGCACACAAGCAAG | |
| | GCCATTGCTTGGCACCAACAGCAGCCTGGCA | |
| | AGGGACCTCGGTACTTGATGAAGGTCAACTC | |
| | CGACGGCAGTCATACCAAAGGTGCAGCAGT | |
| | GCCGGATAGATTTTCCGGCTCCACAAGTGGC | |
| | GCGGAGCGCCACTTTACCATCTCCAACCTTC | |
| | AGAGCGATGACGAAGCTGATTATTATTGTCA | |
| | GGCCTGGGACTCAGGCATATGGGTATTCGG | |
| | GGGGGGGACCAAGCTCACCGTGTTA | |
| SEQ ID NO: 48 | CAACTCGTTCTCACCCAACCGCCTTCAGCCA | MGU10 VLv3 |
| | GCGCCAGTCTCGGAGTATCGGTGACCCTTAC | |
| | GTGCACACTCTCACACGGCCACACGTCGAAG | |
| | GCTATAGCCTGGCATCAGCAACAACCGGGC | |
| | AAAGGACCGCGTTATCTAATGAAGGTCAATT | |
| | CCGACGGATCTCATACAAAAGGCGCCGCCG | |
| | TACCTGACCGCTTTAGCGGGAGTACGTCCGG | |
| | GGCAGAGCGTCATTTCACGATAAGTAATCTC | |
| | CAGTCGGATGACGAGGCTGACTATTATTGTC | |
| | AGGCCTGGGACTCAGGTATTTGGGTATTTGG | |
| | AGGGGGGACCAAGTTGACGGTCTTA | |
| SEQ ID NO: 49 | CAGCTGGTGCTGACCCAGCCCCCCAGCCCC | MGU10 light chain |
| | AGCGCCAGCCTGGGCGTGAGCGTGACCCTG | variant 1 |
| | ACCTGCACCCTGAGCCACGGCCACACCAGC | |
| | AAGGCCATCGCCTGGCACCAGCAGCAGCCC | |
| | GGCAAGGGCCCCAGATACCTGATGAAGGTG | |
| | AACAGCGACGGCAGCCACACCAAGGGCGCC | |
| | GCCGTGCCCGACAGATTCAGCGGCAGCACC | |
| | AGCGGCGCCGAGAGACACTTCACCATCAGC | |
| | AACCTGCAGAGCGACGACGAGGCCGACTAC | |
| | TACTGCCAGGCCTGGGACAGCGGCATCTGG | |
| | GTGTTCGGCGGCGGCACCAAGCTGACCGTG | |
| | CTGGGCCAGCCCAAGGCCGCCCCCAGCGTG | |
| | ACCCTGTTCCCCCCCAGCAGCGAGGAGCTG | |
| | CAGGCCAACAAGGCCACCCTGGTGTGCCTG | |
| | ATCAGCGACTTCTACCCCGGCGCCGTGACCG | |
| | TGGCCTGGAAGGCCGACAGCAGCCCCGTGA | |
| | AGGCCGGCGTGGAGACCACCACCCCCAGCA | |
| | AGCAGAGCAACAACAAGTACGCCGCCAGCA | |
| | GCTACCTGAGCCTGACCCCCGAGCAGTGGA | |
| | AGAGCCACAGAAGCTACAGCTGCCAGGTGA | |
| | CCCACGAGGGCAGCACCGTGGAGAAGACC | |
| | GTGGCCCCCACCGAGTGCAGC | |
| SEQ ID NO: 50 | CAGTTGGTGTTGACCCAACCACCGTCTGCCT | MGU10 light chain |
| | CTGCAAGTCTGGGGGTGTCTGTGACACTTAC | variant 2 |
| | TTGCACGCTGTCTCACGGGCACACAAGCAAG | |
| | GCCATTGCTTGGCACCAACAGCAGCCTGGCA | |
| | AGGGACCTCGGTACTTGATGAAGGTCAACTC | |
| | CGACGGCAGTCATACCAAAGGTGCAGCAGT | |
| | GCCGGATAGATTTTCCGGCTCCACAAGTGGC | |
| | GCGGAGCGCCACTTTACCATCTCCAACCTTC | |
| | AGAGCGATGACGAAGCTGATTATTATTGTCA | |
| | GGCCTGGGACTCAGGCATATGGGTATTCGG | |
| | GGGGGGGACCAAGCTCACCGTGTTAGGGCA | |
| | ACCGAAAGCGGCACCCAGTGTGACCCTGTTT | |
| | CCCCCCAGCAGTGAGGAACTCCAGGCAAAT | |
| | AAGGCCACTTTGGTCTGTCTGATTAGTGATTT | |
| | TTATCCCGGGGCAGTCACCGTGGCTTGGAAA | |
| | GCGGACTCTTCTCCCGTAAAAGCCGGAGTCG | |
| | AGACCACTACACCGTCTAAGCAGAGTAATAA | |
| | CAAATATGCTGCTAGCTCTTACCTGTCCCTGA | |
| | CACCAGAACAGTGGAAGTCCCATAGGAGTTA | |
| | TAGCTGCCAGGTCACACACGAGGGGAGCAC | |
| | CGTGGAGAAGACAGTTGCACCCACTGAGTG | |
| | CTCC | |
| SEQ ID NO: 51 | CAACTCCTTCTCACCCAACCGCCTTCAGCCA | MGU10 light chain |
| | GCGCCAGTCTCGGAGTATCGGTGACCCTTAC | variant 3 |
| | GTGCACACTCTCACACGGCCACACGTCGAAG | |
| | GCTATAGCCTGGCATCAGCAACAACCGGGC | |
| | AAAGGACCGCGTTATCTAATGAAGGTCAATT | |
| | CCGACGGATCTCATACAAAAGGCGCCGCCG | |
| | TACCTGACCGCTTTAGCGGGAGTACGTCCGG | |
| | GGCAGAGCGTCATTTCACGATAAGTAATCTC | |
| | CAGTCGGATGACGAGGCTGACTATTATTGTC | |
| | AGGCCTGGGACTCAGGTATTTGGGTATTTGG | |
| | AGGGGGGACCAAGTTGACGGTCTTAGGCCA | |
| | GCCTAAGGCGGCGCCGTCTGTTACTTTATTC | |
| | CCTCCTTCTTCGGAGGAGCTTCAGGCCAACA | |
| | AGGCCACCCTTGTATGTCTTATATCCGACTTT | |
| | TATCCTGGAGCCGTTACTGTTGCGTGGAAGG | |
| | CCGACTCGTCGCCTGTCAAGGCCGGGGTCG | |
| | AGACTACGACCCCTTCAAAGCAAAGTAACAA | |
| | TAAGTACGCTGCAAGCTCTTATCTGTCACTAA | |
| | CGCCTGAGCAGTGGAAGTCGCACAGATCAT | |
| | ATAGCTGCCAGGTTACCCATGAAGGGAGCA | |
| | CTGTGGAAAAAACCGTTGCACCAACTGAATG | |
| | CAGC | |
| SEQ ID NO: 52 | CAGGTGCAGCTGGTGGAGAGCGGCGGCGG | MGH2 VHv1 |
| | CGTGGTGCAGCCCGGCGGCAGCCTGAGACT | |
| | GAGCTGCACCGCCAGCGGCTTCAGCTTCAG | |
| | CAGCTACGCCATGCACTGGGTGAGACAGGC | |
| | CCCCGGCAAGGGCCTGGAGTGGGTGGCCTA | |
| | CACCAGATACGACGGCAGCAACAAGTTCTAC | |
| | CTGGACAGCGTGCAGGGCAGATTCACCATC | |
| | AGCAGAGACAACAGCAAGAACACCCTGTAC | |
| | CTGGAGATGGACAGCCTGAGACTGGAGGAC | |
| | ACCGCCGTGTACTTCTGCGCCAAGGTGGGC | |
| | GACGGCACCGTGGCCGGCACCATCGACTAC | |
| | TGGGGCCAGGGCACCCTGGTGACCGTGAGC | |
| | AGC | |
| SEQ ID NO: 53 | CAGGTGCAGCTGGTGGAAAGCGGGGGGGG | MGH2 VHv2 |
| | AGTCGTGCAGCCTGGCGGCTCTTTGCGGCT | |
| | GTCTTGTACGGCTTCTGGATTCTCATTCTCTTC | |
| | CTACGCCATGCACTGGGTCCGCCAGGCGCC | |
| | CGGGAAGGGGCTGGAATGGGTTGCCTACAC | |
| | AAGGTATGATGGTTCAAACAAGTTCTACTTAG | |
| | ATTCAGTGCAGGGTAGATTCACTATAAGCCG | |
| | GGACAATAGTAAGAACACTCTCTACCTAGAA | |
| | ATGGACTCTCTCAGACTGGAAGATACCGCTG | |
| | TGTACTTCTGTGCTAAGGTTGGGGACGGCAC | |
| | CGTTGCCGGCACAATCGACTATTGGGGACAA | |
| | GGGACCCTCGTCACAGTCAGCTCG | |
| SEQ ID NO: 54 | CAAGTCCAGCTTGTCGAGTCGGGGGGAGGG | MGH2 VHv3 |
| | GTTGTCCAGCCTGGTGGTAGCTTACGCCTGA | |
| | GTTGTACAGCATCGGGGTTTAGCTTCTCTTCC | |
| | TATGCGATGCACTGGGTGAGACAGGCTCCC | |
| | GGAAAGGGCTTAGAGTGGGTGGCCTACACT | |
| | CGGTATGACGGTTCGAATAAGTTTTACTTAGA | |
| | CAGCGTTCAGGGTAGGTTCACCATCTCACGT | |
| | GATAATAGTAAGAATACATTATATCTTGAGAT | |
| | GGACAGCCTTCGGTTGGAGGATACTGCCGT | |
| | CTACTTTTGTGCTAAGGTAGGCGATGGTACG | |
| | GTAGCAGGCACGATAGATTACTGGGGCCAA | |
| | GGAACGTTGGTCACTGTCTCTTCA | |
| SEQ ID NO: 55 | CAGGTGCAGCTGGTGGAGAGCGGCGGCGG | MGH2 heavy chain |
| | CGTGGTGCAGCCCGGCGGCAGCCTGAGACT | variant 1 |
| | GAGCTGCACCGCCAGCGGCTTCAGCTTCAG | |
| | CAGCTACGCCATGCACTGGGTGAGACAGGC | |
| | CCCCGGCAAGGGCCTGGAGTGGGTGGCCTA | |
| | CACCAGATACGACGGCAGCAACAAGTTCTAC | |
| | CTGGACAGCGTGCAGGGCAGATTCACCATC | |
| | AGCAGAGACAACAGCAAGAACACCCTGTAC | |
| | CTGGAGATGGACAGCCTGAGACTGGAGGAC | |
| | ACCGCCGTGTACTTCTGCGCCAAGGTGGGC | |
| | GACGGCACCGTGGCCGGCACCATCGACTAC | |
| | TGGGGCCAGGGCACCCTGGTGACCGTGAGC | |
| | AGCGCCAGCACCAAGGGCCCCAGCGTGTTC | |
| | CCCCTGGCCCCCAGCAGCAAGAGCACCAGC | |
| | GGCGGCACCGCCGCCCTGGGCTGCCTGGTG | |
| | AAGGACTACTTCCCCGAGCCCGTGACCGTGA | |
| | GCTGGAACAGCGGCGCCCTGACCAGCGGC | |
| | GTGCACACCTTCCCCGCCGTGCTGCAGAGCA | |
| | GCGGCCTGTACAGCCTGAGCAGCGTGGTGA | |
| | CCGTGCCCAGCAGCAGCCTGGGCACCCAGA | |
| | CCTACATCTGCAACGTGAACCACAAGCCCAG | |
| | CAACACCAAGGTGGACAAGAAGGTGGAGCC | |
| | CAAGAGCTGCGACAAGACCCACACCTGCCC | |
| | CCCCTGCCCCGCCCCCGAGCTGCTGGGCGG | |
| | CCCCAGCGTGTTCCTGTTCCCCCCCAAGCCC | |
| | AAGGACACCCTGATGATCAGCAGAACCCCC | |
| | GAGGTGACCTGCGTGGTGGTGGACGTGAGC | |
| | CACGAGGACCCCGAGGTGAAGTTCAACTGG | |
| | TACGTGGACGGCGTGGAGGTGCACAACGCC | |
| | AAGACCAAGCCCAGAGAGGAGCAGTACAAC | |
| | AGCACCTACAGAGTGGTGAGCGTGCTGACC | |
| | GTGCTGCACCAGGACTGGCTGAACGGCAAG | |
| | GAGTACAAGTGCAAGGTGAGCAACAAGGCC | |
| | CTGCCCGCCCCCATCGAGAAGACCATCAGCA | |
| | AGGCCAAGGGCCAGCCCAGAGAGCCCCAG | |
| | GTGTACACCCTGCCCCCCAGCAGAGACGAG | |
| | CTGACCAAGAACCAGGTGAGCCTGACCTGC | |
| | CTGGTGAAGGGCTTCTACCCCAGCGACATCG | |
| | CCGTGGAGTGGGAGAGCAACGGCCAGCCC | |
| | GAGAACAACTACAAGACCACCCCCCCCGTGC | |
| | TGGACAGCGACGGCAGCTTCTTCCTGTACAG | |
| | CAAGCTGACCGTGGACAAGAGCAGATGGCA | |
| | GCAGGGCAACGTGTTCAGCTGCAGCGTGCT | |
| | GCACGAGGCCCTGCACAGCCACTACACCCA | |
| | GAAGAGCCTGAGCCTGAGCCCCGGCAAG | |
| SEQ ID NO: 56 | CAGGTGCAGCTGGTGGAAAGCGGGGGGGG | MGH2 heavy chain |
| | AGTCGTGCAGCCTGGCGGCTCTTTGCGGCT | variant 2 |
| | GTCTTGTACGGCTTCTGGATTCTCATTCTCTTC | |
| | CTACGCCATGCACTGGGTCCGCCAGGCGCC | |
| | CGGGAAGGGGCTGGAATGGGTTGCCTACAC | |
| | AAGGTATGATGGTTCAAACAAGTTCTACTTAG | |
| | ATTCAGTGCAGGGTAGATTCACTATAAGCCG | |
| | GGACAATAGTAAGAACACTCTCTACCTAGAA | |
| | ATGGACTCTCTCAGACTGGAAGATACCGCTG | |
| | TGTACTTCTGTGCTAAGGTTGGGGACGGCAC | |
| | CGTTGCCGGCACAATCGACTATTGGGGACAA | |
| | GGGACCCTCGTCACAGTCAGCTCGGCCAGT | |
| | ACCAAGGGCCCCAGTGTGTTCCCTTTGGCCC | |
| | CTTCTAGCAAATCAACGTCAGGGGGGACAG | |
| | CCGCCCTTGGGTGTCTTGTGAAAGATTATTTT | |
| | CCTGAGCCGGTGACCGTTTCCTGGAATAGTG | |
| | GAGCACTGACAAGCGGCGTACATACCTTCCC | |
| | AGCAGTGCTCCAATCAAGTGGGCTATACAGT | |
| | CTGAGTAGCGTGGTCACCGTGCCATCTTCCT | |
| | CTCTGGGAACTCAAACCTATATTTGCAACGTT | |
| | AATCACAAACCTTCTAATACGAAAGTCGATAA | |
| | GAAGGTAGAACCGAAGTCCTGCGACAAAAC | |
| | CCACACATGCCCTCCGTGCCCAGCCCCTGAG | |
| | CTACTGGGCGGCCCCTCTGTGTTTTTGTTTCC | |
| | CCCAAAACCTAAGGATACCTTAATGATCTCAA | |
| | GAACACCCGAGGTGACCTGCGTCGTGGTAG | |
| | ATGTTTCTCACGAGGACCCTGAGGTTAAATTT | |
| | AATTGGTACGTGGACGGCGTGGAGGTCCAT | |
| | AACGCCAAGACTAAACCAAGAGAAGAGCAG | |
| | TACAACTCCACATACAGGGTTGTGTCTGTGCT | |
| | GACGGTCTTACACCAAGACTGGCTTAACGGC | |
| | AAAGAGTATAAATGCAAGGTGAGTAATAAAG | |
| | CGCTTCCTGCCCCAATCGAAAAGACCATCAG | |
| | TAAAGCCAAAGGACAGCCCAGGGAGCCTCA | |
| | AGTCTACACGTTACCTCCCTCAAGGGATGAG | |
| | CTCACCAAAAACCAGGTGTCTCTGACCTGCC | |
| | TGGTTAAGGGCTTTTATCCTTCAGACATCGCT | |
| | GTTGAGTGGGAATCAAATGGGCAGCCAGAA | |
| | AATAACTATAAAACTACCCCTCCTGTGCTGGA | |
| | CAGTGACGGTTCGTTCTTCCTCTATAGTAAGT | |
| | TGACCGTGGATAAAAGCCGATGGCAGCAGG | |
| | GAAATGTGTTCAGCTGCTCTGTACTACATGA | |
| | GGCCCTCCACAGTCACTATACGCAGAAGTCA | |
| | CTGAGTCTAAGTCCAGGGAAG | |
| SEQ ID NO: 57 | CAAGTCCAGCTTGTCGAGTCGGGGGGAGGG | MGH2 heavy chain |
| | GTTGTCCAGCCTGGTGGTAGCTTACGCCTGA | variant 3 |
| | GTTGTACAGCATCGGGGTTTAGCTTCTCTTCC | |
| | TATGCGATGCACTGGGTGAGACAGGCTCCC | |
| | GGAAAGGGCTTAGAGTGGGTGGCCTACACT | |
| | CGGTATGACGGTTCGAATAAGTTTTACTTAGA | |
| | CAGCGTTCAGGGTAGGTTCACCATCTCACGT | |
| | GATAATAGTAAGAATACATTATATCTTGAGAT | |
| | GGACAGCCTTCGGTTGGAGGATACTGCCGT | |
| | CTACTTTTGTGCTAAGGTAGGCGATGGTACG | |
| | GTAGCAGGCACGATAGATTACTGGGGCCAA | |
| | GGAACGTTGGTCACTGTCTCTTCAGCATCTAC | |
| | CAAGGGTCCATCTGTCTTCCCACTGGCCCCA | |
| | TCCTCCAAAAGCACGAGCGGAGGCACCGCT | |
| | GCTCTAGGGTGTCTCGTCAAGGACTACTTTC | |
| | CCGAGCCAGTGACAGTTAGTTGGAATTCCGG | |
| | TGCACTTACGTCGGGGGTTCACACATTCCCA | |
| | GCAGTGCTGCAGTCGAGCGGCCTCTACAGC | |
| | TTGTCCTCAGTCGTAACTGTTCCATCCAGTTC | |
| | GCTCGGGACTCAGACTTACATCTGCAATGTA | |
| | AACCACAAACCATCCAATACGAAGGTGGATA | |
| | AAAAGGTTGAGCCTAAGTCATGCGACAAGAC | |
| | ACATACGTGCCCACCATGTCCCGCGCCAGAG | |
| | TTGCTTGGCGGACCCAGCGTCTTTCTGTTCCC | |
| | ACCTAAACCCAAGGACACGTTGATGATTAGC | |
| | AGGACCCCCGAAGTTACTTGTGTCGTGGTGG | |
| | ATGTAAGCCATGAAGACCCAGAGGTGAAATT | |
| | TAACTGGTATGTGGATGGAGTTGAAGTCCAT | |
| | AATGCGAAGACAAAACCTCGAGAGGAACAA | |
| | TATAACTCCACGTATCGAGTCGTGTCCGTACT | |
| | CACAGTGTTACATCAAGATTGGTTAAATGGTA | |
| | AAGAGTACAAATGCAAGGTTTCGAATAAAGC | |
| | ACTGCCAGCGCCGATCGAAAAGACTATCTCA | |
| | AAGGCAAAAGGCCAGCCCCGGGAGCCTCAA | |
| | GTATATACGCTGCCGCCATCGCGCGACGAGT | |
| | TAACAAAAAACCAAGTATCGTTGACGTGTTTG | |
| | GTGAAAGGTTTTTACCCTTCGGATATAGCCGT | |
| | GGAGTGGGAATCCAACGGTCAACCAGAGAA | |
| | CAACTATAAGACAACCCCACCAGTCTTAGATA | |
| | GTGATGGCTCTTTCTTCCTCTATAGTAAACTA | |
| | ACGGTCGATAAATCTCGCTGGCAGCAGGGC | |
| | AACGTCTTTTCGTGTTCGGTTTTACATGAAGC | |
| | TCTACATAGTCACTATACCCAGAAGAGTCTAT | |
| | CTCTAAGCCCCGGCAAG | |
| SEQ ID NO: 58 | TACATCGTGATGACCCAGAGCCCCCTGAGCC | MGH2 VLv1 |
| | TGCCCGTGACCCTGGGCCAGCCCGCCAGCA | |
| | TCAGCTGCAGAAGCAGCCAGAGCCTGGTGT | |
| | ACAGCGACGGCAACACCTACCTGAACTGGTA | |
| | CCAGCAGAGACCCGGCCAGAGCCCCAGAAG | |
| | ACTGATCTACAAGGTGAGCAACAGAGACAG | |
| | CGGCGTGCCCGACAGATTCAGCGGCAGCGG | |
| | CAGCGGCACCGACTTCACCCTGAAGATCAGC | |
| | AGAGTGGAGGCCGAGGACGTGGGCGTGTA | |
| | CTACTGCATGCAGGGCACCCACTGGTGGAC | |
| | CTTCGGCCAGGGCACCAAGGTGGAGATCAA | |
| | G | |
| SEQ ID NO: 59 | TACATTGTTATGACCCAGAGTCCTCTTTCACT | MGH2 VLv2 |
| | GCCTGTGACCCTTGGCCAGCCTGCCTCCATC | |
| | AGCTGCCGGTCCAGCCAATCTCTCGTGTACT | |
| | CCGACGGCAATACCTACCTGAACTGGTATCA | |
| | ACAACGACCCGGCCAGTCACCCAGACGCCT | |
| | GATCTATAAGGTCAGCAATCGGGACAGCGG | |
| | CGTCCCGGATAGGTTCTCAGGTTCAGGTTCA | |
| | GGCACCGATTTCACGCTGAAAATTAGTAGAG | |
| | TTGAGGCAGAAGATGTCGGCGTGTACTACTG | |
| | TATGCAGGGTACCCATTGGTGGACCTTTGGG | |
| | CAGGGCACAAAAGTAGAGATTAAG | |
| SEQ ID NO: 60 | TACATAGTAATGACGCAGAGTCCTCTGTCCTT | MGH2 VLv3 |
| | ACCAGTTACACTGGGCCAACCTGCATCTATAT | |
| | CGTGTCGATCATCTCAGTCCCTCGTGTACTCA | |
| | GATGGAAATACGTATTTGAACTGGTATCAAC | |
| | AGCGTCCGGGACAGAGCCCTCGCCGTTTAAT | |
| | CTACAAAGTTAGTAACCGAGACAGTGGCGTT | |
| | CCTGACCGTTTCTCAGGATCAGGTTCCGGGA | |
| | CAGATTTCACCTTAAAAATAAGCAGGGTTGA | |
| | AGCTGAGGACGTGGGGGTTTATTATTGCATG | |
| | CAGGGTACCCACTGGTGGACTTTTGGACAG | |
| | GGTACGAAGGTTGAGATCAAG | |
| SEQ ID NO: 61 | TACATCGTGATGACCCAGAGCCCCCTGAGCC | MGH2 light chain |
| | TGCCCGTGACCCTGGGCCAGCCCGCCAGCA | variant 1 |
| | TCAGCTGCAGAAGCAGCCAGAGCCTGGTGT | |
| | ACAGCGACGGCAACACCTACCTGAACTGGTA | |
| | CCAGCAGAGACCCGGCCAGAGCCCCAGAAG | |
| | ACTGATCTACAAGGTGAGCAACAGAGACAGC | |
| | GGCGTGCCCGACAGATTCAGCGGCAGCGGC | |
| | AGCGGCACCGACTTCACCCTGAAGATCAGCA | |
| | GAGTGGAGGCCGAGGACGTGGGCGTGTACT | |
| | ACTGCATGCAGGGCACCCACTGGTGGACCTT | |
| | CGGCCAGGGCACCAAGGTGGAGATCAAGAG | |
| | AACCGTGGCCGCCCCCAGCGTGTTCATCTTC | |
| | CCCCCCAGCGACGAGCAGCTGAAGAGCGGC | |
| | ACCGCCAGCGTGGTGTGCCTGCTGAACAACT | |
| | TCTACCCCAGAGAGGCCAAGGTGCAGTGGA | |
| | AGGTGGACAACGCCCTGCAGAGCGGCAACA | |
| | GCCAGGAGAGCGTGACCGAGCAGGACAGC | |
| | AAGGACAGCACCTACAGCCTGAGCAGCACC | |
| | CTGACCCTGAGCAAGGCCGACTACGAGAAG | |
| | CACAAGGTGTACGCCTGCGAGGTGACCCAC | |
| | CAGGGCCTGAGCAGCCCCGTGACCAAGAGC | |
| | TTCAACAGAGGCGAGTGC | |
| SEQ ID NO: 62 | TACATTGTTATGACCCAGAGTCCTCTTTCACT | MGH2 light chain |
| | GCCTGTGACCCTTGGCCAGCCTGCCTCCATC | variant 2 |
| | AGCTGCCGGTCCAGCCAATCTCTCGTGTACT | |
| | CCGACGGCAATACCTACCTGAACTGGTATCA | |
| | ACAACGACCCGGCCAGTCACCCAGACGCCT | |
| | GATCTATAAGGTCAGCAATCGGGACAGCGG | |
| | CGTCCCGGATAGGTTCTCAGGTTCAGGTTCA | |
| | GGCACCGATTTCACGCTGAAAATTAGTAGAG | |
| | TTGAGGCAGAAGATGTCGGCGTGTACTACTG | |
| | TATGCAGGGTACCCATTGGTGGACCTTTGGG | |
| | CAGGGCACAAAAGTAGAGATTAAGCGGACT | |
| | GTGGCAGCTCCCTCAGTCTTTATATTTCCCCC | |
| | ATCCGATGAGCAGTTGAAAAGCGGGACCGC | |
| | ATCAGTTGTGTGTCTGTTGAACAAGTTTTACC | |
| | CTCGGGAGGCCAAGGTGCAGTGGAAGGTTG | |
| | ATAACGCTTTACAGTCAGGCAATTCTCAGGA | |
| | AAGTGTAACAGAACAGGATTCTAAGGACTCA | |
| | ACTTATAGCCTCTCCAGCACCCTCACATTGTC | |
| | AAAGGCCGACTATGAGAAGCACAAAGTGTAT | |
| | GCGTGTGAGGTTACACATCAGGGCCTGAGC | |
| | TCTCCGGTAACAAAGTCTTTTAACAGGGGAG | |
| | AGTGC | |
| SEQ ID NO: 63 | TACATAGTAATCACGCAGAGTCCTCTGTCCTT | MGH2 light chain |
| | ACCAGTTACACTGGGCCAACCTGCATCTATAT | variant 3 |
| | CGTGTCGATCATCTCAGTCCCTCGTGTACTCA | |
| | GATGGAAATACGTATTTGAACTGGTATCAAC | |
| | AGCGTCCGGGACAGAGCCCTCGCCGTTTAAT | |
| | CTACAAAGTTAGTAACCGAGACAGTGGCGTT | |
| | CCTGACCGTTTCTCAGGATCAGGTTCCGGGA | |
| | CAGATTTCACCTTAAAAATAAGCAGGGTTGA | |
| | AGCTGAGGACGTGGGGGTTTATTATTGCATG | |
| | CAGGGTACCCACTGGTGGACTTTTGGACAG | |
| | GGTACGAAGGTTGAGATCAAGCGGACCGTT | |
| | GCAGCCCCGTCCGTCTTTATTTTCCCCCCGTC | |
| | CGATGAACAATTGAAATCCGGTACAGCGTCA | |
| | GTAGTATGCCTCCTGAACAATTTTTATCCTCG | |
| | GGAAGCCAAGGTTCAGTGGAAAGTGGATAA | |
| | CGCACTTCAGTCCGGCAACAGTCAGGAGTCT | |
| | GTGACCGAGCAAGACTCTAAAGACTCAACCT | |
| | ACTCTCTTTCCTCGACCCTGACTCTTTCAAAG | |
| | GCGGATTATGAGAAGCACAAAGTTTATGCCT | |
| | GTGAAGTAACACATCAAGGCTTGTCGTCACC | |
| | GGTTACCAAATCGTTCAACAGAGGGGAGTG | |
| | C | |
| SEQ ID NO: 64 | GGCTTCGCCTTCAGCAACTACGGC | MGU10 CDRH1v1 |
| SEQ ID NO: 65 | GGCTTCGCCTTCAGTAATTACGGC | MGU10 CDRH1v2 |
| SEQ ID NO: 66 | GGGTTTGCGTTCAGTAATTATGGC | MGU10 CDRH1v3 |
| SEQ ID NO: 67 | ATCTGGCACGACGGCAGCCTGAAG | MGU10 CDRH2v1 |
| SEQ ID NO: 68 | ATTTGGCATGACGGCTCTTTGAAA | MGU10 CDRH2v2 |
| SEQ ID NO: 69 | ATTTGGCATGACGGAAGCTTGAAG | MGU10 CDRH2v3 |
| SEQ ID NO: 70 | ACCGTGTGGTACCTGGAGACCCCCGACGAC | MGU10 CDRH3v1 |
| | GGCTTCGACATC | |
| SEQ ID NO: 71 | ACCGTGTGGTATCTCGAGACACCCGATGATG | MGU10 CDRH3v2 |
| | GCTTCGACATC | |
| SEQ ID NO: 72 | ACCGTTTGGTACTTGGAAACGCCGGACGAC | MGU10 CDRH3v3 |
| | GGGTTTGATATC | |
| SEQ ID NO: 73 | CACGGCCACACCAGCAAGGCC | MGU10 CDRL1v1 |
| SEQ ID NO: 74 | CACGGGCACACAAGCAAGGCC | MGU10 CDRL1v2 |
| SEQ ID NO: 75 | CACGGCCACACGTCGAAGGCT | MGU10 CDRL1v3 |
| SEQ ID NO: 76 | GTCAATTCCGACGGATCTCAT | MGU10 CDRL2v1 |
| SEQ ID NO: 77 | GTCAACTCCGACGGCAGTCAT | MGU10 CDRL2v2 |
| SEQ ID NO: 78 | GTGAACAGCGACGGCAGCCAC | MGU10 CDRL2v3 |
| SEQ ID NO: 79 | CAGGCCTGGGACAGCGGCATCTGGGTG | MGU10 CDRL3v1 |
| SEQ ID NO: 80 | CAGGCCTGGGACTCAGGCATATGGGTA | MGU10 CDRL3v2 |
| SEQ ID NO: 81 | CAGGCCTGGGACTCAGGTATTTGGGTA | MGU10 CDRL3v3 |
| SEQ ID NO: 82 | GGCTTCAGCTTCAGCAGCTACGCC | MGH2 CDRH1v1 |
| SEQ ID NO: 83 | GGATTCTCATTCTCTTCCTACGCC | MGH2 CDRH1v2 |
| SEQ ID NO: 84 | GGGTTTAGCTTCTCTTCCTATGCG | MGH2 CDRH1v3 |
| SEQ ID NO: 85 | ACTCGGTATGACGGTTCGAATAAG | MGH2 CDRH2v1 |
| SEQ ID NO: 86 | ACAAGGTATGATGGTTCAAACAAG | MGH2 CDRH2v2 |
| SEQ ID NO: 87 | ACCAGATACGACGGCAGCAACAAG | MGH2 CDRH2v3 |
| SEQ ID NO: 88 | GCCAAGGTGGGCGACGGCACCGTGGCCGG | MGH2 CDRH3v1 |
| | CACCATCGACTAC | |
| SEQ ID NO: 89 | GCTAAGGTTGGGGACGGCACCGTTGCCGGC | MGH2 CDRH3v2 |
| | ACAATCGACTAT | |
| SEQ ID NO: 90 | GCTAAGGTAGGCGATGGTACGGTAGCAGGC | MGH2 CDRH3v3 |
| | ACGATAGATTAC | |
| SEQ ID NO: 91 | CAGAGCCTGGTGTACAGCGACGGCAACACC | MGH2 CDRL1v1 |
| | TAC | |
| SEQ ID NO: 92 | CAATCTCTCGTGTACTCCGACGGCAATACCTA | MGH2 CDRL1v2 |
| | C | |
| SEQ ID NO: 93 | CAGTCCCTCGTGTACTCAGATGGAAATACGT | MGH2 CDRL1v3 |
| | AT | |
| SEQ ID NO: 94 | AAAGTTAGT | MGH2 CDRL2v1 |
| SEQ ID NO: 95 | AAGGTCAGC | MGH2 CDRL2v2 |
| SEQ ID NO: 96 | AAGGTGAGC | MGH2 CDRL2v3 |
| SEQ ID NO: 97 | ATGCAGGGCACCCACTGGTGGACC | MGH2 CDRL3v1 |
| SEQ ID NO: 98 | ATGCAGGGTACCCATTGGTGGACC | MGH2 CDRL3v2 |
| SEQ ID NO: 99 | ATGCAGGGTACCCACTGGTGGACT | MGH2 CDRL3v3 |

Amino acid sequences

| | | |
|---|---|---|
| SEQ ID NO: 100 | QVQLVESGGGVVQPGRSLRLSCAASGFAFSNY | MGU10v2_LS heavy |
| | GMNWVRQAPGKGLEWVAVIWHDGSLKYYTQ | chain |
| | SVKGRFTISRDNAKNTLFLQMDSLSADDTAMYY | |
| | CTVWYLETPDEGFDIWGRGTMVTVSSASTKGPS | |
| | VFPLAPSSKSTSGGTAALGCLVKDYFPEPVTVSW | |
| | NSGALTSGVHTFPAVLQSSGLYSLSSVVTVPSSSL | |
| | GTQTYTCNVNHKPSNTKVDKRVEPKSCDKTHTC | |
| | PPCPAPELLGGPSVFLFPPKPKDTLMISRTPEVTCV | |
| | VVDVSHEDPEVKFNWYVDGVEVHNAKTKPREE | |
| | QYNSTYRVVSVLTVLHQDWLNGKEYKCKVSNK | |
| | ALPAPIEKTISKAKGQPREPQVYTLPPSREEMTKN | |
| | QVSLTCLVKGFYPSDIAVEWESNGQPENNYKTT | |
| | PPVLDSDGSFFLYSKLTVDKSRWQQGNVFSCSV | |
| | LHEALHSHYTQKSLSLSPGK | |
| SEQ ID NO: 101 | QVQLVESGGGVVQPGRSLRLSCAASGFAFSNY | MGU10_LS heavy |
| | GMNWVRQAPGKGLEWVAVTWHDGSLKYYTQ | chain |
| | SVKGRFTISRDNAKNTLFLQMDSLSADDTAMYY | |
| | CTVWYLETPDDGFDIWGRGTMVTVSSASTKGP | |
| | SVFPLAPSSKSTSGGTAALGCLVKDYFPEPVTVS | |
| | WNSGALTSGVHTFPAVLQSSGLYSLSSVVTVPSS | |
| | SLGTQTYICNVNHKPSNTKVDKRVEPKSCDKTH | |
| | TCPPCPAPELLGGPSVFLFPPKPKDTLMISRTPEVT | |
| | CVVVDVSHEDPEVKFNWYVDGVEVHNAKTKPR | |
| | EEQYNSTYRVVSVLTVLHQDWLNGKEYKCKVS | |
| | NKALPAPTEKTISKAKGQPREPQVYTLPPSREEMT | |
| | KNQVSLTCLVKGFYPSDIAVEWESNGQPENNYK | |
| | TTPPVLDSDGSFFLYSKLTVDKSRWQQGNVFSC | |
| | SVLHEALHSHYTQKSLSLSPGK | |
| SEQ ID NO: 102 | QVQLVESGGGVVQPGGSLRLSCTASGFSFSSYA | MGH2/MGH2v1_LS |
| | MHWVRQAPGKGLEWVAYTRYDGSNKFYLDSV | heavy chain |
| | QGRFTISRDNSKNTLYLEMDSLRLEDTAVYFCAK | |
| | VGDGTVAGTIDYWGQGTLVTVSSASTKGPSVFP | |
| | LAPSSKSTSGGTAALGCLVKDYFPEPVTVSWNSG | |
| | ALTSGVHTFPAVLQSSGLYSLSSVVTVPSSSLGTQ | |
| | TYICNVNHKPSNTKVDKRVEPKSCDKTHTCPPC | |
| | PAPELLGGPSVFLFPPKPKDTLMISRTPEVTCVVV | |
| | DVSHEDPEVKFNWYVDGVEVHNAKTKPREEQY | |
| | NSTYRVVSVLTVLHQDWLNGKEYKCKVSNKALP | |
| | APIEKTISKAKGQPREPQVYTLPPSREEMTKNQVS | |
| | LTCLVKGFYPSDIAVEWESNGQPENNYKTTPPVL | |
| | DSDGSFFLYSKLTVDKSRWQQGNVFSCSVLHEA | |
| | LHSHYTQKSLSLSPGK | |
| SEQ ID NO: 103 | ASTKGPSVFPLAPSSKSTSGGTAALGCLVKDYFPE | heavy chain constant |
| | PVTVSWNSGALTSGVHTFPAVLQSSGLYSLSSVV | region LS |
| | TVPSSSLGTQTYICNVNHKPSNTKVDKRVEPKSC | |
| | DKTHTCPPCPAPELLGGPSVFLFPPKPKDTLMISR | |
| | TPEVTCVVVDVSHEDPEVKFNWYVDGVEVHNA | |
| | KTKPREEQYNSTYRVVSVLTVLHQDWLNGKEYK | |
| | CKVSNKALPAPIEKTTSKAKGQPREPQVYTLPPSRE | |
| | EMTKNQVSLTCLVKGFYPSDIAVEWESNGQPEN | |
| | NYKTTPPVLDSDGSFFLYSKLTVDKSRWQQGNV | |
| | FSCSVLHEALHSHYTQKSLSLSPGK | |
| SEQ ID NO: 104 | QVQLVESGGGVVQPGRSLRLSCAASGFAFSNY | MGU10v8 VH |
| | GMNWVRQAPGKGLEWVAVIWHDGSLKYYTQ | |
| | SVKGRFTISRDNAKNTLFLQMDSLSAEDTAMYY | |
| | CTVWYLETPDEGFDIWGRGTMVTVSS | |
| SEQ ID NO: 105 | KQPADGNPDPNANPNVDPN | NPDP19-peptide |

Nucleic acid sequences

| | | |
|---|---|---|
| SEQ ID NO: 106 | ACCGGTGTACATTCTCAGGTGCAGCTGGTGG | MGU10v8 VH |
| | AGTCCGGAGGAGGAGTGGTGCAGCCAGGC | |
| | AGGAGCCTGAGGCTGTCTTGCGCTGCTTCCG | |
| | GATTCGCCTTTAGCAACTACGGCATGAATTG | |
| | GGTGAGGCAGGCTCCTGGCAAGGGACTGG | |
| | AGTGGGTGGCTGTGATCTGGCACGACGGCA | |
| | GCCTGAAGTACTATACACAGTCTGTGAAGGG | |
| | CAGATTCACCATCTCTCGCGATAACGCTAAG | |
| | AATACACTGTTTCTGCAGATGGACTCTCTGTC | |
| | CGCCGAGGATACCGCTATGTACTATTGTACA | |
| | GTGTGGTATCTGGAGACCCCAGACGAGGGC | |
| | TTCGATATCTGGGGCAGAGGCACCATGGTG | |
| | ACAGTGTCCAGCGCGTCGAC | |

1. An antibody, or an antigen-binding fragment thereof, comprising (i) the heavy chain CDR1, CDR2, and CDR3 sequences as set forth in SEQ ID NO: 1, SEQ ID NO: 2, and SEQ ID NO: 12, respectively, and the light chain CDR1, CDR2, and CDR3 sequences as set forth in SEQ ID NO: 4, SEQ ID NO: 5, and SEQ ID NO: 6, respectively; or (ii) the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in SEQ ID NO: 4, SEQ ID NO: 5, and SEQ ID NO: 14, respectively; or (iii) the heavy chain CDR1, CDR2, and CDR3 sequences as set forth in SEQ ID NO: 1, SEQ ID NO: 2, and SEQ ID NO: 12, respectively, and the light chain CDR1, CDR2, and CDR3 sequences as set forth in SEQ ID NO: 4, SEQ ID NO: 5, and SEQ ID NO: 14, respectively; or (iv) the heavy chain CDR1, CDR2, and CDR3 sequences as set forth in SEQ ID NO: 17, SEQ ID NO: 18, and SEQ ID NO: 19, respectively, and the light chain CDR1, CDR2, and CDR3 sequences as set forth in SEQ ID NO: 20, SEQ ID NO: 21, and SEQ ID NO: 28, respectively; or (v) the heavy chain CDR1, CDR2, and CDR3 sequences as set forth in SEQ ID NO: 17, SEQ ID NO: 18, and SEQ ID NO: 19, respectively, and the light chain CDR1, CDR2, and CDR3 sequences as set forth in SEQ ID NO: 20, SEQ ID NO: 22, and SEQ ID NO: 28, respectively wherein the antibody, or antigen-binding fragment thereof binds to Plasmodium falciparum sporozoites.

2. The antibody, or an antigen-binding fragment thereof, of claim 1, wherein the antibody, or the antigen-binding fragment thereof, comprises:
(i) a heavy chain variable region comprising an amino acid sequence as set forth in SEQ ID NO: 13 or an amino acid sequence having at least 70% identity to SEQ ID NO: 13 and a light chain variable region comprising an amino acid sequence as set forth in SEQ ID NO: 8 or an amino acid sequence having at least 70% identity to SEQ ID NO: 8; or
(ii) a heavy chain variable region comprising an amino acid sequence as set forth in SEQ ID NO: 7 or an amino acid sequence having at least 70% identity to SEQ ID NO: 7 and a light chain variable region comprising an amino acid sequence as set forth in SEQ ID NO: 15 or an amino acid sequence having at least 70% identity to SEQ ID NO: 15; or
(iii) a heavy chain variable region comprising an amino acid sequence as set forth in SEQ ID NO: 16 or an amino acid sequence having at least 70% identity to SEQ ID NO: 16 and a light chain variable region comprising the amino acid sequence as set forth in SEQ ID NO: 8 or the amino acid sequence having at least 70% identity to SEQ ID NO: 8; or
(iv) a heavy chain variable region comprising an amino acid sequence as set forth in SEQ ID NO: 11 or an amino acid sequence having at least 70% identity to SEQ ID NO: 11 and a light chain variable region comprising the amino acid sequence as set forth in SEQ ID NO: 15 or the amino acid sequence having at least 70% identity to SEQ ID NO: 15; or
(v) a heavy chain variable region comprising the amino acid sequence as set forth in SEQ ID NO: 13 or the amino acid sequence having at least 70% identity to SEQ ID NO: 13 and a light chain variable region comprising the amino acid sequence as set forth in SEQ ID NO: 15 or the amino acid sequence having at least 70% identity to SEQ ID NO: 15; or
(vi) a heavy chain variable region comprising the amino acid sequence as set forth in SEQ ID NO: 16 or the amino acid sequence having at least 70% identity to SEQ ID NO: 16 and a light chain variable region comprising the amino acid sequence as set forth in SEQ ID NO: 15 or the amino acid sequence having at least 70% identity to SEQ ID NO: 15; or
(vii) a heavy chain variable region comprising the amino acid sequence as set forth in SEQ ID NO: 24 or the amino acid sequence having at least 70% identity to SEQ ID NO: 24 and a light chain variable region comprising the amino acid sequence as set forth in SEQ ID NO: 29 or the amino acid sequence having at least 70% identity to SEQ ID NO: 29; or
(viii) a heavy chain variable region comprising an amino acid sequence as set forth in SEQ ID NO: 104 or an amino acid sequence having at least 70% identity to SEQ ID NO: 104 and a light chain variable region comprising the amino acid sequence as set forth in SEQ ID NO: 8 or the amino acid sequence having at least 70% identity to SEQ ID NO: 8; or
(ix) a heavy chain variable region comprising the amino acid sequence as set forth in SEQ ID NO: 104 or the amino acid sequence having at least 70% identity to SEQ ID NO: 104 and a light chain variable region comprising the amino acid sequence as set forth in SEQ ID NO: 15 or the amino acid sequence having at least 70% identity to SEQ ID NO: 15.

3-8. (canceled)

9. The antibody, or the antigen-binding fragment thereof, of claim 1, wherein the antibody, or the antigen-binding fragment thereof, comprises the heavy chain CDR1, CDR2, and CDR3 sequences as set forth in SEQ ID NO: 1, SEQ ID NO: 2, and SEQ ID NO: 12, respectively, and the light chain CDR1, CDR2, and CDR3 sequences as set forth in SEQ ID NO: 4, SEQ ID NO: 5, and SEQ ID NO: 6, respectively.

10. The antibody, or the antigen-binding fragment thereof, of claim 9, wherein the antibody, or the antigen-binding fragment thereof, comprises (A) (i) a heavy chain variable region comprising an amino acid sequence according to SEQ ID NO: 13 or an amino acid sequence having at least 70% identity to SEQ ID NO: 13 and (ii) a light chain variable region comprising an amino acid sequence according to SEQ ID NO: 8 or an amino acid sequence having at least 70% identity to SEQ ID NO: 8, (B) (i) a heavy chain variable region comprising an amino acid sequence according to SEQ ID NO: 16 or an amino acid sequence having at least 70% identity to SEQ ID NO: 16 and (ii) a light chain variable region comprising an amino acid sequence according to SEQ ID NO: 8 or an amino acid sequence having at least 70% identity to SEQ ID NO: 8, or (C) (i) a heavy chain variable region comprising an amino acid sequence according to SEQ ID NO: 104 or an amino acid sequence having at least 70% identity to SEQ ID NO: 104 and (ii) a light chain variable region comprising an amino acid sequence according to SEQ ID NO: 8 or an amino acid sequence having at least 70%-identity to SEQ ID NO: 8.

11-12. (canceled)

13. The antibody, or the antigen-binding fragment thereof, of claim 1, wherein the antibody, or the antigen-binding fragment thereof, comprises the heavy chain CDR1, CDR2, and CDR3 sequences as set forth in SEQ ID NO: 1, SEQ ID NO: 2, and SEQ ID NO: 3, respectively, and the light chain CDR1, CDR2, and CDR3 sequences as set forth in SEQ ID NO: 4, SEQ ID NO: 5, and SEQ ID NO: 14, respectively.

14. The antibody, or the antigen-binding fragment thereof, of claim 13, wherein the antibody, or the antigen-binding fragment thereof, comprises (A) (i) a heavy chain variable region comprising an amino acid sequence according to SEQ ID NO: 7 or an amino acid sequence having at least 70% identity to SEQ ID NO: 7 and (ii) a light chain variable region comprising an amino acid sequence according to SEQ ID NO: 15 or an amino acid sequence having at least 70% identity to SEQ ID NO: 15, or (B) (i) a heavy chain variable region comprising an amino acid sequence according to SEQ ID NO: 11 or an amino acid sequence having at least 70% identity to SEQ ID NO: 11 and (ii) a light chain variable region comprising an amino acid sequence according to SEQ ID NO: 15 or an amino acid sequence having at least 70% identity to SEQ ID NO: 15.

15. (canceled)

16. The antibody, or the antigen-binding fragment thereof, of claim 1, wherein the antibody, or the antigen-binding fragment thereof, comprises the heavy chain CDR1, CDR2, and CDR3 sequences as set forth in SEQ ID NO: 1, SEQ ID NO: 2, and SEQ ID NO: 12, respectively, and the light chain CDR1, CDR2, and CDR3 sequences as set forth in SEQ ID NO: 4, SEQ ID NO: 5, and SEQ ID NO: 14, respectively.

17. The antibody, or the antigen-binding fragment thereof, of claim 16, wherein the antibody, or the antigen-binding fragment thereof, comprises (A) (i) a heavy chain variable region comprising an amino acid sequence according to SEQ ID NO: 13 or an amino acid sequence having at least 70% identity to SEQ ID NO: 13 and (ii) a light chain variable region comprising an amino acid sequence according to SEQ ID NO: 15 or an amino acid sequence having at least 70% identity to SEQ ID NO: 15, (B) (i) a heavy chain variable region comprising an amino acid sequence according to SEQ ID NO: 16 or an amino acid sequence having at least 70% identity to SEQ ID NO: 16 and (ii) a light chain variable region comprising an amino acid sequence according to SEQ ID NO: 15 or an amino acid sequence having at least 70% identity to SEQ ID NO: 15, or (C) (i) a heavy chain variable region comprising an amino acid sequence according to SEQ ID NO: 104 or an amino acid sequence having at least 70% identity to SEQ ID NO: 104 and (ii) a light chain variable region comprising an amino acid sequence according to SEQ ID NO: 15 or an amino acid sequence having at least 70% identity to SEQ ID NO: 15.

18-19. (canceled)

20. The antibody, or the antigen-binding fragment thereof, of claim 1, wherein the antibody, or the antigen-binding fragment thereof, comprises the heavy chain CDR1, CDR2, and CDR3 sequences (i) as set forth in SEQ ID NO: 17, SEQ ID NO: 18, and SEQ ID NO: 19, respectively, and the light chain CDR1, CDR2, and CDR3 sequences as set forth in SEQ ID NO: 20, SEQ ID NO: 21, and SEQ ID NO: 28, respectively or (ii) as set forth in SEQ ID NO: 17, SEQ ID NO: 18, and SEQ ID NO: 19, respectively, and the light chain CDR1, CDR2, and CDR3 sequences as set forth in SEQ ID NO: 20, SEQ ID NO: 22, and SEQ ID NO: 28, respectively.

21. (canceled)

22. The antibody, or the antigen-binding fragment thereof, of claim 20, wherein the antibody, or the antigen-binding fragment thereof, comprises (i) a heavy chain variable region comprising an amino acid sequence according to SEQ ID NO: 24 or an amino acid sequence having at least 70% identity to SEQ ID NO: 24 and (ii) a light chain variable region comprising an amino acid sequence according to SEQ ID NO: 29 or an amino acid sequence having at least 70% identity to SEQ ID NO: 29.

23. An antibody, or an antigen-binding fragment thereof, comprising (i) a heavy chain variable region comprising an amino acid sequence as set forth in SEQ ID NO: 11 and a light chain variable region comprising the amino acid sequence as set forth in SEQ ID NO: 8; or (ii) a heavy chain variable region comprising an amino acid sequence as set forth in SEQ ID NO: 24 and a light chain variable region comprising the amino acid sequence as set forth in SEQ ID NO: 29.

24-36. (canceled)

37. The antibody, or an antigen-binding fragment thereof, of claim 1, wherein the antibody, or the antigen-binding fragment thereof, is a human antibody and/or a monoclonal antibody.

38. (canceled)

39. The antibody of claim 1, wherein the antibody comprises an Fc moiety.

40. The antibody, or an antigen-binding fragment thereof, of claim 39, wherein the antibody, or the antigen-binding fragment thereof, comprises the mutations M428L and/or N434S in the heavy chain constant region.

41. The antibody of claim 1, wherein the antibody is of the IgG type.

42. (canceled)

43. The antibody, or an antigen-binding fragment thereof, of claim 1, wherein the antibody, or the antigen-binding fragment thereof, is purified, a single-chain antibody, a Fab, a Fab′, a F(ab′)2, a Fv, or a scFv.

44-45. (canceled)

46. The antibody, or an antigen-binding fragment thereof, of claim 1, wherein the variable region of the heavy chain of the antibody, or of the antigen-binding fragment thereof, is encoded by a nucleic acid comprising a gene of the VH3 gene family.

47-48. (canceled)

49. A composition comprising (i) a nucleic acid molecule comprising a polynucleotide encoding the antibody, or an antigen-binding fragment thereof, of claim 1 or (ii) a first nucleic acid molecule comprising a polynucleotide encoding the heavy chain of the antibody, or antigen-binding fragment thereof, of claim 1, and a second nucleic acid molecule comprising a polynucleotide encoding the light chain of the same antibody, or antigen-binding fragment thereof.

50-64. (canceled)

65. A vector or combination of vectors comprising the nucleic acid molecule or molecules of claim 49.

66. (canceled)

67. A cell expressing the antibody, or an antigen-binding fragment thereof, of claim 1, or comprising the vector or combination of vectors of claim 65.

68. A pharmaceutical composition comprising the antibody, or an antigen-binding fragment thereof, of claim 1, the nucleic acid or combination of nucleic acids of claim 49, or the vector or combination of vectors of claim 65.

69-70. (canceled)

71. A method of reducing malaria, or lowering the risk of Plasmodium falciparum infection, comprising: administering to a subject in need thereof, a therapeutically effective amount of the antibody, or an antigen-binding fragment thereof, of claim 1, the nucleic acid or combination of nucleic acids of claim 49, the vector or combination of vectors of claim 65, or the pharmaceutical composition of claim 68.
